(19) United States
(12) Patent Application Publication
(10) Pub. No.: US 2013/0237427 A1
(43) Pub. Date: Sep. 12, 2013

(54) Y-SHAPED PROBE AND VARIANT THEREOF, AND DNA MICROARRAY, KIT AND GENETIC ANALYSIS METHOD USING THE SAME

(71) Applicants: Woo Chul Moon, Seoul (KR); Myung Ryurl Oh, Gyeonggi-do (KR)

(72) Inventors: Woo Chul Moon, Seoul (KR); Myung Ryurl Oh, Gyeonggi-do (KR)

(73) Assignee: GOODGENE INC., SEOUL (KR)

(21) Appl. No.: 13/581,371

(22) PCT Filed: Mar. 26, 2010
(86) PCT No.: PCT/KR2010/001878

§ 371 Date: Oct. 2, 2012

(30) Foreign Application Priority Data
Feb. 26, 2010 (KR) 10-2010-0018008

(51) Int. Cl. C12Q 1/68

(52) U.S. Cl. CPC C12Q 1/689; C12Q 1/6888; C12Q 1/6886
USPC 506/2; 536/24.3; 506/16; 506/9

(57) ABSTRACT

The present disclosure relates to a Y-shaped nucleotide probe having two probe parts in one body, which provides improved sensitivity, specificity and accuracy in genotype and genetic analysis and thus is widely applicable to diagnosis and a variant thereof (d- or b-shaped probe), and a DNA microarray, a kit and a genetic analysis method using the same. The Y-shaped probe comprises a left-side probe part, a left-side stem part, a linker part, a right-side stem part and a right-side probe part. The DNA microarray of the present disclosure can improve accuracy of test by testing the same gene simultaneously or by testing two different target genes at once. Especially, since both the target gene and the control gene can be tested simultaneously from one spot, error can be reduced, quantitative analysis is possible and standardization is easy. The Y-shaped probe of the present disclosure may be widely used for genotyping, analysis of gene expression, analysis of mutation or SNP, diagnosis and prediction of diseases, determination of therapeutic regimen, or the like.

### TECHNICAL FIELD

The present disclosure relates to a Y-shaped nucleotide probe having two probe parts in one body, which provides improved sensitivity, specificity and accuracy in genotype and genetic analysis and thus is widely applicable to diagnosis and a variant thereof (d- or b-shaped probe), and a DNA microarray, a kit and a genetic analysis method using the same.

### BACKGROUND ART

A DNA microarray or a DNA chip refers to tens to billions of gene probes spotted on a solid support such as a glass slide. After placing nucleic acids such as DNA, RNA, cDNA, cRNA, microRNA, polymerase chain reaction (PCR) product, etc., which have been obtained from tissue, cell or body fluid sample and are labeled with e.g. a fluorescent dye, on the DNA microarray, hybridization or sequencing reaction can be performed and signals from the label as a result of the reaction may be analyzed using, for example, a fluorescence scanner. The DNA microarray allows analysis of change in gene expression or genotype of large numbers of genes through a single experiment. The DNA microarray has become an essential tool in genetic researches or clinical practices, and is widely used in basic researches including the study of the function of genes and genomes as well as in clinical practices for understanding the mechanism of genetic diseases, establishing diagnosis protocols, elucidating the action mechanisms and side effects of particular drugs, determining therapeutic strategies, or the like (Petrik J. Diagnostic applications of microarrays. Transfusion Medicine. 2006; 16: 233-247; Wheelan S J, Murillo F M and Boeke J D. The incredible shrinking world of DNA microarrays. Mol Biosyst. 2008; 4(7): 726-732; Li X, Quigg R J, Zhou J, Gu W, Nagesh Rao P, Reed E F. Clinical utility of microarrays: current status, existing challenges and future outlook. Current Genomics. 2008; 9(7): 466-74).

There are two types of DNA microarrays, depending on the probes spotted on the microarray: i.e., the oligonucleotide microarray and other microarrays on which cDNA or PCR product is spotted. Most of the currently commercially available microarrays are oligonucleotide microarrays. The oligonucleotide microarray can be classified into two types depending on how they are fabricated. The first type is one in which oligonucleotides are synthesized directly on the solid support. Examples include the Affymetrix's photolithography chip, the Agilent's ink-jet chip, the Combimatrix's electronically synthesized chip, the Nimblegen's photosynthesized chip, and so forth. The other type is one in which previously and separately prepared oligonucleotide probes are spotted on the solid support. This later type is more widely used and representative examples include the Applied Biosystems, Inc. (ABI)'s product, the CodeLink's product and the Illumina's product. Linear, single-stranded oligonucleotide probes of 18-75 base pairs (bp) are spotted on these microarray products, and the number of the spots varies from 12,000 to 1,072,000,000 (Wheelan S J, Murillo F M and Boeke J D. The incredible shrinking world of DNA microarrays. Mol Biosyst. 2008; 4(7): 726-732).

The DNA microarray performs the three tasks that have been performed by genetic testing. It is distinguished from the previous genetic testing technique in that it allows high-throughput analysis of multiple genes at once, thereby greatly reducing cost and time and being applicable to clinical diagnosis.

The first testing using the DNA microarray is qualitative analysis of investigating whether a gene having a specific nucleotide sequence is present in the sample. For example, a microarray is prepared using specific nucleotide sequences of a bacterial gene causing a disease as probes and hybridization is performed after placing a nucleic acid sample on the microarray to detect the causative target gene in a “looking for a needle in a haystack” manner. Through this genotyping, human papilloma virus (HPV) causing cervical cancer, influenza virus causing flu, and microorganisms causing sexually transmitted infections can be accurately identified, not only the species but also the strains and subspecies. Furthermore, diagnosis of cancer is also possible by detecting the presence of oncogenes. Also, the malignancy or prognosis of the bacteria or cancer to be detected as well as drug reactions and side effects can be predicted. This can be achieved with a low-density microarray, which can be fabricated easily at low cost, is useful for clinical practice and can be easily commercialized (Yoo S M, Choi J H, Lee S Y, Yoo N C. Applications of DNA microarray in disease diagnostics. J Microbiol Biotechnol. 2009; 19(7): 635-46).

The second testing using the DNA microarray is quantitative analysis of identifying the quantity of a gene having a specific nucleotide sequence present in the sample, as was achieved with the first cDNA microarray (Shena M, Shalon D, Davis R W, Broiwn P O. Quantitative monitoring of gene expression pattern with a complementary DNA microarray. Science. 1995; 270: 467-470). After spotting a plurality of probes for the gene to be detected on the microarray, the RNA, cDNA or cRNA of the target substance and the control substance labeled with different fluorescent dyes is placed on the microarray and hybridization is performed. Then, the difference in gene expression between the two groups is compared.

The third testing using the DNA microarray is identification of the change in nucleotide sequences of a gene. Specifically, single nucleotide polymorphism (SNP), point mutation or deletion is examined. In addition, it is also possible to identify the copy number of a specific gene. Usually, a DNA chip is fabricated by spotting oligonucleotide probes differing by a single nucleotide, for example, as a wild type and a mutant (or variant) type. Then, after placing a sample DNA, cDNA or PCR product, hybridization is performed under highly stringent conditions to identify a perfectly matching probe. That is to say, allele-specific oligonucleotide hybridization (ASH) or sequencing by hybridization (SBH) is carried out on the microarray. A number of companies are providing microarrays allowing allele-specific oligonucleotide hybridization of SNPs in the human genome in whole or in part. For example, the Affymetrix's SNP chip uses various perfect match-type and mismatch-type oligonucleotide probes for one SNP (Rabbee N and Speed T P. A genotype calling algorithm for Affymetrix SNP arrays. Bioinformatics 2006; 22: 7-12; Liu W M, X. Yang X D G, Matsuzaki H, Huang J, Mei R, Ryder T B, Webster T A, Dong S, Liu G, K. W. Jones K W, G. C. Kennedy G C and Kulp D. Algorithms for large-scale genotyping microarrays, Bioinformatics. 2003; 19: 2397-2403).

However, there are many difficulties in actually detecting the difference in a single nucleotide for multiple targets using the DNA microarray. Accordingly, potent rival products of the DNA microarray are being developed. Examples include Illumina's Solexa, Helicos's instrument, Roche's 454 and Applied Biosystems's SOLiD, which are high-throughput nucleotide sequencing instruments allowing reading of one gigabase of a DNA sequence at once. They excel the DNA microarray in the quantity of the nucleotide sequences that can be analyzed and are capable of reading the entire nucleotide sequence of human genome in a few days (Wheelan S J, Murillo F M and Boeke J D. The incredible shrinking world of DNA microarrays. Mol Biosyst. 2008; 4(7): 726-732).

Despite the long history since the first report on the microarray by Shenna in 1995, only few products are used for clinical practices. In the US, the AmpliChip CYP450 for pharmacogenetic testing, the MammaPrint chip for diagnosis of breast cancer, the AmpliChip p53 test for analysis of p53 mutation, the Pathwork Tissue of Origin test for examining the cause of cancer, and the BAC array test for testing chromosome abnormality are used under approval of the USFDA (Li X, Quigg R J, Zhou J, Gu W, Nagesh Rao P, Reed E F. Clinical utility of microarrays: current status, existing challenges and future outlook. Curr Genomics. 2008; 9(7): 466-74; Heller T, Kirchheiner J, Armstrong V W, Luthe H, Tzvetkov M, Brockmoller J, Oellerich M. AmpliChip CYP450 GeneChip: a new gene chip that allows rapid and accurate CYP2D6 genotyping. Ther. Drug Monit. 2006; 28: 673-677; Mook S, Van't Veer L J, Rutgers E J, Piccart-Gebhart M J, Cardoso F. Individualization of therapy using Mammaprint: from development to the MINDACT Trial. Cancer Genomics Proteomics. 2007; 4: 147-155; Lawrence H J, Truong S, Patten N, Nakao A, Wu L. Detection of p53 mutations in cancer by the amplichip p53 test). And, in Korea, Europe and other places, a chip for diagnosing the genotype of human papilloma virus (HPV) is marked under approval.

For the DNA microarray to be widely used for clinical diagnosis, many problems need to be solved. All types of DNA microarrays commonly have the problem of background noise or the nonspecific signal, which causes difficulties in signal analysis or product standardization. This problem incurs controversy over the accuracy or value of the DNA microarray (Allison D B, Cui X Q, Page G P and Sabripou M. Microarray data analysis: From disarray to consolidation and consensus, Genetics. 2006; 7: 55-65; Draghici S P, Eklund S P K and Eklund and Szallasi Z. Reliability and reproducibility issues in DNA microarray measurements. Trends in Genetics. 2006; 22: pp. 101-109; Kothapalli R, Yoder S J, Mane S and Loughran T P, Microarray results: How accurate are they?. BMC Bioinformatics. 2002; 3: 22).

Although numerous tests can be performed at once on the spots of the DNA microarray, it is not easy to accurately analyze and interpret the data statistically. In general, an error smaller than 5% is regarded as acceptable (i.e., p=0.05) in statistical analysis. However, if the number of the spots on the microarray is from tens of millions to billions and about 5% of them, i.e. millions to tens of millions, are false positive or false negative, this would be a severe, huge error. Several microarrays may be used to avoid this problem, but it costs a lot since the individual microarrays are expensive. In actual experiments using the microarray, it is not infrequent that the result varies for each experiment and for different individual microarrays. Different results are found sometimes even in the spots on the same microarray. One of the biggest reasons of this problem is because the control group for experiment is not established. In other words, the problem arises because the internal reference is not set distinctly for each spot during the hybridization experiment on the DNA microarray.

Two types of errors may occur during the DNA microarray experiment, which are one intrinsic to the sample or purpose and one attributable to the microarray itself or test procedure. The former is related with the heterogeneity and diversity of the sample, changes depending on physiological conditions, interactions between the gene and the environment, and so forth. The error occurring from the DNA microarray itself is called the slide effect and is related with, for example, the kind of the solid support, i.e. the glass slide, of the DNA microarray and its surface chemistry, the pin used to spot the probes, the amount of the probes for each spot, the interaction between the probe and the glass slide, how well the probes are fixed on the glass slide, and so forth. Further, it is important how well the hybridization occurs, which depends on temperature, time and buffer solution conditions. It is also important how well the sample nucleic acid is labeled with the label (Bakay M, Chen Y W, Borup R, Zhao P, Nagaraju K and E. P. Hoffman E P. Sources of variability and effect of experimental approach on expression profiling data interpretation. BMC Bioinformatics. 2002; 3: 4; Han E S, Wu Y, McCarter R, Nelson J F, Richardson A and Hilsenbeck S S. Reproducibility, sources of variability, pooling, and sample size: Important considerations for the design of high-density oligonucleotide array experiments. Journal of Gastroenterology. 2004; 59: 306-315; Huber W, Heydebreck A, Sultmann H, Poustka A and Vingron M, Variance stabilization applied to microarray data calibration and to the quantification of differential expression, Bioinformatics. 2002; 18: 96-104; Molloy M P, Brzezinski E E, Hang J Q, McDowell M T and VanBogelen R A. Overcoming technical variation and biological variation in quantitative proteomics. Proteomics. 2003; 3: 1912-1919; Oleksiak M F, G. A. Churchill G A and D. L. Crawford, Variation in gene expression within and among natural populations, Nature Genetics. 2002; 32: 261-266; Spruill S E, Hardy J L S and Weir B. Assessing sources of variability in microarray gene expression data. BioTechniques. 2002: 916-923; Whitney A R, Diehn M, Popper S J, Alizadeh A A, J. C. Boldrick J C, Relman D A and Brown P O. Individuality and variation in gene expression patterns in human blood, Proc. Natl. Acad. Sci. USA. 2003; 100: 1896-1901; Zakharkin S O, Kim K, Mehta T, Chen L, Barnes S, Scheirer K E, Parrish R S, Allison D B and Page G P. Sources of variation in Affymetrix microarray experiments. BMC Bioinformatics. 2005; 6: 214).

Another problem that may occur during the DNA microarray experiment is related with the probes. As described above, linear, single-stranded oligonucleotide probes are used in most of the currently employed DNA microarrays. However, these probes have the problem that it is difficulty to establish an optimized condition unlike the hybridization occurring in liquid state, since the hybridization occurs on the solid support. Accordingly, it is critical to design and fabricate optimized oligonucleotide probes for a successful oligonucleotide microarray.

Therefore, designing of variously modified probes is attempted to improve the problem of the existing linear oligonucleotide probes. For example, there is an attempt to obtain nucleic acid analogs or mimics by modifying the bases, sugar rings or phosphodiester backbone of naturally occurring nucleic acids. Typical examples include peptide nucleic acid (PNA), locked nucleic acid (LNA), morpholino, and so forth. Since the melting temperature (Tₘ) of PNA or LNA is quite different from that of the common oligonucleotides, they are particularly useful in analyzing the single nucleotide polymorphism (SNP) or mutations (Karkare S, Bhatnagar D. Promising nucleic acid analogs and mimics: characteristic features and applications of PNA, LNA, and morpholino. Appl Microbiol Biotechnol. 2006; 71(5): 575-86; Tolstrup N, Nielsen P S, Kolberg J G, Frankel A M, Vissing H, Kauppinen S. OligoDesign: Optimal design of LNA (locked nucleic acid) oligonucleotide capture probes for gene expression profiling. Nucleic Acids Res. 2003; 31(13): 3758-62; Nhakeel S, Karim S and Ali A. Peptide nucleic acid (PNA)—a review. Journal of Chemical Technology and Biotechnology. 2006; 81: 892-899). However, they are not widely used for analysis of the expression of multiple genes and a microarray having an additional probe for an internal control gene as the Y-shaped probe of the present disclosure is not developed.

Another example of the probe modification is OligoSpawn. It is a technology of designing an overlapping oligonucleotide probe (overgo) from the massive UniGene database of expressed sequence tags (ESTs). OligoSpawn: a software tool for the design of overgo probes from large unigene datasets (Zheng J, Svensson J T, Madishetty K, Close T J, Jiang T, Lonardi S. OligoSpawn: a software tool for the design of overgo probes from large unigene datasets. BMC Bioinformatics. 2006 Jan. 9; 7: 7). Although this method is useful in designing the oligonucleotide probe quickly, a microarray having an additional probe for an internal control gene as the Y-shaped probe of the present disclosure is not developed as yet.

As another example of the probe modification, genomic DNA tiling arrays are actively developed and employed (Bertone P, Trifonov V, Rozowsky J S, Schubert F, Emanuelsson O, Karro J, Kao M Y, Snyder M, Gerstein M. Design optimization methods for genomic DNA tiling arrays. Genome Res. 2006; 16(2): 271-81; Wheelan S J, Murillo F M and Boeke J D. The incredible shrinking world of DNA microarrays. Mol Biosyst. 2008; 4(7): 726-732). This is a type of a multi-tiling oligonucleotide microarray allowing multiple genetic testing at once in which several subprobes are used for a single probe and multiple probes are spotted for each spot of the microarray. Multiple oligonucleotides at similar locations in a single probe are tiled and this technique is useful in identifying the genetic expression of the entire genome. However, the oligonucleotides are simply connected as a long linear chain, and it is impossible to identify the individual oligonucleotides and subprobes of each tiling probe. Accordingly, although the number of genes tested can be increased effectively, the sensitivity, specificity or reproducibility of test is not improved remarkably since an internal reference or control is included as in the Y-shaped probe of the present disclosure.

As described, for the DNA microarray to be widely applicable for clinical diagnosis, the oligonucleotide probe needs to be improved more and standardization of test method test method and result interpretation is important. Above all, there is a need to add a probe for an internal reference or control for each spot for the standardization. This is essential to solve the error occurring from the difference in hybridization between each spot of the microarray and the glass slide.

### DISCLOSURE

### Technical Problem

The present disclosure is directed to providing a novel Y-shaped probe comprising not only a target gene to be tested but also a control gene in each spot and a variant thereof, thereby improving the problem of the existing oligonucleotide microarray and allowing application to clinical diagnosis.

### Technical Solution

The inventors of the present disclosure have sought for a method of incorporating not only a probe for a target gene to be tested but also a probe for a control gene in a single spot in order to solve the above-described problems of the existing oligonucleotide microarray.

If the probe for the target gene and the probe for the control gene are incorporated as a single probe and the resulting probe is spotted in statistically sufficient numbers (e.g., 20 or more) on a microarray and if hybridization with a sample nucleic acid, e.g. DNA, RNA, cDNA, cRNA, micro RNA, etc. is performed after labeling the target gene and the control gene respectively with Cy-3 and Cy-5, the ratio of the signal of the target gene to that of the control gene (Cy3/Cy5) can be measured after excluding the background signal from each spot. Then, by calculating mean and standard deviation from the results from the multiple spots, more accurate statistical analysis will be possible. This allows experiments with a control group added for each spot, thus minimizing false positive and false negative errors, reducing errors occurring from different spots, and allowing easier normalization. Accordingly, the slide effect described above, i.e. the error occurring from the DNA microarray itself, can be minimized. Furthermore, since statistically significant data can be obtained through experiments using a small number of microarrays, the cost and time for each experiment can be decreased remarkably.

Thus, the inventors of the present disclosure have invented a Y-shaped probe having two oligonucleotide probe parts placed on a body in the form of the character Y and a method for spotting the probe on a solid support. In addition, they have designed a variant of the Y-shaped probe with one probe part being asymmetrically shorter, i.e. a d- or b-shaped probe.

Since the Y-shaped probe of the present disclosure has two oligonucleotide probes or peptide nucleic acid (PNA) probes, two hybridization reactions with nucleic acids having nucleotide sequences complementary to the probes may occur concurrently, and the reactions may be analyzed by using two different dyes. The inventors of the present disclosure have developed and fabricated the Y-shaped duplex oligonucleotide probe (hereinafter ‘Y-shaped probe’) as well as a method for testing genes using the same and a method for applying it for clinical diagnosis.

The probe of the present disclosure comprises five parts of a left-side probe part, a left-side stem part, a right-side stem part, a right-side probe part and a linker (or spacer). The left-side and right-side probe parts of the probe may comprise up to 150 oligonucleotides or PNAs and may comprise various nucleotide sequences depending on purposes. The nucleotide sequence of one of the two oligonucleotide probes is in a forward (sense; 5′→3′) direction and the other is in a reverse (antisense; 3′→5′) direction. The stem part comprises up to 40 complementary oligonucleotides and serves to support the probe parts. The stem part may have any nucleotide sequence. Specifically, it may have a telomere sequence. The linker serves to fix the probe parts and the stem part on a solid support such as a glass slide. An aldehyde-treated glass slide is commonly used as the support for a DNA microarray. In this case, the linker may be an internal amino modifier Cn dT (iAmMCnT). Also, a linker having a terminal biotin moiety may be used and it may be used to fix the probe parts and the stem part on a streptavidin-coated support.

A DNA microarray is completed by spotting the Y-shaped probe of the present disclosure on the support such as a glass slide using an arrayer. Then, a target nucleic acid to be tested, i.e. DNA, RNA, cDNA, cRNA, microRNA, etc., labeled with a fluorescent dye is placed thereon and a fluorescence signal generated as a result of hybridization may be analyzed using a fluorescence scanner. A one-, two- or four-color scanner may be selected depending on the purpose and method of test.

The Y-shaped oligonucleotide probe of the present disclosure has a number of advantages over the linear, single-stranded probe. First, since two probe parts are included in one probe, a more accurate analysis is possible through duplex testing. Second, since testing with the internal reference or control is performed concurrently, false negative result or false positive result can be minimized and, hence, the sensitivity and specificity of test can be improved. Third, more accurate statistical analysis is possible by avoiding errors occurring from different spots. Fourth, relative quantification is possible between the control substance and the target substance. Fifth, the stem part allows differentiation of melting temperature (Tₘ) or annealing temperature thermodynamically. Thus, it is expected that a clearer change will be detected when mutation of a single nucleotide is analyzed by allele-specific hybridization. Sixth, the stem part disposed between the probe parts and the linker and the glass slide support reduce steric hindrance or electromagnetic interference and allow the hybridization to occur more easily.

The present disclosure provides, first, a Y-shaped probe capable of analyzing the presence of a particular sequence of DNA or RNA specific for a disease or gene to be detected and a method for designing and fabricating the same, second, a biochip on which the Y-shaped probe is spotted and a method for fabricating the same, third, an effective method for PCR and fluorescence labeling of the biochip, fourth, a method for detecting a target gene and analyzing genotype, gene expression level and mutation of nucleotide sequence using the biochip, and, fifth, a method for using the same for clinical purposes.

Features of the Disclosure

In one general aspect, the present disclosure provides a Y-shaped nucleotide probe having a body and two probe parts.

Specifically, the probe of the present disclosure may comprise, in 5′→3′ direction, from left top to right top, (1) a left-side probe part, (2) a left-side stem part, (3) a linker part, (4) a right-side stem part and (5) a right-side probe part.

The present disclosure also provides a d-shaped nucleotide probe with (1) the left-side probe part of the Y-shaped probe removed and comprising (2) the left-side stem part, (3) the linker part, (4) the right-side stem part and (5) the right-side probe part.

The present disclosure also provides a b-shaped nucleotide probe with (5) the right-side probe part of the Y-shaped probe removed and comprising (1) the left-side probe part, (2) the left-side stem part, (3) the linker part and (4) the right-side stem part.

Specifically, the left-side stem part and the right-side stem part of the Y-, d- and b-shaped probes of the present disclosure may be oligonucleotides having complementary nucleotide sequences and bonded together, and each of the left-side stem part and the right-side stem part may comprise the nucleotide G in at least half of the entire nucleotide sequence.

Specifically, the left-side stem part and the right-side stem part may be oligonucleotides having complementary nucleotide sequences and bonded together, and the nucleotide sequence of the stem part may be a telomere nucleotide sequence.

Specifically, the left-side stem part or the right-side stem part may comprise at least one repeating nucleotide unit selected from TTGGG, TAGGG, TTGGGG, TTTGGG, TTAGGG, TTTGGGG, TTTAGGG, TTTTGGGG and TTTAGGGG.

Specifically, the left-side probe part or the right-side probe part may be an oligonucleotide having a nucleotide sequence complementary to that of a target gene.

Specifically, the left-side probe part or the right-side probe part may be an oligonucleotide comprising 15-150 nucleotide sequences.

Specifically, the nucleotide sequence of the left-side probe part may be in 5′→3′ direction from top to top, and the nucleotide sequence of the right-side probe part may be in 5′→3′ direction from bottom to top.

Specifically, the linker part may comprise amino-modified dideoxythymidine such as C6dT, C3dT, C12dT or C18dT for binding to an aldehyde-coated solid support.

Specifically, the probe of the present disclosure may comprise a peptide nucleic acid (PNA).

Specifically, the probe of the present disclosure may be prepared by a synthesis method comprising 1) a detritylation step, 2) a coupling step, 3) a capping step and 4) an oxidation step.

Specifically, the left-side probe part and the right-side probe part may respectively comprise oligonucleotides having nucleotide sequences complementary to two different regions of a target gene.

Specifically, the left-side probe part and the right-side probe part may respectively comprise oligonucleotides having nucleotide sequences complementary to the same region of a target gene.

Specifically, the left-side probe part and the right-side probe part may respectively comprise oligonucleotides having nucleotide sequences complementary to those of different target genes.

Specifically, one of the left-side probe part and the right-side probe part may comprise an oligonucleotide having a nucleotide sequence complementary to that of a target gene and the other probe part may comprise an oligonucleotide having a nucleotide sequence complementary to that of a control gene.

Specifically, the control gene may lack complementarity to the target gene and be nonexistent or unexpressed in the sample.

Specifically, the control gene may be the motD gene of E. coli.

Specifically, the probe of the present disclosure may be an oligonucleotide having at least one nucleotide sequence selected from SEQ ID NOS 5-50.

In another aspect, the present disclosure provides a DNA microarray comprising the probe directly spotted on a solid support.

Specifically, the solid support may be selected from a group consisting of a glass slide, a bead, a microplate well, a silicon wafer and a nylon membrane.

Specifically, the human beta-globin gene may be further spotted on the DNA microarray.

Specifically, the DNA microarray may have 8 wells for spotting the probe.

Specifically, the probe may comprise an oligonucleotide having at least one nucleotide sequence selected from SEQ ID NOS 5-50 and may be for detecting and genotyping HPV.

Specifically, the probe may bind complementarily to an oligonucleotide primer having a nucleotide sequence of SEQ ID NO 4 with the 5′-terminal labeled with Cy5 and an oligonucleotide primer having a nucleotide sequence of SEQ ID NO 1 with the 5′-terminal labeled with Cy3.

Specifically, the probe may comprise an oligonucleotide having a nucleotide sequence selected from SEQ ID NOS 51-55 and may be for detecting and genotyping Neisseria gonorrhoeae (NG), Chlamydia trachomatis (CT), herpes simplex virus (HSV), Treponema pallidum (TP) and Haemophilus ducreyi (HD) as a pathogen causing a sexually transmitted disease (STD), respectively.

Specifically, the probe may comprise an oligonucleotide having a nucleotide sequence selected from SEQ ID NOS 56-199 and may be for detecting and genotyping influenza virus.

Specifically, the probe may comprise an oligonucleotide having a nucleotide sequence selected from SEQ ID NOS 212-213 and may be for analyzing expression of the β-actin or epidermal growth factor receptor (EGFR) gene.

Specifically, one of the left-side probe part and the right-side probe part of the probe may comprise an oligonucleotide complementary to the single nucleotide polymorphism (SNP) site of a sense strand of a target nucleic acid and the other may comprise an oligonucleotide complementary to an antisense strand of the target nucleic acid lacking the SNP site, and the probe may be for analyzing SNP.

Specifically, the probe may comprise an oligonucleotide having at least one nucleotide sequence selected from SEQ ID NOS 220-239 and may be for analyzing SNP of the ACE, ADRB2, Apo E, CETP, CFH, ESR1, IL1A, MTHFR or NOS3 gene.

Specifically, the probe may comprise an oligonucleotide having at least one nucleotide sequence selected from SEQ ID NOS 258-272 and may be for analyzing mutation of the K-ras gene.

Specifically, the right-side probe part of the d-shaped probe of the present disclosure may comprise an oligonucleotide having a nucleotide sequence complementary to point mutation of A, C, G or T. In this case, the nucleotide complementary to the point mutation may be located at the center of the right-side probe part and the right-side probe part may be 15-30 bp in length. The d-shaped probe may be for analyzing point mutation.

In another general aspect, the present disclosure provides a kit for genetic analysis of a sample comprising the DNA microarray, a primer set and a buffer for PCR of a target gene in the sample, and a hybridization buffer.

Specifically, the primer set for PCR may be an oligonucleotide having a nucleotide sequence selected from SEQ ID NOS 208-211 for amplification of the gene of influenza A virus.

Specifically, the primer set for PCR may be an oligonucleotide having a nucleotide sequence of SEQ ID NOS 214-219 for quantitative real-time PCR of the β-actin and EGFR genes.

Specifically, the primer set for PCR may be an oligonucleotide having at least two nucleotide sequences selected from SEQ ID NOS 240-257 for detection of SNP.

Specifically, the kit of the present disclosure may be for diagnosis, prevention, prognosis or personalized therapy of a disease.

In another general aspect, the present disclosure provides a genetic analysis method comprising placing a target nucleic acid of a sample labeled with a label on the DNA microarray and hybridizing the probe of the DNA microarray with the target nucleic acid.

Specifically, the label may be at least one selected from a group consisting of Cy3, Cy5, Cy5.5, BODIPY, Alexa 488, Alexa 532, Alexa 546, Alexa 568, Alexa 594, Alexa 660, rhodamine, TAMRA, FAM, FITC, Fluor X, ROX, Texas Red, Orange Green 488X, Orange green 514X, HEX, TET, JOE, Oyster 556, Oyster 645, BODIPY 630/650, BODIPY 650/665, Cal Fluor Orange 546, Cal Fluor Red 610, Quasar 670 and biotin.

Specifically, the target nucleic acid may be labeled with the label by PCR, RT-PCR or in vitro transcription.

Specifically, the genetic analysis method may further comprise, after the hybridization, investigating the expression level of the target nucleic acid by analyzing a signal from the label using a fluorescence scanner.

Specifically, the signal may be analyzed through normalization.

Specifically, the normalization may comprise a triple normalization procedure of investigating Cy5 and Cy3 signals from each spot excluding the background noise signal and comparing with the Cy3 signal from the β-actin gene as a housekeeping gene.

Specifically, the target nucleic acid may be selected from a group consisting of DNA, RNA, cDNA and cRNA.

Specifically, the cDNA may be labeled with Cy3 by RT-PCT and the cRNA may be labeled with Cy3 by in vitro transcription.

Specifically, the cDNA or cRNA labeled with Cy3 may be mixed with the motD gene of E. coli labeled with Cy5 as an external control and the resulting mixture may be hybridized.

In another general aspect, the present disclosure provides a clinical diagnosis method using the Y-shaped probe, comprising:

a first step of designing the Y-shaped probe;

a second step of synthesizing the Y-shaped probe;

a third step of fabricating a DNA microarray using the Y-shaped probe;

a fourth step of preparing a nucleic acid sample to be placed on the DNA microarray and labeling it with a labeling dye by PCR, in vitro transcription, or the like;

a fifth step of placing the on the DNA microarray and performing hybridization;

a sixth step of reading and analyzing signals after the hybridization;

a seventh step of detecting the presence and amount of a target gene using the Y-shaped probe;

an eighth step of analyzing various genotypes using the Y-shaped probe and applying the result for clinical practice, specifically detecting HPV, influenza or a pathogen causing sexually transmitted infection and identifying its type, thereby diagnosing the disease and determining the therapeutic regimen;

a ninth step of analyzing the expression level of multiple genes using the Y-shaped probe;

a tenth step of analyzing the mutation of a specific nucleotide sequence, e.g. SNP, point mutation, etc., using the Y-shaped probe; and

an eleventh step of applying for clinical diagnosis, specifically predicting the outbreak of a disease through SNP analysis and preventing the disease, selecting a personalized drug by predicting drug efficacy and side effect through SNP analysis, or screening or diagnosing a disease through analysis of mutation or gene expression.

### Advantageous Effects

The Y-shaped probe of the present disclosure, the variant thereof, and the DNA microarray for genetic analysis using the same allow accurate analysis of the presence of a specific gene and its type and the change in expression level and nucleotide sequence. Furthermore, they are clinically very useful since they allow fast and accurate diagnosis of various infections and diseases including cancer, prediction of disease severity and prognosis, determination of therapeutic regimen, selection of personalized drugs, or the like.

### DESCRIPTION OF DRAWINGS

FIG. 1 schematically shows a Y-shaped probe according to an exemplary embodiment of the present disclosure.

FIG. 2 shows a chemical structure of an internal amino modifier C6 dT (iAmMC6) which is used to bind the Y-shaped probe of the present disclosure on the surface of a chip.

FIG. 3 shows a result of amplifying the cervical cancer-causing HPV and human beta globin (HBB) genes by PCR and electrophoresing them (Example 5). The HPV-16 L1 gene was labeled with Cy5 and the HBB gene was labeled with Cy3. DNA was extracted from Caski cells (reference for HPV-16) according to a known method. PCR was performed using the primers of the L1 gene and the HBB gene shown in Table 1 and electrophoresis was performed on 0.8% agarose gel. Lane M: 100 bp size marker, lane 1: negative control, lane 2: PCR product of HPV-16 L1 gene (185 bp), lane 3: PCR product of HBB gene (102 bp).

FIG. 4 shows grids occurring in each well of a DNA biochip capable of diagnosing the cervical cancer-causing virus HPV (Example 4). The red area indicates high-risk type HPV, the green area indicates low-risk type HPV, the yellow area indicates the HBB gene, and the azure area indicates YP16S and YP16AS as one of the Y-shaped probe of the present disclosure.

FIG. 5 shows a result of spotting the Y-shaped probe of the present disclosure simultaneously on 22 HPV chips fabricated using the grids of FIG. 4 and hybridizing with HPV-16 (labeled with Cy5) and HBB (labeled with Cy3) (Example 5). Well 1 & 2: sample labeled with HPV 16-Cy5 & HBB-Cy5, well 3 & 4: sample labeled with HBB-Cy5, well 5 & 6: sample labeled with HPV 16-Cy5 & HBB forward primer-Cy3, well 7 & 8: sample labeled with HPV 16-Cy5 & HBB reverse primer-Cy3.

FIG. 6 shows a result of scanning only one well of a chip hybridized with the HBB forward primer-Cy3 PCR product at 532 nm (Example 6).

FIG. 7 shows a result of electrophoresing a PCR product obtained using a reference for an STD chip on 3% agarose gel. M: 100 bp DNA size marker, lanes 1-6: PCR product of Haemophilus ducreyi (440 bp), PCR product of herpes simplex virus 1 (384 bp), PCR product of herpes simplex virus 2 (400 bp), PCR product of Chlamydia trachomatis (321 bp), PCR product of Neisseria gonorrhoeae (284 bp) and PCR product of Treponema pallidum (260 bp), respectively, lane 7: multiplex PCR product obtained using 5 references in Example 9 (It can be seen that all the 5 genes were amplified by PCR).

FIG. 8 shows a result of hybridizing the Y-shaped probe with Neisseria gonorrhoeae on an STD chip (Example 9).

FIG. 9 shows a result of hybridizing the Y-shaped probe with Chlamydia trachomatis on an STD chip (Example 9).

FIG. 10 shows a result of hybridizing the Y-shaped probe with Treponema pallidum on an STD chip (Example 9).

FIG. 11 shows a result of hybridizing the Y-shaped probe with Haemophilus ducreyi on an STD chip (Example 9).

FIG. 12 shows a result of hybridizing the Y-shaped probe with herpes simplex virus on an STD chip (Example 9).

FIG. 13 shows grids occurring in each well of an influenza virus A DNA chip using the Y-shaped probe (Example 10).

FIG. 14 shows a result of hybridization on an influenza virus A chip using a reference (Example 10). The H gene was labeled with Cy5, the N gene was labeled with Cy3, and the RPP, SWH1, SW infA and infA genes were labeled with Cy5. The first photograph is an image obtained by scanning at both 532 nm and 635 nm using the Y-shaped probe of the present disclosure. The swine influenza virus (H1N1) showed signals only at the H1N1, H10N1, infA, RPP, swH1 and swinfA spots of chip of the present disclosure. At 635 nm, signal was observed only at the N1 gene. And, at 532 nm, signals were observed only at the H1N1, infA, RPP, swH1 and swinfA spots. Accordingly, it was confirmed that the Y-shaped probe of the chip of the present disclosure is hybridized with the gene of swine influenza virus.

FIG. 15a shows a result of performing one-step real-time RT-PCR of the RNase P, SWH1, SW infA and infA genes using the TaqMan probe and analyzing using the Rotorgene 6.0 software. Real-time RT-PCR was performed using nc (negative control), pc (positive control; novel influenza-positive viral RNA) and RNA extracted from patient samples. The three patient samples were found to be negative since they were detected only at the RNase P gene, whereas pc was detected in all of the SWH1, SW infA, infA and RNase P genes.

FIG. 15b shows a result of analyzing only the RNase P and SWH1 genes of seven clinical samples using the TaqMan probe. Among the seven samples, only two samples were found to be positive with SWH1 and RNase P detected Four samples were found to be negative since amplification occurred only at the RNase P gene and the remaining one sample was tested again since even the amplification of the RNase P gene was not detected.

FIG. 16 shows a result of electrophoresing the real-time RT-PCR products of the RNase P gene and the SWH1 gene on 2% agarose gel. As can be seen from the images, test using the DNA chip of the present disclosure or by real-time RT-PCR is necessary for the H1N1 gene since it is difficult to distinguish positive and negative results only with the size of the PCR products. M: 100 bp DNA size marker, N: negative control, lanes 1-6: PCR products from patient samples, cDNA: cDNA of novel influenza-positive control.

FIG. 17 schematically shows a basic structure of a Y-shaped probe for testing gene expression according to the present disclosure as well as hybridization of a sample with the cRNA of a control substance on a microarray on which the probe is spotted.

FIG. 18 schematically shows an external control substance for analysis of gene expression using the Y-shaped probe. Specifically, the sequences of synthetic oligonucleotide (A) and plasmid (B) comprising the T7 promoter, poly-A tail, and E. coli motD gene used in Example 11 are shown. They are used as templates to prepare a target labeled with Cy-5 by through in vitro transcription, which is hybridized with cRNA obtained from the sample on the DNA microarray.

FIG. 19 shows a result of extracting RNA from the samples of a healthy person and a patient, synthesizing cDNA therefrom, and analyzing expression of the EGFR gene and the β-actin gene using the Y-shaped probe microarray (Example 11).

FIG. 20 shows a result of extracting RNA from the samples of a healthy person and a patient, synthesizing cDNA therefrom, and analyzing expression of the EGFR gene and the β-actin gene by qRT-PCR (Example 11). Whereas the Ct value of the β-actin gene was almost the same in the two samples, the EGFR gene was expressed in the patient sample but not in the healthy person sample.

FIG. 21 shows a result of genetic testing using an SNP genotyping chip comprising the Y-shaped probe. The image was obtained using a two-color fluorescence scanner. For each spot, normalized signal of Cy-3 with respect to Cy-5 was investigated after removal of the background signal, and the probe perfectly matching the spot was found based thereon. As a result, unfavorable (high-risk) SNP was detected for the CFH, CETP and MTHFR genes. That is to say, the reporter gene obtained by hybridization with the Cy3-labeled PCR product of each gene looks green when SNP is present, whereas the reference gene hybridized with the Cy5-labeled PCR product looks red because it is free of SNP. Accordingly, a gene lacking the SNP site exhibits red color of Cy5 whereas one having the SNP site exhibits a color complementary thereto. In the present sample, SNP (Y402H, rs1061170) was found at the 402nd codon of the complement factor H (CFH) gene, and SNP (G1533A) was found at the 1553rd nucleotide of the cholesteryl ester transfer protein (CETP) gene. Also, SNP (C677T, Ala222Val) was found at the 677th nucleotide of the methylenetetrahydrofolate reductase (MTHFR) gene.

FIG. 22 schematically shows d-shaped probes for GTT (Gly) and AGT (Ser) at the 12nd codon of the K-ras gene.

FIG. 23 shows a scanning result of the K-ras DNA microarray. Analysis of the blood sample from a lung cancer patient revealed mutation of the 12nd codon of the K-ras gene from GTT to AGT (Gly12Ser).

### MODE FOR INVENTION

The present disclosure will be described in further detail through examples. However, the following examples are for illustrative purposes only and not intended to limit the scope of the present disclosure.

### Example 1

### Designing of Y-Shaped Probe

The most fundamental part of DNA chip development is the designing of the Y-shaped duplex oligonucleotide probe of the present disclosure. This includes attaching of a linker for placing the probe on a solid support as well as attaching of a spacer and labeling with a labeling dye for detection of signal.

The Y-shaped probe of the present disclosure looks like a tree having two branches of different oligonucleotide probe parts stemming from one stem. The root of the tree fixing the probe on a support such as a glass slide is called a linker or a spacer. When a sample falls on the tree like snow and DNA or RNA having a sequence complementary to that of one of the two probe parts binds specifically thereto, hybridization occurs and the remaining snow is washed away. The hybridization product is labeled with a label (labeling dye) to read signals thereof.

Unlike the so-called molecular beacon or hairpin probe reported in the literature, the Y-shaped probe of the present disclosure lacks a loop portion and no quencher is used (Wang K, Tang Z, Yang C J, Kim Y, Fang X, Li W, Wu Y, Medley C D, Cao Z and Li J. Molecular engineering of DNA: Molecular beacon. Angew Chem Int Ed Engl. 2009; 48(45): 856-870; Li Y, Zhou X and Ye D. Molecular beacons: an optimal multifunctional biological role. Biochemical and Biophysical Research Communication. 2008; 373: 457-461; Yao G Y and Tan W. Molecular-beacon-based array for sensitive DNA analysis. Analytical Biochemistry. 2004; 331: 216-223; Broude N E. Stem-loop oligonucleotides: a robust tool for molecular biology and biotechnology. Trends in Biotechnology. 2002; 20(6): 249-256). Accordingly, the probe of the present disclosure is entirely different from the molecular beacon. Also, it is quite different in structure or operation from other probes modified from the molecular beacon published in the literature (Tsourkas A, Behlke M A and Bao G. Structure-function relationship of shared stem and conventional molecular beacons. Nucleic Acids Research. 2002; 30(19): 4208-4215; Misra A, Kumar P and Gupta K C. Design and Synthesis of hairpin probe for specific mis-match discrimination. Nucleic Acids Symposium Series. 2007; 51: 311-312; Riccelli R V, Merante F, Leung K T, Bortolin S, Zastawny R L, Janeczko R and Benight A S. Nucleic Acids Research. 2001; 29(4): 996-1004).

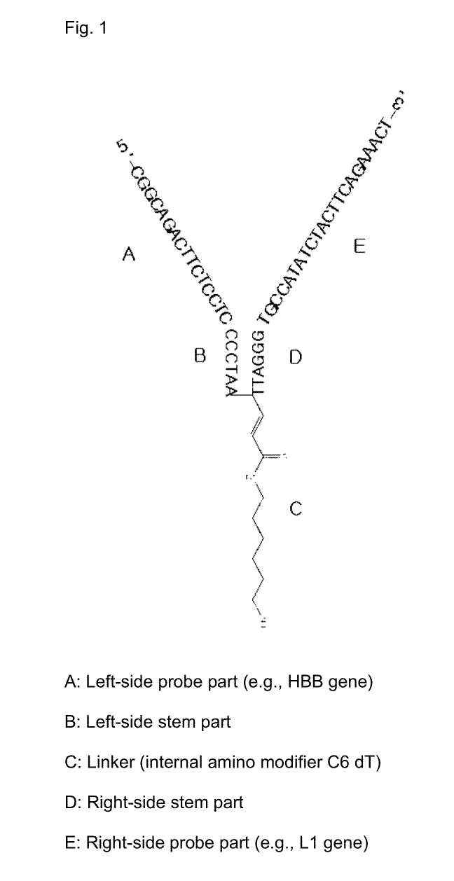

As seen from FIG. 1, the Y-shaped probe of the present disclosure comprises, in 5′→3′ direction, from left top to right top, (1) a left-side probe part (A), (2) a left-side stem part (B), (3) a linker or spacer part (C), (4) a right-side stem part (D) and (5) a right-side probe part (E).

The structure of each portion will be described in detail hereinafter.

(1) Stem Part

For the Y-shaped probe of the present disclosure to be firmly established, the stem part should be formed adequately. The stem part comprises two oligonucleotides having complementary sequences bound to each other. For tight binding, each of the left-side stem part and the right-side stem part may comprise the nucleotide G in at least half of the entire nucleotide sequence, with the nucleotide T or A inserted therebetween. For example, it may comprise GnTGmTGo. Although various nucleotide sequences can be used, naturally occurring ones are desired. A telomere consisting of repetitive nucleotide sequences exists at the end of the chromosome of eukaryotic organisms. The repetitive sequences are TTAGGG, TTTAGGG or T1-3(T/A)G3- for mammals including human and TTGGGG or TTTTGGGG for other organisms. A similar structure occurs at the switch portion of immunoglobulin (Balagurumoothy P, Brahmachari S K, Mohnaty D, Bansal M and Sasisekharan V. Hairpin and parallel quartet structures for telomeric sequences. Nucleic Acids Research. 1992; 20(15): 4061-4067; Balagurumoothy P and Brahmachari S K. Structure and stability of human telomeric sequence. Journal of Biochemistry. 1994; 269(34): 21858-21869).

Specifically, each of the left-side stem part and the right-side stem part may comprise at least one repeating nucleotide unit selected from the followings.

e.g.)

| | |
|---|---|
| 1. | TTGGG |
| 2. | TAGGG |
| 3. | TTGGGG |
| 4. | TTTGGG |
| 5. | TTAGGG |
| 6. | TTTGGGG |
| 7. | TTTAGGG |
| 8. | TTTTGGGG |
| 9. | TTTAGGGG |

That is to say, 5-9 oligonucleotides may bind complementarily, and the number of the oligonucleotides can be increased further. In terms of cost and efficiency, the human telomere comprising the nucleotide sequence TTAGGG-AATCCC may be used as the repeating unit. However, the length can be changed variously. Typically, the length may be C6, C12 or C18.

(2) Left-Side and Right-Side Probe Parts

The oligonucleotide probe is designed to be complementary to a target gene. Any nucleotide sequence is possible, but the oligonucleotide sequence and length of the left-side and right-side probe parts should be adequately designed. The probe parts should be selected such that the left-side and right-side oligonucleotides are not complementary to each other so as to bind with each other and such that each of them does not form a secondary structure.

Direction is also important in the designing of the Y-shaped probe. The left-side probe part (A) comprises a sequence in reverse (3′→5′) direction and the right-side probe part (E) comprises a sequence in forward (5′→3′) direction.

Specifically, each of the left-side and right-side probe parts may be 15-75 bp in length, but the length may be increased to about 150 bp or decreased to shorter than 15 bp depending on situations. The precise length of each probe part may vary depending on the purpose of test, structure and nucleotide sequence of the target gene, sensitivity, specificity and reproducibility of test, noise, and how to set the bias. When higher specificity is desired, a short length of usually 15-25 bp is selected. When focus is placed on sensitivity, a long length of usually 40-70 bp is selected. For analysis of allele-specific hybridization to investigate SNP or mutation, the probe length is set to about 15-22 bp and it is designed such that the difference in one, two or three nucleotides at the center portion is discernible. When the sample is a PCR product of a specific gene and when detection of a specific nucleotide sequence and genotyping of the product are desired, for example for analysis of species and subspecies of virus or bacteria causing infection, the probe length is set to about 20 bp and it is designed such that the difference in at least three nucleotides at the center portion is discernible. Some sequences may not be appropriate to use.

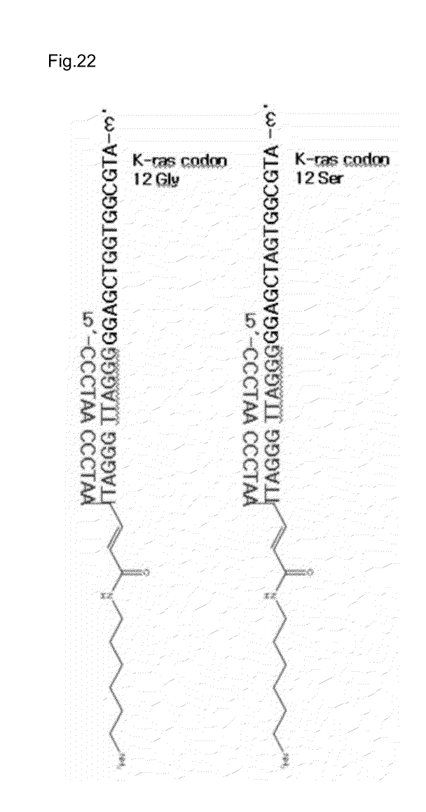

The left-side and right-side probe parts need not be symmetrical in length. Depending on purposes and applications, the length of the left-side probe part may be decreased extremely as in FIG. 22 to form a d-shaped probe. Also, the right-side probe part may be decreased extremely to form a b-shaped probe.

The nucleotide sequence and the length of the oligonucleotide probe can be determined according to a known method. That is to say, from the target gene to be tested, the region with the least complementarity to the non-targeting gene is selected. Then, the probes should be designed such that the melting temperature (Tₘ) of the probes is within an appropriate range considering the hybridization temperature. Of course, the percentage of C+G and the probe length should be considered. Care should be taken such that a secondary structure is not formed and it is desired to analyze the self-folding energy. After selecting a set of candidate probes using a sliding window, the probe most likely to be hybridized is finally selected from the candidate probes considering the complementary binding with the target gene and various other conditions. The optimum probe may also be selected using a virtual hybridization module. Since the designing of the probe set can be seen as optimization of finding the sequence most likely to be hybridized, the evolutionary computation technique is often used. Also, learning techniques such as artificial neural network are employed (David P. Kreil, Roslin R. Russell and Steven Russell. Microarray Oligonucleotide Probes. Methods in Enzymology 2006; 410: 73-98; Lemoline S, Combes F and Le Crom S. An evaluation of custom microarray application: the oligonucleotide design challenge. Nucleic Acids Research. 2009; 37(6): 1726-1739).

Simply, a commercially available oligonucleotide probe design program such as ArrayOligoSelector, CommOligo, HPD, Mprime, OliD, OligoArray, OLigodb, OLigoFaktory, OLigoPicker, POligoWiz, Oliz, Ospery, PICKY, PROBEmer, Probesel, ProbeSelect, ROSO, SEPON, YODA, etc. may be used. They support most of the necessary fundamental information including cross hybridization analysis, analysis of optimum number of probes, avoidance of the so-called low-complexity zone, directional setup, or the like (Bozdech Z, Zhu J, Joachimiak M P, Cohen F E, Pulliam B, DeRisi J L. Expression profiling of the schizont and trophozoite stages of Plasmodium falciparum with a long-oligonucleotide microarray. Genome Biol. 2003; 4: R9; Li X, He Z, Zhou J. Selection of optimal oligonucleotide probes for microarrays using multiple criteria, global alignment and parameter estimation. Nucleic Acids Res. 2005; 33: 6114-6123; Rimour S, Hill D, Militon C, Peyret P. GoArrays: highly dynamic and efficient microarray probe design. Bioinformatics. 2005; 21: 1094-1103; Chung W H, Rhee S K, Wan X F, Bae J W, Quan Z X, Park Y H. Design of long oligonucleotide probes for functional gene detection in a microbial community. Bioinformatics. 2005; 21: 4092-4100; Rouchka E C, Khalyfa A, Cooper N G. MPrime: efficient large scale multiple primer and oligonucleotide design for customized gene microarrays. BMC Bioinformatics. 2005; 6: 175; Talla E, Tekaia F, Brino L, Dujon B. A novel design of whole-genome microarray probes for Saccharomyces cerevisiae which minimizes cross-hybridization. BMC Genomics. 2003; 4: 38; Rouillard J M, Zuker M, Gulari E. OligoArray 2.0: design of oligonucleotide probes for DNA microarrays using a thermodynamic approach. Nucleic Acids Res. 2003; 31: 3057-3062; Mrowka R, Schuchhardt J, Gille C. Oligodb-interactive design of oligo DNA for transcription profiling of human genes. Bioinformatics. 2002; 18: 1686-1687; Schretter C, Milinkovitch M C. OligoFaktory: a visual tool for interactive oligonucleotide design. Bioinformatics. 2006; 22: 115-116; Wang X, Seed B. Selection of oligonucleotide probes for protein coding sequences. Bioinformatics. 2003; 19: 796-802; Wernersson R, Nielsen H B. OligoWiz 2.0—integrating sequence feature annotation into the design of microarray probes. Nucleic Acids Res. 2005; 33: W611-W61; Chen H, Sharp B M. Oliz, a suite of Perl scripts that assist in the design of microarrays using 50mer oligonucleotides from the 3′ untranslated region. BMC Bioinformatics. 2002; 3: 27; Gordon P M, Sensen C W. Osprey: a comprehensive tool employing novel methods for the design of oligonucleotides for DNA sequencing and microarrays. Nucleic Acids Res. 2004; 32: e133; Chou H H, Hsia A P, Mooney D L, Schnable P S. Picky: oligo microarray design for large genomes. Bioinformatics. 2004; 20: 2893-2902; Emrich S J, Lowe M, Delcher A L. PROBEmer: a web-based software tool for selecting optimal DNA oligos. Nucleic Acids Res. 2003; 31: 3746-3750; Kaderali L, Schliep A. Selecting signature oligonucleotides to identify organisms using DNA arrays. Bioinformatics. 2002; 18: 1340-1349; Li F, Stormo G D. Selection of optimal DNA oligos for gene expression arrays. Bioinformatics. 2001; 17: 1067-1076; Reymond N, Charles H, Duret L, Calevro F, Beslon G, Fayard J M. ROSO: optimizing oligonucleotide probes for microarrays. Bioinformatics. 2004; 20: 271-273; Hornshoj H, Stengaard H, Panitz F, Bendixen C. SEPON, a Selection and Evaluation Pipeline for OligoNucleotides based on ESTs with a non-target Tₘ algorithm for reducing cross-hybridization in microarray gene expression experiments. Bioinformatics. 2004; 20: 428-429; Nordberg E K. YODA: selecting signature oligonucleotides. Bioinformatics. 2005; 21: 1365-1370).

In the Y-shaped probe of the present disclosure and the d-shaped probe which is a variant thereof, various combinations of the left-side and right-side probe parts may be considered. Representative combinations include the followings.

1) The probe parts of the Y-shaped probe of the present disclosure may be designed by selecting a target gene from the sample and then selecting two different regions in the same gene. Since this allows dual detection for one gene, sensitivity can be improved over the existing probe which allows only one testing. For example, pathogens causing sexually transmitted infection can be diagnosed more accurately using the Y-shaped probe as described in Example 9.

2) The probe parts of the Y-shaped probe of the present disclosure may be designed by selecting two target genes from the sample. Since this allows dual detection of two genes for one disease, accuracy can be improved over the existing probe which allows only one testing for one gene. As a result, the testing becomes easier and cost is reduced. For example, for accurate genotyping of influenza, both the hemagglutinin and neuraminidase genes should be tested. As described in Example 10, the two genes can be diagnosed more easily and more conveniently using the Y-shaped probe.

3) The probe parts of the Y-shaped probe of the present disclosure may be designed by selecting a target gene for one (e.g., left-side) probe part and selecting a control gene for the other (e.g., right-side) probe part. For example, when genotyping HPV, the presence and accurate genotype of HPV can be analyzed accurately while avoiding false positive or false negative result by using a probe specific for HPV subtype in the L1 gene as one probe part of the Y-shaped probe and using a probe specific for the internal control or reference gene existing in all human sample as the other probe part, as described in Examples 3-8. Alternatively, a probe specific for HPV subtype in the L1 gene may be used as one probe part of the Y-shaped probe and a probe binding with all HPV subtypes in the L2 gene may be used as the other probe part. Alternatively, a probe specific for HPV subtype in the L1 gene may be used as one probe part of the Y-shaped probe and a probe specific for HPV subtype in the E6/E7 L2 gene may be used as the other probe part. The resultant novel HPV microarray may be greatly helpful in diagnosis of HPV infection and early diagnosis of cervical cancer, anal cancer, head and neck cancer, etc.

4) The probe parts of the Y-shaped probe of the present disclosure may be designed by using a probe for the target gene to be tested as one probe part and using a probe for a housekeeping gene as the other probe part. Then, after labeling the target and control housekeeping genes differently with Cy-3 and Cy-5, hybridization is performed on the microarray by reverse transcription polymerase chain reaction (RT-PCR). Then, after investigating signals from Cy-3 and Cy-5 for each spot excluding the background noise signal and analyzing them through normalization, the signal ratio of the target gene to the housekeeping gene (Cy3/Cy5) can be calculated for each spot. And, by averaging the result for several spots, relative expression of the target gene can be analyzed statistically.

5) The Y-shaped probe of the present disclosure may be used for simultaneous analysis of multiple genes. For example, as described in Example 11, probes for multiple target genes to be tested are spotted on one probe part of the Y-shaped probe and a probe for an internal control gene is spotted on the other probe part to fabricate a microarray. Then, two samples are prepared. One is a cRNA sample of the genes to be tested, which is labeled with a fluorescent dye (e.g., Cy-3) by in vitro transcription. Separately from this, a cRNA sample of the internal control gene labeled with a fluorescent dye (e.g., Cy-5) by in vitro transcription is prepared. The two samples, i.e. the cRNA sample of the genes to be tested and the cRNA sample of the control gene are mixed and hybridized on the microarray. Subsequently, after investigating signals from Cy-3 and Cy-5 for each spot excluding the background noise signal and analyzing them through normalization, the signal ratio of the target genes to the control gene (Cy3/Cy5) can be calculated for each spot. And, by averaging the result for several spots, relative expression of the multiple target genes can be analyzed statistically. In this manner, high-throughput analysis of expression of all known human genes is possible theoretically. As described in Example 11, this method may be used to investigate the expression of epidermal growth factor receptor (EGFR) in cancer patient. This may lead to refined anticancer therapy through establishment of the standard for administration of blocking drug or antibody drug (Ellis L M and Hicklin D J. Resistance to targeted therapies: refining anticancer therapy in the era of molecular oncology. Clinical Cancer Research. 2009; 15(24): 7471-7478).

6) A probe for the SNP site of the sense strand of a target gene may be used as the left-side probe part of the Y-shaped probe of the present disclosure and a control probe lacking the SNP site of the antisense strand of the target gene may be used as the right-side probe part. Probes specific for the wild- or normal-type and mutant-type genes are spotted on the left-side probe part. The probes are about 15-30 bp in length. Then, the sense strand of the target gene is labeled with Cy-3, and the antisense strand is labeled with Cy-5. After performing PCR, the resulting product is hybridized on the microarray. Then, after investigating signals from Cy-3 and Cy-5 for each spot excluding the background noise signal and analyzing them through normalization, the probe perfectly matching the spot is detected. In this manner, it is possible to identify whether the gene is wild type or mutant type as well as the heterozygosity. As described in Example 12, if the mutant-type complement factor-H gene (Y402H) is identified in SNP testing, there is a high risk of age-related macular degeneration (AMD). To prevent the disease, eating a lot of vegetables having antioxidative functions, quitting smoking and wearing sunglasses on sunny days may be recommended. That is to say, SNP testing using the DNA microarray of the present disclosure is helpful in predicting and preventing diseases.

7) The variant of the Y-shaped probe of the present disclosure may be used for detecting mutation. A probe specific for the mutation site of a target gene to be tested is spotted on the right-side probe part of the Y-shaped probe and the left-side probe part is reduced greatly to prepare a d-shaped probe. Probes capable of detecting A, C, G and T nucleotides of the mutation site are prepared. The nucleotide of the mutation site is located at the center of the probe. The probe may be about 15-25 bp in length. After labeling the target gene identically with Cy-3 or Cy-5, followed by hybridization, the probe of the spot that matches perfectly is detected. In this manner, it can be identified whether the nucleotide sequence to be tested for mutation is A, C, G or T. As described in Example 13, this method may be used to detect mutation of the K-RAS gene. This may be helpful in diagnosis of lung cancer. A poor prognosis of a lung cancer patient can be predicted and the patient may be recommended to avoid EGFR blocking drug or antibody drug since they exhibit high drug resistance (Ellis L M and Hicklin D J. Resistance to targeted therapies: refining anticancer therapy in the era of molecular oncology. Clinical Cancer Research. 2009; 15(24): 7471-7478). That is to say, mutation testing using the DNA microarray of the present disclosure may be helpful in diagnosis and prognosis evaluation of diseases and determination of therapeutic regimen.

As described above, the Y-shaped probe of the present disclosure can be modified variously and may be used in nearly any genetic testing.

(3) Linker (Spacer) Part

The Y-shaped probe of the present disclosure may be spotted on various (solid) supports. For example, the support may be a glass slide, a bead (X-MAP microsphere), a microplate well, a silicon wafer, a membrane, or the like. In terms of economy and easiness of operation and based on various attempts and experiences, spotting on a glass slide whose surface is activated by special treatment is considered preferentially. A terminal, uncharged amphiphilic linker or spacer having a plurality of carbon groups is connected to the Y-shaped probe and attached on a slide. Hybridization may not occur well due to electrostatic effect if the probe is attached on the support without the linker (spacer) (Keril D P, Russell R R and Russell S. Microarray oligonucleotide probes. Methods in Enzymology. 2006; 410: 73-98).

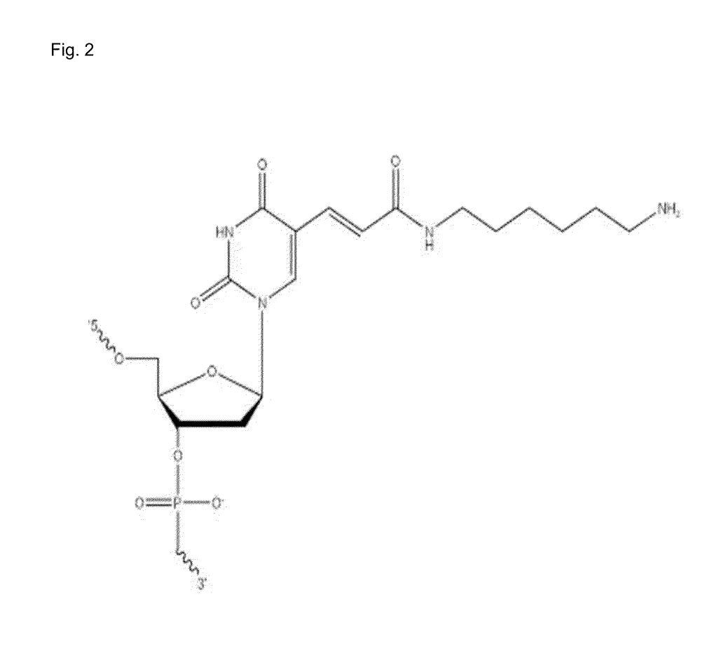

In the present disclosure, amino-modified dideoxythymidine (internal amino modifier CndT; iAmMCnT) with n ranging from 3 to 60 is inserted. In terms of economic efficiency, short iAmMC6T having 6 carbons may be used. At the 5′-terminal of iAmMC6dT, the modified C6 amine linker of the left-side stem part binds with the aldehyde group on the glass slide surface. The nucleotide A of the 3′-terminal left-side stem part binds with the nucleotide T of the 5′-terminal of the right-side stem part. The Y-shaped probe may be fixed on a chip via binding to the ribose of the iAmMC6dT. As a representative example, the chemical structure of iAmMC6T is shown in FIG. 2. Also, iAmMCnT with n being 3, 12, 18, 24, etc. may be used.

(4) Label

Various known substances may be used as the label of the probe. For example, any of Cy5, BODIPY Cy3, Alexa 532, Alexa 546, Rodamin, TAMRA, FAM, FITC, Fluor X, Alexa 488, Alexa 568, ROX, Texas Red and Alexa 594 may be used. In addition, labeling can be achieved using the binding between biotin and avidin (streptavidin). For example, the terminal of the primer used for performing PCR is labeled with biotin or biotin-labeled dNTPs are used for PCR. The PCR product may be detected using fluorescence-labeled avidin (streptavidin). Besides, nanoparticles such as Au or Ag nanoparticles may be used for labeling.

In the present disclosure, instead of attaching a label to the Y-shaped probe, the sample nucleic acid is labeled and hybridized with the Y-shaped probe on the DNA microarray. However, depending on purposes and applications, a label may be directly attached to the Y-shaped probe and then the Y-shaped probe may be reacted with the sample nucleic acid. In this case, the label is usually attached at the 3′-terminal of the right-side probe part. However, it may also be attached at the 5′-terminal of the left-side probe part and at both the 3′-terminal of the right-side probe part and the 5′-terminal of the left-side probe part. Also, it may be attached inside the probe, not at the terminals of the probe. Various known labels may be used as the label of the probe. For example, any of Cy5, Bodipsy, Cy3, Alexa 532, Alexa 546, Rodamin, TAMRA, FAM, FITC, Fluor X, Alexa 488, Alexa 568, ROX, Texas Red and Alexa 594 may be used.

### Example 2

### Synthesis of Y-Shaped Probe

The Y-shaped probe designed in Example 1 may be synthesized as follows. The Y-shaped oligonucleotide probe is synthesized through a cycle of 1) detritylation (removal of DMT), 2) coupling, 3) capping and 4) oxidation. One nucleotide is bound through each cycle. Accordingly, a desired oligomer may be synthesized by reacting dA, dG, dT and dC in the order of the desired nucleotide sequence. After the synthesis is completed, ammonium hydroxide is added to separate the oligomer from the support (deprotection). The 3′-terminal of the oligonucleotide is fixed to the solid support and synthesis is carried out in a column. Accordingly, the synthesis occurs in 3′→5′ direction. Controlled pore glass (CPG) or polystyrene is used as the support. Highly hydrophobic polystyrene provides better synthesis efficiency than CPG. A stationary nucleoside has a free 5′-terminal protected by the dimethoxytrityl (DMT) group. After the DMT group is removed, a nucleotide link is formed by binding with the activated 3′ phosphate group of another nucleotide injected from a solution. Since each nucleoside is injected to the column in solution state, the 5′-terminal at which binding with the monomer nucleoside (phosphoramidite) is protected with DMT. Accordingly, the oligo chain grows in 3′→5′ direction after the DMT group is removed.

1) Detritylation (removal of DMT): Trichloroacetic acid (TCA) is injected to separate 5′ DMT as cation, which is then removed by draining. The reaction occurs reversibly under anhydrous condition.

2) Coupling: Phosphoramidite is a chemically modified nucleoside. Coupling occurs through oxidation and reduction reactions of the following four compounds. Tetrazole (TET) and phosphoramidite react with the 5′ hydroxyl group of the support via the activated intermediate tetrazolyl phosphoramidite to form internucleotide phosphite.

\textcircled{1} diisopropylaminophosphoramidite

\textcircled{2} 3′-β-cyanoethyl protecting group

\textcircled{3} dimethoxytrityl protecting group protecting the 5′-hydroxyl group

\textcircled{4} benzoyl protecting group protecting the exocyclic amine of A and C; isobutyryl protecting group protecting the exocyclic amine of G (T lacks the exocyclic group)

3) Capping: Since about 2% of the 5′-hydroxyl group remains unreacted, the uncoupled group needs to be blocked from further chain elongation. This procedure is called capping and is achieved by acetylation.

4) Oxidation: Since the newly formed tricoordinated phosphite trimester is unstable, it is oxidized into a stable pentacoordinated phosphate triester.

5) Deprotection: After the synthesis is completed, DMT is removed by washing with acetonitrile and the support is separated from the column. In order to separate the synthesized DNA bound to the CPG, deprotection is performed for 8-15 hours at 55° C. using ammonium hydroxide.

6) Purification: The synthesized oligomer is a mixture of one having a desired sequence and capped one without being coupled with dNTP. Therefore, purification is required to extract only the desired oligomer. The purification may be performed by gel column purification, PAGE, H PLC, etc.

The above-described procedure will be explained in more detail.

The chemical synthesis of DNA involves a series of chemical reactions. Differently from the enzymatic synthesis occurring in vivo or in vitro by DNA polymerase, the synthesis occurs in 3′→5′ direction. What needs to be considered in the chemical synthesis of DNA is that there are many functional groups including the four nucleotides, phosphate, 5′ hydroxyl, etc. Accordingly, it is necessary to protect other functional groups during each stage of the synthesis.

1) Protection of Functional Groups

\textcircled{1} Amino Groups of Nucleotides

All the amino groups of DNA nucleotides should be protected. Otherwise, acetylation or phosphorylation may occur at the amino group during the synthesis. In general, a protecting group which is stable in acids and can be easily removed in alkalis is used. The amino groups of adenine (A) and cytosine (C) are protected with benzoyl, and the amino group of guanine (G) is protected with isobutyryl.

\textcircled{2} 5′ Hydroxyl Group

The 5′-OH should be protected during condensation, capping and oxidation reactions and be removed by weak acid (TCA) immediately before the nucleotide coupling. Dimethyltrityl (DMT) is used to protect the 5′-OH group.

\textcircled{3} Phosphate Group

Formerly, CH₃ was used to protect the phosphate group and removed at room temperature using thiophenol. Recently, the β-cyanoethyl protecting group which can be easily removed with concentrated ammonia water is used.

2) DNA Synthesis

DNA synthesis occurs in 3′→5′ direction. The 3′ hydroxyl group of the first nucleotide is attached to resin and a new nucleotide is added through four chemical reactions: detritylation (removal of DMT) at the 5′-terminal, addition of the new nucleotide (coupling), capping of the DNA chain where the addition did not occur, and oxidation of the phosphate group. After the reaction is completed, the protecting group is removed and the synthesized oligonucleotide is detached from the resin.

If the synthesis is carried out on the resin as described above, the several steps of reactions can be performed easily. Otherwise, purification is necessary after each step, which incurs great loss.

\textcircled{1} Detritylation (Removal of DMT)

In the first step of DNA synthesis, the DMT group protecting the 5′-OH group of the nucleoside derivative attached on the support is removed by treating with TCA. As a result, a free 5′-OH group that can react with phosphoramidite in the coupling step is obtained. This procedure is called detritylation. The DMT group released as byproduct is used to measure the synthesis efficiency such as coupling efficiency.

\textcircled{2} Coupling

Phosphoramidite is a nucleoside derivative. The diisopropylamine group at 3′-P provides stabilization. The compound is highly reactive with tetrazole. The β-cyanoethyl group protecting the 3′-P prevents side reactions and may be easily removed after synthesis by treating with concentrated ammonia. The 5′-OH group is protected with DMT. Except the phosphoramidite T, the amino group of the phosphoramidite C, A or G is bound to the benzoyl or isobutyryl group.

The reactant participating in the coupling needs to react quickly with the 5′-OH group, be synthesized easily and purified conveniently, and be a stable compound not reacting with H₂O or O₂. Prior to the coupling, the support should be thoroughly washed with acetonitrile to remove any substance having affinity for nucleosides. Then, the acetonitrile is dried out by refluxing argon gas. The phosphoramidite coupling reaction occurs via reagent containers 1, 2, 3, 4 and 5. As tetrazole is supplied to the column through a transfer tube, the resulting mixture exhibits weak acidity (pKa=4.8). The tetrazole transfers H⁺ to the nitrogen atom of the diisobutyryl group at 3′-P. Upon accepting H⁺, the amine has good affinity for nucleoside at the 5′-OH group. As a result, a tricoordinated phosphate group is formed at the intermediate nucleotide linkage and addition occurs.

\textcircled{3} Capping

The coupling reaction is not always quantitative and a small percentage (usually 0-2%) of the support-bound nucleotides may not participate in the addition reaction. The unreacted DNA chain needs to be capped by acetylating the free 5′-OH group so that it may not grow further. When equimolar acetic anhydride and N-methylimidazole (NMI) are transferred to the column at the same time, the strong acetylating agent reacts with the 5′-OH group.

\textcircled{4} Oxidation

The newly formed nucleotide linkage is a tricoordinated phosphite triester. Since the phosphite linkage is unstable, it can be cleaved easily upon reaction with acids. Accordingly, the tricoordinated phosphite trimester needs to be oxidized into a stable pentacoordinated phosphite trimester after the capping. Iodine acts as a weak oxidizing agent in a solution of oxygen-donating water and tetrahydrofuran (THF). When the iodine-water-lutidine-THF solution reaches the column, the tricoordinated phosphite is oxidized into pentacoordinated phosphite within 30 seconds. The iodine solution is removed with acetonitrile since it is detrimental in the next step. After this one cycle, one nucleotide is added.

After the synthesis is completed by repeating the above four steps according to the nucleotide sequence of the oligonucleotide to be synthesized, the DMT group remains attached to the 5′-terminal. Depending on the purification method of the synthesized DNA, the synthesis is terminated with the trityl group attached or removed. To summarize, the Y-shaped probe is synthesized in the order of 3′-E (right-side probe part)→D (right-side stem part)→C (linker)→B (left-side stem part)→A (left-side probe part)-5′.

\textcircled{5} Post-Synthetic Processing

The purification process following the synthesis is different depending on applications. After purification and drying, the oligonucleotide is stored in a small container. The synthesized oligonucleotide is quantitated before use. Then, it should be dissolved in DNase-free sterilized water (pH 7) or Tris-EDTA (TE, pH 7) buffer at proper concentration. In general, a concentration of 1 mg/mL is adequate.

At lower concentrations, the oligonucleotide is degraded easily. The amount of the oligonucleotide can be quantitated accurately and easily by measuring UV absorbance using a spectrophotometer.

1 OD unit=33 μg/mL single-stranded oligodeoxynucleotide (DNA)

1 mg DNA oligonucleotide=30 (OD)

1 μmol DNA oligonucleotide=10 (OD)

For example, if the absorbance of the synthesized oligonucleotide is OD260=3.3, the amount of the probe synthesized is 0.11 mg.

The above-described procedure can be performed automatically using a DNA synthesizer. Frequently used instruments include ABI's Applied Biosystems DNA synthesizer, BioLytic's Dr. Oligo-192 high throughput oligo synthesizer and Beckman's BeckMan Oligo 1000M. When parallel array synthesis is employed to reduce synthesis cost, 192 oligonucleotides can be synthesized at once from one instrument using a 96-well plate.

The Y-shaped probe designed in Example 1 may also be synthesized via a peptide nucleic acid (PNA) synthesis procedure. The resulting Y-shaped probe has the advantage of PNA. That is to say, the PNA/DNA dimer has a stronger binding ability than the DNA/DNA dimer because the electrically neutral PNA reduces repulsion with the target DNA. The strong binding provides the effect of increasing Tₘ by improving thermal stability of the PNA/DNA dimer. The Tₘ of the PNA/DNA dimer increased by about 1° C. per each nucleotide pair. Accordingly, when the number of PNA probes used in a chip is about 15, the Tₘ is increased by about 1° C. And, since the Tₘ value decreases greatly when a single nucleotide does not match, mutation of the nucleotide sequence can be detected easily. Further, the PNA is stable against nucleases or proteases. It is because the biological enzymes cannot recognize the amide backbone of the PNA. This biological stability can prevent the problems that may occur during DNA or RNA sample preparation and long-term storage. The PNA is also stable over wide ranges of pH and temperature since it is electrically neutral and has a strong covalent bonding. Whereas DNA is depurinated under acidic condition (pH 4.5-6.5), the PNA can be used for various applications since it is chemically stable under acidic and alkaline conditions.

### Examples 3-8

### Development of DNA Microarray for Diagnosis of HPV

The present disclosure provides a novel method for diagnosing human papilloma virus (HPV) using a DNA microarray on which the Y-shaped probe is spotted. In Examples 3-8, preparation of a Y-shaped probe for diagnosis of HPV (Example 3), fabrication of a DNA microarray by spotting the Y-shaped probe (Example 4), isolation and labeling of a DNA sample (Example 5), hybridization (Example 6), signal analysis (Example 7), and clinical diagnosis using the DNA microarray (Example 8) are described. Examples 3-8 describe an exemplary use of the Y-shaped probe and show that a DNA microarray using the Y-shaped probe is useful in diagnosis of important diseases.

The genome of HPV comprises a double-stranded DNA, in which E1-E7 early protein genes and L1 and L2 late protein genes are present. L1 and L2 encode the capsid protein protecting the genome by enclosing it. About 10% or more of the nucleotide sequence of the L1 gene varies depending on the type of HPV. Hence, the genotype of HPV can be identified by detecting the difference. HPV infiltrates into the epithelium of human skin or mucous membranes, thereby causing inflammation, hyperproliferation, and even cancer (National Network of STD/HIV Prevention Training Center. Genital human papillomavirus infection. February 2008).

There are about 120 types of HPV depending on the genotype. About 40 types of them are the so-called anogenital type HPV affecting the anogenital regions, i.e. the skin and mucous membranes of the vagina, cervix, urethra and penis. While the majority of HPV infection cause no symptoms, some types can cause warts. Others can lead to precancerous lesions such as high-grade squamous intraepithelial lesion (SIL) or cervical intraepithelial neoplasia, and some of them develop into cancer. The HPV types causing precancerous lesions and cancers are called high-risk type HPV, and those that do not are called low-risk type HPV. The high-risk type HPV includes HPV types 16, 18, 31, 33, 35, 39, 45, 51, 52, 56, 58, 59, 68 and 82. And, the low-risk type HPV includes HPV types 6, 11, 34, 40, 42, 43, 44, 54, 55, 61, 62, 72 and 81. Probable high-risk types include HPV types 26, 53, 66, 67, 69, 70 and 73. Besides, there are other types that are not definitely classified, which are HPV types 7, 10, 27, 30, 32, 57, 83, 84 and 91. For the high-risk type HPV, the E6 and E7 genes are the oncogenes associated with cancer. They inactivate the p53 and retinoblastoma (Rb) genes which are the most important tumor suppressor genes in human and thus trigger carcinogenesis. More than 99% of cervical cancer is caused by the high-risk type HPV, and the HPV gene fragment such as E6/E7 is found in nearly all cancer cells (Munoz N, Bosch F X, de Sanjose S, Herrero R, Castellsague X, Shah K V, Snijders P J, Meijer C J and International Agency for Research on Cancer Multicenter Cervical Cancer Study Group. Epidemiologic classification of human papillomavirus types associated with cervical cancer. New England Journal of Medicine. 2003; 348: 518-527).

HPV infection is hardly detected by culturing, staining, histological inspection or immunological inspection and can only be accurately diagnosed by genetic testing. There are three kinds of HPV genetic testing. The first is to simply investigate the presence of HPV. A representative example is amplification of the consensus sequence, i.e. invariant nucleotide sequence, of the HPV gene by PCR followed by identification through, for example, electrophoresis. The second is the so-called genotyping analysis of identifying not only the presence of HPV but also its type. The gold standard test is to performing PCR and analyzing the genotype by automated nucleotide sequencing of the product. However, since this method requires a lot of cost, time and labor, it is being replaced by the HPV DNA microarray. A plurality of probes specific for HPV types are spotted on a solid support and a PCR product of the sample DNA is placed thereon and hybridized. Then, the result is analyzed using a scanner. The third is intermediary of the two test methods. The hybrid capture assay (Digene Corporation, Gaithersburg, Md., USA) is an example. Although it allows to identify of whether HPV exists and whether the HPV is high-risk type or low-risk type, accurate genotyping is impossible. In addition, only 13 high-risk type HPVs and 7 low-risk type HPVs can be identified, and other 20 or more HPV types cannot be identified (Kim K H, Yoon M S, Na Y J, Park C S, Oh M R, Moon W C. Development and evaluation of a highly sensitive human papillomavirus genotyping DNA chip. Gynecol Oncol. 2006; 100(1): 38-43; Selva L, Gonzalez-Bosquet E, Rodriguez-Plata^{a} M T, Esteva C, Sunol M and Munoz-Almagro C. Detection of human papillomavirus infection in women attending a colposcopy clinic. Diagnostic Microbiology and Infectious Disease. 2009; 64: 416-421).

Genetic testing of HPV is very important not only medically but also socioeconomically. The reason includes the followings.

Firstly, HPV infection is the most common sexually transmitted infection in human with the highest prevalence rate. In the US, HPV infection is found in 26.8% of women aged between 14 and 59 and it is thought that 80% of women are infected at least once. The infection occurs well particularly in sexually active, fertile women, and the prevalence is estimated to increase. Hence, the HPV market is very large and the HPV testing is of great economic value (U.S. Department of Health And Human Services, Centers for Disease Control and Prevention, National Center for HIV/AIDS, Viral Hepatitis, STD, and TB, Prevention Division of STD Prevention. Sexually Transmitted Disease Surveillance 2008. Division of STD Prevention. 2009: November; Tchernev G. Sexually transmitted papillomavirus infections: epidemiology pathogenesis, clinic, morphology, important differential diagnostic aspects, current diagnostic and treatment options. An Bras Dermatol. 2009; 84(4): 377-89).

Secondly, HPV is clearly proven to cause cancer in human. HPV, particularly the high-risk type HPV, is the cause of nearly all cases of cervical cancer. Globally, about 500,00 women are affected by cervical cancer and more than 270,000 are killed every year. Moreover, most cases of anal cancer, oral cancer, pharyngeal cancer and laryngeal cancer are associated directly or indirectly with HPV. HPV testing is of great importance in that HPV can be fatal by causing cancer. Also, cancers and precancerous lesions of the cervix, anus, etc. can be diagnosed early by HPV testing. Indeed, it is shown that HPV testing is superior in prediction sensitivity of cervical cancer than the Papanicolaou test, or Pap smear, which is the standard screening method for early diagnosis of cervical cancer. Accordingly, it is approved as the cervical cancer screening test in several countries including the US (Parkin M, F. Bray F, J. Ferlay J and P. Pisani P. Global cancer statistics, 2002. C.A. Cancer J. Clin. 2005; National Network of STD/HIV Prevention Training Center. Genital human papillomavirus infection. February 2008).

Thirdly, the recently developed HPV vaccine is the first case of preventing viral infection and cancer through vaccination. Two types of HPV vaccines are currently available. Gardasil (Merck & Co. Inc., Whitehouse Station, N.J., USA) is a quadrivalent vaccine prepared against HPV types 16, 18, 6 and 11. The other, Cervarix (GlaxoSmithKline Biologicals, Rixensart, Belgium), is a bivalent vaccine designed to prevent infection from HPV types 16 and 18. These vaccines are the most effective for adolescent girls before sexual activity, and the efficacy decreases in women who have been infected by HPV16 or HPV18 before. For this reason, vaccination to adult women are controversial. But, vaccination may be possible unless the HPV infection is by type 16 or 18. Accordingly, it is becoming more and more important to identify not just the HPV infection but the accurate type of HPV (Selva L, Gonzalez-Bosquet E, Rodriguez-Plata^{a} M T, Esteva C, Sunol M and Munoz-Almagro C. Detection of human papillomavirus infection in women attending a colposcopy clinic. Diagnostic Microbiology and Infectious Disease. 2009; 64: 416-421; Reynales-Shigematsu L M, Rodrigues E R, Lazcano-Ponce E. Cost-effectiveness analysis of a quadrivalent human papilloma virus vaccine in Mexico. Arch Med Res. 2009 August; 40(6): 503-13).

As described in the above literature, it is keenly needed to test the presence and genotype of HPV accurately and quickly, at low cost and in large scale. The DNA microarray is a promising tool for such test.

Several HPV DNA microarray products are commercially available. Representative examples include the HPV DNA chip test (MyGene Co. and Biomedlab Co., Seoul, Korea), GG HPV DNA chip (Goodgene Inc., Seoul, Korea) and Clinical Arrays Papillomavirus Humano (CAPH) chip (Genomica S.A.U., Madrid, Spain). These products are similar in that they target the consensus sequence of the HPV L1-E6/E7 genes and HPV oligonucleotide probes specific for 22-44 anogenital type HPVs are spotted on the glass slide. Among them, the GG HPV chip and the CAPH chip are advantageous in that a probe for the human beta-globin gene as internal reference gene is spotted together. However, the existing microarrays are not considered perfect with regard to the problems described above, i.e. noise processing and normalization, signal analysis, statistical analysis, quality control, etc. For example, when a weak positive signal occurs at a spot for a specific type of HPV or when, although a strong signal occurs, the background signal is also strong, it is difficult to identify whether the signal is true positive or false positive. Also, it is not easy to identify the false negative error, and there remain unsolved problems such as reproducibility, quality control, etc.

In the HPV DNA microarray of the present disclosure, a Y-shaped probe is used to solve the aforesaid problems of the existing HPV DNA chip. The Y-shaped probe comprises a probe specific for HPV subtype in the L1 gene on one side and a probe for human beta-globin as an internal reference or control gene on the other side. Then, after fluorescence-labeling the HPV L1 and human beta-globin genes differently with, for example, Cy-5 and Cy-3, and performing PCR, the product is hybridized on the microarray and the result is analyzed using a fluorescence scanner. The ratio of the normalized Cy-5 signal to the Cy-3 signal is analyzed for each spot after excluding the background signal to determine whether the signal is true positive. In this manner, false positive and false negative errors can be minimized. In addition, errors occurring from different spots can be reduced and signal reading, statistical analysis and quality control can be accomplished more appropriately. A HPV DNA microarray product of the same type as that of the present disclosure has never been reported.

The HPV DNA microarray of the present disclosure is expected to be very helpful not only in diagnosis of HPV itself but also in screening, early detection, prevention and treatment of cervical and other cancers caused by HPV. Specifically, it may provide the optimum test for early diagnosis of cervical cancer, anal cancer, oral cancer, etc. caused by HPV and may be helpful in investigating the applicability of HPV vaccines. In addition, it may be helpful when designing a DNA vaccine or a dendritic cell vaccine customized for the specific type of HPV found in a cancer patient. These vaccines may trigger cell-mediated immunity against HPV unlike the preventive vaccines, thereby inducing T cells to kill not only HPV but the abnormal cells infected by HPV and thus providing anticancer effect (Monie A, Tsen S W, Hung C F, Wu T C. Therapeutic HPV DNA vaccines. Expert Rev Vaccines. 2009; 8(9): 1221-35).

The HPV DNA microarray product of the present disclosure includes not only the microarray but also instructions about the PCR reagent, hybridization reagent, sampling kit and scanner.

### Example 3

### Preparation of Y-Shaped Probe for HPV

The Y-shaped probe and PCR primer for HPV genotyping were designed.

First, from the HPV genome, a portion which is a consensus sequence and the HPV type is identifiable with at least three nucleotide sequences differing for the different types was selected. It corresponds to the 1024th to 1205th sequences of the reference nucleotide sequence of the HPV L1 gene. A primer was designed to amplify them by PCR. The most appropriate regions for the HPV types were selected from the PCR product and the right-side probe part of the Y-shaped probe was designed to be complementary thereto. In a similar manner, a primer was designed for PCR of the human beta-globin (HBB) gene as internal reference gene. The most appropriate regions were selected from the PCR product and the left-side probe part of the Y-shaped probe was designed to be complementary thereto.

3.1. Designing of PCR Primer for HPV

The DNA chip kit of the present disclosure comprises primers for amplifying HPV types selected from SEQ ID NOS 1-4 and a human beta-globin primer. The combinations of the oligonucleotide primers required for PCR of the HPV L1 gene to be tested and the human beta-globin gene are summarized in Table 1.

TABLE 1
Oligonucleotide primers for PCR

| No | Gene | Name | Sequence (5′->3′) | Mer | TM (° C.) | GC % |
|---|---|---|---|---|---|---|
| SEQ ID | HBB | H1Cy-3 | Cy-3-ACACAACTGTGTTCACTAGC | 20 | 46.8 | 45 |
| NO 1 | | | | | | |
| SEQ ID | HBB | H2 | CAAACTTCATCCACGTTCACC | 21 | 56.6 | 48 |
| NO 2 | | | | | | |
| SEQ ID | HPV | L1F | GCMCAGGGWCATAAYAATGG | 20 | 66 | 50 |
| NO 3 | L1 | | | | | |
| SEQ ID | HPV | L1R | Cy-5-AATAAACTGTAAATCATATTCCTC | 24 | 47 | 25 |
| NO 4 | L1 | | | | | |

(IUPAC code meaning; M: A or C, W: A or T, Y: C or T)

The primers are labeled with various labels, and various known labels may be used. For example, Cy-5, Bodipsy, Cy-3, Alexa 532, Alexa 546, Rodamin, TAMRA, FAM, FITC, Fluor X, Alexa 488, Alexa 568, ROX, Texas Red, Alexa 594, etc. may be used.

3.2. Designing of Y-Shaped Probe for HPV

A Y-shaped probe was designed for HPV genotyping according to the design principle described in Example 1.

3.2.1. Left-Side and Right-Side Probe Parts (A and E in FIG. 1)

A probe for the human beta-globin gene (CGG CAG ACT TCT CCT C) was arranged in reverse direction on the left-side probe part (A in FIG. 1) of the Y-shaped probe, and the sequence of the HPV L1 gene was arranged in forward direction on the right-side probe part (E in FIG. 1). The design was varied depending on the HPV types.

3.2.2. Stem Parts (B and D in FIG. 1)

The sequence CCCTAA which is reverse to the human telomere sequence was arranged on the left-side stem part (B in FIG. 1), and the sequence TTAGGG complementary thereto was arranged on the (D in FIG. 1).

3.2.3. Linker Part (X in FIG. 1)

The linker was designed using an internal amino modifier C6 dT (iAmMC6T). Y-shaped probes were designed and synthesized for all the 44 HPV types known to invade the cervix according to the method described in Example 2. The name, SEQ ID NO and genotype of the Y-shaped probes for HPV are summarized in Table 2.

However, the Y-shaped probe may be varied as desired depending on purposes and applications. The sequence for the L1 gene of each type of HPV may be arranged on the right-side probe part and the left-side probe part may be changed variously. For example, a sequence universal for all the HPV types, such as the L1 or L2 gene, may be arranged on the left-side probe.

Also, a sequence specific for the HPV L2 gene of each HPV type may be arranged on the left-side probe part for dual testing. In this case, the sequence should be of the same HPV type as that of the right-side probe part. Also, a sequence specific for the E6/E7 gene of each HPV type may be arranged on the left-side probe part for dual testing. In this case, the sequence should be of the same HPV type as that of the right-side probe part.

TABLE 2
Sequence of Y-shaped probe for HPV DNA chip

| No | Name | Sequence (5′->3′) | Mer | TM (° C.) | GC % |
|---|---|---|---|---|---|
| SEQ ID NO 5 | HPV6YP | CGGCAGACTTCTCCTCCCCTAA-iAmMC6T-TTAGGG | 52 | 55.6 | 45 |
| | | GCATCCGTAACTACATCTTCCA | | | |
| SEQ ID NO 6 | HPV7YP | CGGCAGACTTCTCCTCCCCTAA-iAmMC6T-TTAGGG | 52 | 51.6 | 36 |
| | | ACACCAACACCATATGACAATA | | | |
| SEQ ID NO 7 | HPV10YP | CGGCAGACTTCTCCTCCCCTAA-iAmMC6T-TTAGGG | 48 | 60.2 | 72 |
| | | GCCTCCCCTGCCACTACG | | | |
| SEQ ID NO 8 | HPV11YP | CGGCAGACTTCTCCTCCCCTAA-iAmMC6T-TTAGGG | 48 | 54.4 | 50 |
| | | ATTTGCTGGGGAAACCAC | | | |
| SEQ ID NO 9 | HPV16YP | CGGCAGACTTCTCCTCCCCTAA-iAmMC6T-TTAGGG | 52 | 49.9 | 36 |
| | | TGCCATATCTACTTCAGAAACT | | | |
| SEQ ID NO 10 | HPV18YP | CGGCAGACTTCTCCTCCCCTAA-iAmMC6T-TTAGGG | 51 | 51.5 | 52 |
| | | TCTACACAGTCTCCGTACCTG | | | |
| SEQ ID NO 11 | HPV26YP | CGGCAGACTTCTCCTCCCCTAA-iAmMC6T-TTAGGG | 52 | 57.9 | 45 |
| | | ATTATCTGCAGCATCTGCATCC | | | |
| SEQ ID NO 12 | HPV27YP | CGGCAGACTTCTCCTCCCCTAA-iAmMC6T-TTAGGG | 56 | 54.2 | 38 |
| | | CAGCTGAGGTGTCTGATAATACTAAT | | | |
| SEQ ID NO 13 | HPV30YP | CGGCAGACTTCTCCTCCCCTAA-iAmMC6T-TTAGGG | 50 | 52.6 | 40 |
| | | AACCACACAAACGTTATCCA | | | |
| SEQ ID NO 14 | HPV31YP | CGGCAGACTTCTCCTCCCCTAA-iAmMC6T-TTAGGG | 52 | 54.7 | 41 |
| | | CTGCAATTGCAAACAGTGATAC | | | |
| SEQ ID NO 15 | HPV32YP | CGGCAGACTTCTCCTCCCCTAA-iAmMC6T-TTAGGG | 55 | 46.4 | 32 |
| | | GACACATACAAGTCTACTAACTTTA | | | |
| SEQ ID NO 16 | HPV33YP | CGGCAGACTTCTCCTCCCCTAA-iAmMC6T-TTAGGG | 55 | 51.7 | 44 |
| | | GCACACAAGTAACTAGTGACAGTAC | | | |
| SEQ ID NO 17 | HPV34YP | CGGCAGACTTCTCCTCCCCTAA-iAmMC6T-TTAGGG | 49 | 48 | 52.6 |
| | | CCACAAGTACAACTGCACC | | | |
| SEQ ID NO 18 | HPV35YP | CGGCAGACTTCTCCTCCCCTAA-iAmMC6T-TTAGGG | 55 | 52.6 | 44 |
| | | TCTGCTGTGTCTTCTAGTGACAGTA | | | |
| SEQ ID NO 19 | HPV39YP | CGGCAGACTTCTCCTCCCCTAA-iAmMC6T-TTAGGG | 59 | 55.7 | 41 |
| | | ACCTCTATAGAGTCTTCCATACCTTCTAC | | | |
| SEQ ID NO 20 | HPV40YP | CGGCAGACTTCTCCTCCCCTAA-iAmMC6T-TTAGGG | 46 | 50 | 63 |
| | | AGTCCCCCACACCAAC | | | |
| SEQ ID NO 21 | HPV42YP | CGGCAGACTTCTCCTCCCCTAA-iAmMC6T-TTAGGG | 48 | 50.1 | 50 |
| | | CACTGCAACATCTGGTGA | | | |
| SEQ ID NO 22 | HPV43YP | CGGCAGACTTCTCCTCCCCTAA-iAmMC6T-TTAGGG | 52 | 54.7 | 45.4 |
| | | GCCCAGTACATATGACAATGCA | | | |
| SEQ ID NO 23 | HPV44YP | CGGCAGACTTCTCCTCCCCTAA-iAmMC6T-TTAGGG | 48 | 49.7 | 61.1 |
| | | TACACAGTCCCCTCCGTC | | | |
| SEQ ID NO 24 | HPV45YP | CGGCAGACTTCTCCTCCCCTAA-iAmMC6T-TTAGGG | 49 | 53.7 | 47 |
| | | CACAAAATCCTGTGCCAAG | | | |
| SEQ ID NO 25 | HPV51YP | CGGCAGACTTCTCCTCCCCTAA-iAmMC6T-TTAGGG | 50 | 52.3 | 45 |
| | | GGTTTCCCCAACATTTACTC | | | |
| SEQ ID NO 26 | HPV52YP | CGGCAGACTTCTCCTCCCCTAA-iAmMC6T-TTAGGG | 52 | 56.6 | 41 |
| | | GCTGAGGTTAAAAAGGAAAGCA | | | |
| SEQ ID NO 27 | HPV53YP | CGGCAGACTTCTCCTCCCCTAA-iAmMC6T-TTAGGG | 53 | 56.6 | 48 |
| | | CGCAACCACACAGTCTATGTCTA | | | |
| SEQ ID NO 28 | HPV54YP | CGGCAGACTTCTCCTCCCCTAA-iAmMC6T-TTAGGG | 47 | 53.3 | 59 |
| | | TACAGCATCCACGCAGG | | | |
| SEQ ID NO 29 | HPV55YP | CGGCAGACTTCTCCTCCCCTAA-iAmMC6T-TTAGGG | 54 | 51.9 | 42 |
| | | CTACAACTCAGTCTCCATCTACAA | | | |
| SEQ ID NO 30 | HPV56YP | CGGCAGACTTCTCCTCCCCTAA-iAmMC6T-TTAGGG | 64 | 55.1 | 32.4 |
| | | GACTATTAGTACTGCTACAGAACAGTTAAGTAAA | | | |
| SEQ ID NO 31 | HPV57YP | CGGCAGACTTCTCCTCCCCTAA-iAmMC6T-TTAGGG | 54 | 53.3 | 38 |
| | | CCACTGTAACCACAGAAACTAATT | | | |
| SEQ ID NO 32 | HPV58YP | CGGCAGACTTCTCCTCCCCTAA-iAmMC6T-TTAGGG | 53 | 54.4 | 43 |
| | | TGCACTGAAGTAACTAAGGAAGG | | | |
| SEQ ID NO 33 | HPV59YP | CGGCAGACTTCTCCTCCCCTAA-iAmMC6T-TTAGGG | 59 | 56.5 | 38 |
| | | TCTATTCCTAATGTATACACACCTACCAG | | | |
| SEQ ID NO 34 | HPV61YP | CGGCAGACTTCTCCTCCCCTAA-iAmMC6T-TTAGGG | 50 | 57.8 | 55 |
| | | TGCTACATCCCCCCCTGTAT | | | |
| SEQ ID NO 35 | HPV62YP | CGGCAGACTTCTCCTCCCCTAA-iAmMC6T-TTAGGG | 50 | 53 | 50 |
| | | ACTATTTGTACCGCCTCCAC | | | |
| SEQ ID NO 36 | HPV66YP | CGGCAGACTTCTCCTCCCCTAA-iAmMC6T-TTAGGG | 56 | 56.9 | 31 |
| | | AATGCAGCTAAAAGCACATTAACTAA | | | |
| SEQ ID NO 37 | HPV67YP | CGGCAGACTTCTCCTCCCCTAA-iAmMC6T-TTAGGG | 53 | 51.8 | 30 |
| | | AAAATCAGAGGCTACATACAAAA | | | |
| SEQ ID NO 38 | HPV68bYP | CGGCAGACTTCTCCTCCCCTAA-iAmMC6T-TTAGGG | 61 | 54.9 | 35.5 |
| | | CTACTACTACTGAATCAGCTGTACCAAATAT | | | |
| SEQ ID NO 39 | HPV68aYP | CGGCAGACTTCTCCTCCCCTAA-iAmMC6T-TTAGGG | 53 | 53.2 | 52 |
| | | CAGACTCTACTGTACCAGCTGTG | | | |
| SEQ ID NO 40 | HPV69YP | CGGCAGACTTCTCCTCCCCTAA-iAmMC6T-TTAGGG | 55 | 61 | 40 |
| | | CACAATCTGCATCTGCCACTTTTAA | | | |
| SEQ ID NO 41 | HPV70YP | CGGCAGACTTCTCCTCCCCTAA-iAmMC6T-TTAGGG | 47 | 55.5 | 59 |
| | | CCGAAACGGCCATACCT | | | |
| SEQ ID NO 42 | HPV72YP | CGGCAGACTTCTCCTCCCCTAA-iAmMC6T-TTAGGG | 51 | 55.1 | 52 |
| | | CACAGCGTCCTCTGTATCAGA | | | |
| SEQ ID NO 43 | HPV73YP | CGGCAGACTTCTCCTCCCCTAA-iAmMC6T-TTAGGG | 57 | 54.4 | 48 |
| | | AGGTACACAGGCTAGTAGCTCTACTAC | | | |
| SEQ ID NO 44 | HPV81YP | CGGCAGACTTCTCCTCCCCTAA-iAmMC6T-TTAGGG | 50 | 56.5 | 55 |
| | | GCTACATCTGCTGCTGCAGA | | | |
| SEQ ID NO 45 | HPV82YP | CGGCAGACTTCTCCTCCCCTAA-iAmMC6T-TTAGGG | 49 | 50.5 | 42 |
| | | CTCCAGCAAACTTTAAGCA | | | |
| SEQ ID NO 46 | HPV83YP | CGGCAGACTTCTCCTCCCCTAA-iAmMC6T-TTAGGG | 57 | 55.9 | 48 |
| | | TGCTGCTACACAGGCTAATGA | | | |
| SEQ ID NO 47 | HPV84YP | CGGCAGACTTCTCCTCCCCTAA-iAmMC6T-TTAGGG | 54 | 57.7 | 33 |
| | | ACCGAATCAGAATATAAACCTACCAAT | | | |
| SEQ ID NO 48 | HPV90YP | CGGCAGACTTCTCCTCCCCTAA-iAmMC6T-TTAGGG | 53 | 55.7 | 43 |
| | | ACAAACACCCTCTGACACATACA | | | |
| SEQ ID NO 49 | HPV91YP | CGGCAGACTTCTCCTCCCCTAA-iAmMC6T-TTAGGG | 58 | 57.3 | 39 |
| | | TCTGTGCTACCTACTACATATGACAACA | | | |
| SEQ ID NO 50 | HPVUYP | CGGCAGACTTCTCCTCCCCTAA-iAmMC6T-TTAGGG | 60 | 61.2 | 34 |
| | | TTGTTGGGDTAATCAGTTGTTTGTTACTGT | | | |

### Example 4

### Fabrication of DNA Microarray (Chip) Using Y-Shaped HPV Probe

The Y-shaped probe prepared using the nucleotide sequence of Table 2 according to the method of Example 2 was mixed with a suitable reagent and spotted on a glass slide for a microscope using an arrayer to fabricate a DNA microarray or DNA chip for HPV genotyping. Details are as follows.

4.1. Spotting of Probes for HPV L1 Gene and Human Beta-Globin Gene on DNA Chip

Grids were formed to easily identify the HPV type based on the fluorescence signal occurring after the hybridization on the chip.

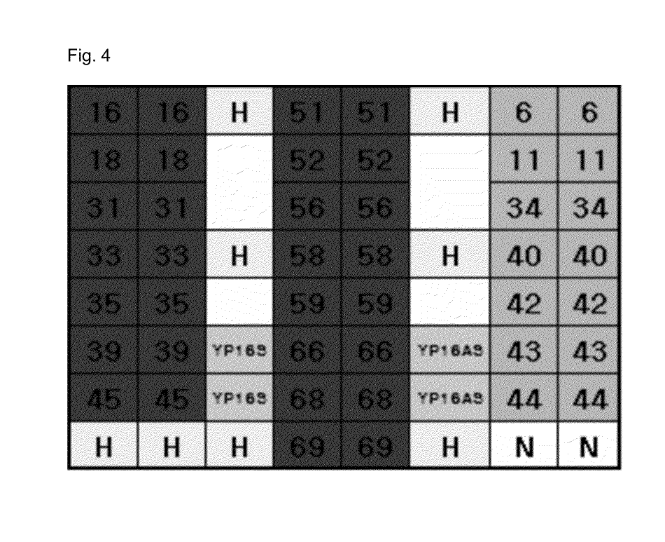

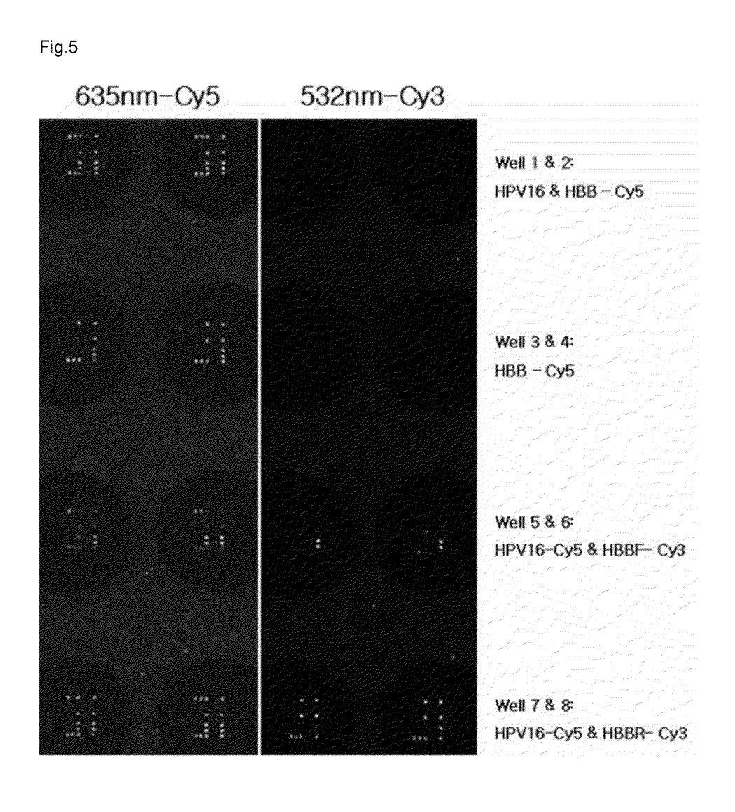

The order of the probes and the arrangement of the grids are schematically shown in FIG. 4. FIG. 4 also shows the spotting order and position of the DNA probe capable of detecting only the 22 L1 genes which are the most important of the HPV types among those described in Table 2. FIG. 5 shows the HPV DNA chip fabricated according to the present disclosure. 8 wells are present on one slide and the probe having the grids shown in FIG. 4 is spotted on each well. 8 different samples were placed on the wells so that they could be tested simultaneously.

Each Y-shaped probe was spotted using an arrayer. The same probes were spotted in duplicate so that each HPV type could be tested at least 2 times and up to 4 times.

4.2. Preparation of Oligonucleotide Probe Solution to be Spotted on Chip and Division into Master Plate

The Y-shaped probe synthesized by attaching amine to the internal C6dT moiety as described in Example 3 was purified by high-performance liquid chromatography (HPLC) and dissolved in sterilized triply distilled water to a final concentration of 200 pM. Thus prepared probes were mixed with a microspotting solution at 1:4.3 to a final concentration of 38 pM. The resulting mixture was sequentially divided in to a 384-well master plate.

4.3. Spotting and Fixation of Probes

Using Q arrayer2 (Genetixs, UK) or an arrayer comparable thereto, the spotting solution containing the probes was transferred from the master plate and spotted on an aldehyde-coated glass slide in duplicate (double hit). As for the glass slide, Luminano aldehyde LSAL-A, silicon wafer or a product comparable thereto may be used. Each spot can be spotted with a size of about 10-200 μm. The DNA chip prepared by spotting the probes on the glass slide was reacted at room temperature for 15 minutes inside a glass jar maintained at 80% humidity and then post-treated according to a known method (Zammatteo, N., L. Jeanmart, S. Hamels, S. Courtois, P. Louette, L. Hevesi, and J. Remacle. 2000. Comparison between different strategies of covalent attachment of DNA to glass surfaces to build DNA microarrays. Anal. Biochem. 280: 143-150.).

4.4. Washing and Storing of Microarray

After the post-treatment, the fixated slide was baked in a drying oven for 1.5 hours at 120° C. Then, the slide was washed in 0.2% sodium dodecylsulfate (SDS) solution twice for 2 minutes, and transferred to triply distilled water, then washed twice for 2 minutes. Thereafter, the slide was dipped in triply distilled water heated to 95° C. for 3 minutes, whereby the oligonucleotide probes attached on the slide were denatured, and then washed in triply distilled water for 1 minute. After washing, the slide was reduced for 15 minutes in a blocking solution (1 g NaBH₄, 300 mL PBS, 100 mL ethanol), washed twice in 0.2% SDS solution for 2 minutes, and then transferred to triply distilled water and washed twice for 2 minutes. Water remaining on the slide was removed by centrifuging at 800 rpm for 1.5 minutes, and then the slide was put in a slide box and stored in a desiccator at room temperature.

The reaction described in Example 5 was performed using thus prepared chip of the present disclosure.

### Example 5

### Preparation of Sample

The HPV L1 gene separated from the DNA of the sample and the positive control gene human beta-globin gene were labeled with fluorescent dyes by PCR as follows.

5.1. Isolation of DNA from Sample

DNA was separated from the control substance and the clinical sample. As positive control substance, Caski cervical cancer cells containing the cDNA of HPV 16 were purchased from the American Type Culture Collection (ATCC). Human cervical tissue, cervical swab, and cervical and vaginal washing samples were obtained and total DNA was isolated from each sample using the QiaAmp DNA Mini Kit (Qiagene).

5.2. PCR

The primers for PCR of HPV include the primer for PCR selected from a group consisting of SEQ ID NOS 1-4 and the human beta-globin primer. PCR was performed as follows.

PCR for detection of HPV infection was performed using 15 μL of SuperTaq plus pre-mix (10× buffer 2.5 μL, 10 mM MgCl₂ 3.75 μL, 10 mM dNTP 0.5 μL, Taq polymerase 0.5 μL) purchased from Super Bio (Seoul, Korea). As described in Table 1, 1 μL (10 pmol/μL) of each of L1F, L1R, H1 and H2 primers was added thereto and then 4.0 μL (150 ng/μL) of sample template DNA was added. Then, distilled water was added to make the total volume of the reaction solution 30 μL.

For PCR of the human beta-globin gene, the reaction solution containing the primer was subjected to predenaturation at 95° C. for 5 minutes followed by 40 cycles of 30 seconds at 95° C., 30 seconds at 50° C. and 30 seconds at 72° C. Then, extension was performed at 72° C. for 5 minutes. The reaction solution containing the HPV H1 and H2 primers was subjected to predenaturation at 95° C. for 5 minutes followed by 40 cycles of 30 seconds at 95° C., 30 seconds at 50° C. and 30 seconds at 72° C. Then, extension was performed at 72° C. for 5 minutes.

5.3. Confirmation of PCR Result

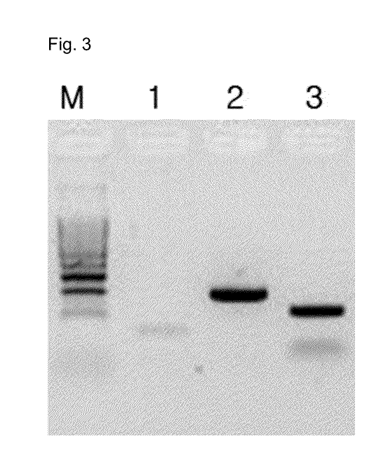

PCR was performed after labeling the HPV L1 gene with Cy-5 and the HBB gene with Cy-3. The product was subjected to electrophoresis on 0.8% agarose gel. FIG. 3 shows the result of amplifying the HPV L1 gene and the human beta-globin gene by PCR and performing electrophoresis.

### Example 6

### Hybridization

Hybridization was performed on the microarray as follows.

6.1. Hybridization

10 μL of the PCR product of each sample DNA was mixed to a final volume of 50 μL on the slide chip on which the Y-shaped probe was spotted. After denaturation at 95° C. for 5 minutes, the slide chip was kept on ice for 3 minutes. Then, after adjusting the final volume to 100 μL by adding 50 μL of hybridization solution, reaction with the probe fixed on the slide was carried out for 30 minutes at 45° C. The hybridization solution was prepared by mixing 2 mL of 20×SSC, 1.7 mL of 90% glycerol and 6.3 mL of 50 mM phosphate buffer.

6.2. Washing

Immediately after the hybridization was completed, the well cover was removed from the DNA chip and the chip was immersed in 3×SSPE solution (NaCl (26.295 g), NaH₂PO₄-1H₂O (4.14 g), Na₂EDTA (1.11 g) dissolved in 1 L of distilled water; adjusted to pH 7.4 with 10 N NaOH). After washing at room temperature for 2 minutes and then again with 1×SSPE solution (NaCl (8.765 g), NaH₂PO₄-1H₂O (1.38 g), Na₂EDTA (0.37 g) dissolved in 1 L of distilled water; adjusted to pH 7.4 with 10 N NaOH) at room temperature for 2 minutes, centrifugation was performed at 800 rpm at room temperature for 1.5 minutes followed by drying.

### Example 7

### Confirmation of Result after Hybridization

After removal of nonspecific signals as much as possible through washing, the dried slide was subjected to analysis of fluorescence signals and images using a fluorescence scanner. As for the scanner, a dual-color scanner such as the GenePix 4000B scanner (Axon, USA), ScanArray Lite (Packard Bioscience, USA) or an equipment comparable thereto may be used.

When the GenePix Pro 6.0 program is used, the scanning result is interpreted as follows. The HPV chip grids are fixed on the images scanned at 635 nm and 532 nm and “Align Features in All Blocks” is executed. Then, after executing “Analyze”, “Results” are saved as a grs file. The saved grs file is read using the Excel program and to compute the signal-to-background gray level ratio (SBR; the ratio of the pixel value of the image of each spot to that of the background) using the equation: HPV type SBR=F532 Median÷B532 Median)/(HBB SBR=F635 Median÷B635 Median). The SBR value for each HPV genotype is computed at least two spots. The result is regarded true positive only when the SBR value for the control gene HBB is at least 2.5 and the value of the SBR of HPVL1 divided by the SBR of HBB is 1 or greater. But, this cut-off level and criterion of evaluation can vary depending on the microarray type.

FIG. 5 shows scan images obtained from a cervical sample infected with HPV type 16. FIG. 5 shows a result of spotting the Y-shaped probe of the present disclosure simultaneously on 22 HPV chips fabricated using the grids of FIG. 4 and hybridizing with HPV-16 (labeled with Cy5) and HBB (labeled with Cy3). That is to say, the left image is a result of scanning at 635 nm where Cy-5 is detectable and the right image is a result of scanning at 532 nm where Cy-3 is detectable. In FIG. 5, wells 1 and 2 contain the samples labeled with HPV 16-Cy5 and HBB-Cy5, respectively. Wells 3 and 4 contain the samples labeled with HBB-Cy5. Wells 5 and 6 contain the samples labeled with HPV 16-Cy5 and HBB forward primer-Cy3, respectively. And, wells 7 and 8 contain the samples labeled with HPV 16-Cy5 and HBB reverse primer-Cy3, respectively.

As seen from FIG. 5, since the HBB gene included in the portion A of the Y-shaped probe is in antisense direction, the primer binding thereto binds with the PCR product in which Cy-3 is incorporated to the forward primer. And, since the HPV L1 gene included in the portion A is in sense direction, the primer binding thereto binds with the PCR product in which Cy-5 is incorporated to the reverse primer.

That is to say, it was confirmed that, in wells 1 and 2, for the PCR product in which both HPV 16 and HBB are labeled with Cy-5, the spots are detected at 635 nm where only Cy-5 is detectable and are not detected at 532 nm where only Cy-3 is detectable.

In wells 3 and 4, for the PCR product in which only HBB is labeled with Cy-5, the spots are detected at 635 nm where only Cy-5 is detectable and are not detected at 532 nm where only Cy-3 is detectable.

In wells 5 and 6, for the PCR product in which HPV 16 is labeled with Cy-5 and the forward primer of HBB is labeled with Cy-3, only the HPV 16 and YP16AS spots are detected at 635 nm where only Cy-5 is detectable and only the YP16AS spots are detected at 532 nm where only Cy-3 is detectable.

In wells 7 and 8, for the PCR product in which HPV 16 is labeled with Cy-5 and the reverse primer of HBB is labeled with Cy-3, the all the HPV 16, YP16S and YP16AS spots are detected at 635 nm where only Cy-5 is detectable but only the HBB spots are not detected at 532 nm where only Cy-3 is detectable.

Thus, it was confirmed that, for the PCR product in which HPV 16 is labeled with Cy-5 and the forward primer of HBB is labeled with Cy-3, only the HPV 16 and YP16AS spots are detected at 635 nm where only Cy-5 is detectable and only the YP16AS spots are detected at 532 nm where only Cy-3 is detectable.

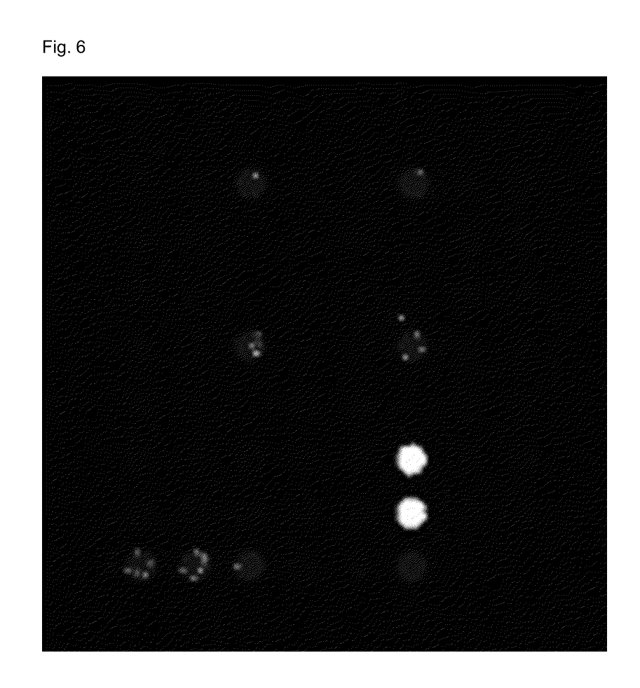

FIG. 6 shows the result of scanning only one well of the chip hybridized with the HBB forward primer-Cy3 PCR product at 532 nm.

### Example 8

### Application of DNA Microarray for HPV to Clinical Diagnosis

The HPV DNA microarray using the Y-shaped probe of the present disclosure was used for diagnosis of a cervical sample. The purposes of the test was, first, to investigate how accurately the HPV DNA chip can diagnose HPV infection and the genotype of HPV and, second, to evaluate how helpful it is in predicting cancers and important cervical lesions including precancerous lesions. For this, DNA was isolated from cervical swab samples of Korean women who were suspected of cervical HPV infection and cytopathologically diagnosed subjected to (1) test with the HPV DNA microarray of the present disclosure, (2) PCR of the HPV L1 gene followed by automated sequencing analysis, and (3) test by Hybrid Capture Assay-II (HCA-II; Digene Corporation) which is an HPV DNA test approved by the USFDA.

The HPV DNA chip of the present disclosure enables testing of all the 43 HPV types invading human cervix, anus, oral cavity, etc., whereas HCA-II tests the 12 high-risk type HPVs. Comparison was made while focusing on (1) the sensitivity and specificity of diagnosis of HPV infection, (2) the accuracy of HPV genotype diagnosis, and (3) the accuracy of prediction of cervical cancer and serious lesions including precancerous lesions. The HPV DNA microarray was prepared as described in Examples 5-7 and PCR and nucleotide sequencing were performed according to the known method (Kim K H, Yoon M S, Na Y J, Park C S, Oh M R, Moon W C. Development and evaluation of a highly sensitive human papillomavirus genotyping DNA chip. Gynecol Oncol. 2006; 100(1): 38-43). HCA-II test was performed according to the manufacturer's instructions.

The 201 subjects tested were aged between 18 and 81, and the average age was 52.4 years. The result of performing PCR of the HPV L1 gene and nucleotide sequencing is summarized in Table 3. HPV infection was identified from 191 subjects among the 201 subjects. 149 cases were high-risk HPV and 72 cases were mixed infections of more than one HPV types.

The analysis result with the HPV DNA microarray of the present disclosure was compared with that of HCA-II (Tables 4 and 5). The HPV DNA microarray of the present disclosure accurately diagnosed all (100%) the 191 cases of positive HPV infection. Among them, 174 cases (91.1%) were accurately genotyped. Although the 149 high-risk cases were accurately identified, rare types of HPV could not be identified with the chip of the present disclosure. Meanwhile, HCA-II failed to detect 40 cases of HPV from the 191 cases of HPV-positive samples and failed to detect 12 cases (8.1%) from among the 149 high-risk HPV infection samples. The HPV DNA chip of the present disclosure could accurately predict all the high-risk type cervical lesions including cervical cancer, cervical intraepithelial neoplasia (CIN) and high-grade squamous intraepithelial lesion (HSIL). In contrast, the HCA-II test failed to detect one of the 8 cases of cervical cancer and one of the 12 cases of HSIL 12. In addition, the HPV chip of the present disclosure showed better ability of detecting low-grade SIL than HCA-II (92.2%:56.9%, p<0.05, Table 6).

These results reveal that the HPV DNA chip of the present disclosure exhibits about 100% sensitivity in diagnosis of HPV infection and genotyping of HPV, especially high-risk HPV, and is excellent in predicting cervical cancer and precancerous lesion. Further, it is superior to the existing HCA-II test.

TABLE 3
Result of HPV genotyping

| Result | No Cases (%) |
|---|---|
| Total | 201 |
| Positive for HPV | 191 |
| Single infection | 119 |
| Mixed infection | 72 |
| High risk HPV | 149 (74.9) |
| Low risk HPV | 48 |
| Undetermined risk | 31 |
| Rare type | 17 |
| Individual type | — |

TABLE 4
Comparison of HPV genotyping chip with Hybrid Capture Assay-II

| | HPV DNA chip | HCA-II |
|---|---|---|
| HPV detection | 191/191 (100.0) | 151/191 (79.1) |
| High-risk HPV | 149/149 (100.0) | 137/149 (91.9) |
| detection | | |
| HPV genotyping | 174/191 (91.1)* | Impossible (not analyzable) |

The 17 types are not included in the HPV genotyping chip.

TABLE 5
Failure of test by HCA-II

| | | |
|---|---|---|
| Total | 33/171 | (19.3%) |
| High risk | 12*/149 | (8.1%) |
| Probable high risk | 5/20 | (25.0%) |
| Low risk** | 48/48 | (100.0%) |

*Types 16, 33, 35 and 68; included in HCA-II
**Not included in HCA-II

TABLE 6
Comparison of HPV genotyping chip with Hybrid Capture Assay-II for
testing of cervical cancer and precancerous lesion

| | Sensitivity | | | |
|---|---|---|---|---|
| Cytopathological diagnosis | HPV DNA Chip | | HCA-II | |
| Carcinoma | 8/8 | (100.0) | 7/8 | (87.5) |
| CIN, grade 3/3 | 1/1 | | 1/1 | |
| High grade SIL | 12/12 | (100.0) | 11/12 | (91.7) |
| Low grade SIL | 94/102 | (92.2)* | 58/102 | (56.9) |
| Carcinoma + CIN + HSIL | 20/20 | (100.0) | 19/20 | (95.0) |
| All | 115/123 | (93.5)* | 77/123 | (62.6) |

*Significantly different (p < 0.05)

### Example 9

### Development of DNA Microarray for Diagnosis of Sexually Transmitted Infection

The present disclosure provides a novel method of diagnosing sexually transmitted disease (STD) or sexually transmitted infection (STI) through genotyping using a DNA microarray on which the Y-shaped probe is spotted. This example describes another application of the Y-shaped probe and demonstrates that a DNA microarray using the Y-shaped probe is useful in diagnosis of important diseases.

Sexually transmitted infection is one of the most important diseases in humans. The incidence rate is high and the effect on quality of life and the socioeconomical effect are very high. Five out of the ten infections with the highest incidence rates in human are sexually transmitted infections. The prevalence rate is growing worldwide and it incurs great socioeconomical loss. Representative sexually transmitted diseases include Chlamydia trachomatis (CT) infection, Neisseria gonorrhoeae (NG) infection, i.e. gonorrhea, herpes simplex virus (HSV) infection, in particular genital herpes caused by HSV type 2 (HSV-2), human papilloma virus (HPV) infection, syphilis caused by Treponema pallidum (TP), chancroid caused by Haemophilus ducreyi, Trichomonas infection, acquired immunodeficiency syndrome (AIDS) caused by human immunodeficiency virus (HIV), and so forth. Among them, Chlamydia infection and Neisseria gonorrhoeae infection may cause urethritis both in men and women, epididymitis and infertility in men, and cervicitis, pelvic inflammatory disease and infertility in women. Meanwhile, syphilis, chancroid and genital herpes may cause genital ulcer (Centers for Disease Control and Prevention, USA. Sexually Transmitted Diseases. Treatment Guidelines, 2006. Morbidity and Mortality Weekly Report. Aug. 4, 2006/Vol. 55/No. RR-11).

According to the report of the US Centers for Disease Control and Prevention in 2009, Chlamydia infection has the highest prevalence rate, with 360 per 100,000 Americans infected. It is reported that the infection has increased more than 3 times in the past 20 years. It is reported that 150 out of 100,000 are infected with Neisseria gonorrhoeae. In 2008 alone, about 1.5 million patients were newly infected by CT and Neisseria gonorrhoeae. They are becoming a social concern since the incidence rate is highest in juvenile and young women aged between 15 and 24 and is increasing rapidly. The incidence rate of syphilis has declined sharply but is increasing again. In 2008 alone, about 13,500 new patients were reported. The incidence of genital herpes are increasing rapidly from 20,000 cases in 1968 to 400,000 cases in 2008. HPV infection is the sexually transmitted infection with the highest prevalence rate as a single cause and is found in 26.8% of American women between 14 and 59 years (U.S. Department of Health and Human Services. Centers for Disease Control and Prevention National Center for HIV/AIDS, Viral Hepatitis, STD, and TB. Prevention Division of STD Prevention. Sexually Transmitted Disease Surveillance 2008. Division of STD Prevention. November 2009; Centers for Disease Control and Prevention, USA. Sexually Transmitted Diseases. Treatment Guidelines, 2006. Morbidity and Mortality Weekly Report. Aug. 4, 2006/Vol. 55/No. RR-11).

What is to be noted in treatment of these sexually transmitted infections is, firstly, most of the causative pathogens require a lot of time and cost for diagnosis with the existing staining, culturing or immunological tests. Secondly, since the sexually transmitted infection is commonly a mixed infection, it is necessary to diagnose and treat multiple infections, which is not available yet. Recently, genetic testing is emerging as a new standard diagnosis test of sexually transmitted infections. For example, the PCR-based COBAS Amplicor test (Roche Diagnostic System), GenProbe APTIMA assay (Gen-Probe), real-time PCR assay (Abbott Laboratories), hybrid capture assay (Digene), and strand displacement amplification-based Becton Dickinson BD ProbeTec (Becton Dickinson), etc. are commercially available for diagnosis of Chlamydia infection and Neisseria gonorrhoeae infection. In addition, various in-house PCR test methods or PCR followed by hybridization on a microplate are employed to detect the infection. However, a genetic testing method, particularly a DNA microarray product, capable of accurately, quickly and economically testing all the pathogens causing major sexually transmitted infections is not commercially available yet. The DNA microarray may also be used to investigate drug resistance owing to mutation. Since the drug resistance is a serious problem in the treatment of sexually transmitted infections, it is important to investigate the drug resistance before selecting a drug (Cook R L, Hutchison S L, Østergaard L, Braithwaite R S, Ness R B. Systematic review: noninvasive testing for Chlamydia trachomatis and Neisseria gonorrhoeae. Annals of Internal Medicine. 2005; 142(11): 914-25; Masek B J, Arora N, Quinn N, Aumakhan B, Holden J, Hardick A, Agreda P, Barnes M, Gaydos C A. Performance of three nucleic acid amplification tests for detection of Chlamydia trachomatis and Neisseria gonorrhoeae by use of self-collected vaginal swabs obtained via an Internet-based screening program. Journal of Clinical Microbiology. 2009; 47(6): 1663-7; Gdoura R, Kchaou W, Ammar-Keskes L, Chakroun N, Sellemi A, Znazen A, Rebai T, Hammami A. Assessment of Chlamydia trachomatis, Ureaplasma urealyticum, Ureaplasma parvum, Mycoplasma hominis, and Mycoplasma genitalium in semen and first void urine specimens of asymptomatic male partners of infertile couples. Journal of Andrology. 2008; 29(2): 198-206; McKechnie M L, Hillman R, Couldwell D, Kong F, Freedman E, Wang H, Gilbert G L. Simultaneous identification of 14 genital microorganisms in urine by use of a multiplex PCR-based reverse line blot assay. J Clin Microbiol. 2009; 47(6): 1871-7; Michelle A. The laboratory diagnosis of Haemophilus ducreyi. Can J Infect Dis Med Microbiol. 200; 16(1): 31-34).

The present disclosure is directed to developing a DNA microarray capable of testing all the pathogens causing major sexually transmitted infections accurately and quickly, at minimum cost.

The DNA microarray of the present disclosure is a product on which an entirely new type of Y-shaped probe is spotted, wherein the most appropriate target genes are selected for the target pathogen and different oligonucleotide probes are disposed for the genes at two different portions. It was designed to maximize the diagnosis sensitivity by duplicately testing with two probes for one gene. Although some DNA chips for diagnosis of sexually transmitted infection have been reported, no DNA microarray of this type has been reported (Shi G, Wen S Y, Chen S H, Wang S Q. Fabrication and optimization of the multiplex PCR-based oligonucleotide microarray for detection of Neisseria gonorrhoeae, Chlamydia trachomatis and Ureaplasma urealyticum. J Microbiol Methods. 2005; 62(2): 245-56).

The DNA microarray of the present disclosure can detect all of the representative sexually transmitted diseases—Chlamydia trachomatis infection, Neisseria gonorrhoeae infection, herpes simplex virus type 2 (HSV-2) infection, Treponema pallidum infection, and chancroid caused by Haemophilus ducreyi. The present disclosure includes not only the microarray in which the Y-shaped probe for testing STD and the probe for the control reference gene are spotted, but also instructions about the PCR reagent, hybridization reagent, sampling kit and scanner. A more detailed description will be given hereinafter.

9.1. Designing of Y-Shaped Probe for Genotyping of STD-Causing Microorganisms

The DNA microarray of the present disclosure was designed to detect the five major microorganisms causing sexually transmitted diseases—Neisseria gonorrhoeae, Chlamydia trachomatis, Treponema pallidum, Haemophilus ducreyi and herpes simplex virus. For this, a Y-shaped probe was specially designed as follows.

1) Left-Side Probe Part (A in FIG. 1) and Right-Side Probe Part (E in FIG. 1)

Target genes specific for the respective pathogens were selected and amplified by PCR. Oligonucleotide probes were selected from two different regions of the PCR product and disposed on left-side and right-side probe parts. The left-side and right-side probe parts may be varied as desired.

For example, for Neisseria gonorrhoeae, the nucleotide sequence of the right-side probe part may be GAT ATT TTT CCG TAA CGT CTC TAA GTC T, and the nucleotide sequence of the left-side probe part may be CAA CAA ACG AAA GCA GAO TTA GAG ACC.

For Chlamydia trachomatis, the nucleotide sequence of the right-side probe part may be TTT TCT TCG TCA GTT AAA CCT TCC C, and the nucleotide sequence of the left-side probe part may be GTT CGT TGT AGA GCC ATG TCC TAT CC.

For herpes simplex virus 2, the nucleotide sequence of the right-side probe part may be ACC CCA CCA GCC CGG AC, and the nucleotide sequence of the left-side probe part may be GCC CCC GGG GTC GGA AGC.

For Treponema pallidum, the nucleotide sequence of the right-side probe part may be ACG TGC AGA AAA ACT ATC CTC AGT G, and the nucleotide sequence of the left-side probe part may be ACG TAA GGT AAG CAG CAT GGA GAO.

And, for Haemophilus ducreyi, the nucleotide sequence of the right-side probe part may be GTG AGT AAT GCT TGG GAA TCT GGC TT, and the nucleotide sequence of the left-side probe part may be GAA GAT ATT ACG CGG TAT TAG CTA CAC.

2) Stem Parts (B and D in FIG. 1)

The left-side stem part (B in FIG. 1) was designed with CCCTAA which is reverse to the human telomere sequence, and the right-side stem part (D in FIG. 1) was designed with TTAGGG which is complementary thereto.

3) Linker Part (C in FIG. 1)

The linker part was designed with an internal amino modifier C6 dT (iAmMC6T). The Y-shaped probe for a total of 5 microorganisms causing sexually transmitted infection was designed as described above. The probe was spotted on a glass slide as described above to fabricate a DNA chip for STD genotyping. The chip was fabricated so that as many as 8 samples could be tested. The name, SEQ ID NO and genotype of the probes are summarized in Table 7.

The strains or plasmid clones of the microorganisms were purchased from the American Type Culture Collection (ATCC) and target genes were prepared by cloning according to the known method (Table 8). Thus prepared plasmid clones of the target genes were mixed with various copy numbers and hybridized on the DNA microarray of the present disclosure.

TABLE 7
Sequence for Y-shaped probe for fabrication of STD DNA chip

| No. | Name | Sequence | Length | Strain |
|---|---|---|---|---|
| SEQ ID | NGYP | CAACAAACGAAAGCAGACTTAGAGACC | 68 | Neisseria |
| NO 51 | | CCCTAA-iAmMC6T-TTAGGGGATATTTT | | Gonorrhea, |
| | | TCCGTAACGTCTCTAAGTCT | | NG |
| SEQ ID | CTYP | GTTCGTTGTAGAGCCATGTCCTATCCC | 64 | Chlamydia |
| NO 52 | | CCTAA-iAmMC6T-TTAGGGTTTTCTTCG | | Trachomatis, |
| | | TCAGTTAAACCTTCCC | | CT |
| SEQ ID | HSVYP | GCCCCCGGGGTCGGAAGCCCCTAA-iA | 48 | Herpes |
| NO 53 | | mMC6T-TTAGGGACCCCACCAGCCCG | | simplex |
| | | GAC | | virus, HSV |
| SEQ ID | TPYP | ACGTAAGGTAAGCAGCATGGAGACCC | 62 | Treponema |
| NO 54 | | CTAA-iAmMC6T-TTAGGGACGTGCAGA | | Pallidum, TP |
| | | AAAACTATCCTCAGTG | | |
| SEQ ID | HDYP | GAAGATATTACGCGGTATTAGCTACAC | 66 | Hemophilus |
| NO 55 | | CCCTAA-iAmMC6T-TTAGGGGTGAGTA | | Ducreyi, HD |
| | | ATGCTTGGGAATCTGGCTT | | |

TABLE 8
Positive control strains and plasmid clones

| Target | | |
|---|---|---|
| microorganism | Positive control | Target gene clone |
| N. gonorrhoeae | 53420D | Plasmid pJD1 |
| C. trachomatis | VR-879 | Cryptic plasmid |
| H. ducreyi | 700724-D | 16S rRNA gene |
| T. pallidum | 632625 | carboxypeptidase gene |
| HSV | VR-733 ™ (HSV1)/ | secreted portion of |
| | VR-540 ™ (HSV2) | glycoprotein G-2(US4) |

9.2. Sample Preparation and PCR

Urine samples were obtained from men and women according to the known method. Also, cervical and vaginal swab samples were obtained from the female subjects. In addition, samples were obtained from the genital skin, particularly the ulcer region, and total DNA was isolated therefrom (Masek B J, Arora N, Quinn N, Aumakhan B, Holden J, Hardick A, Agreda P, Barnes M, Gaydos C A. Performance of three nucleic acid amplification tests for detection of Chlamydia trachomatis and Neisseria gonorrhoeae by use of self-collected vaginal swabs obtained via an Internet-based screening program. Journal of Clinical Microbiology. 2009; 47(6): 1663-7; Gdoura R, Kchaou W, Ammar-Keskes L, Chakroun N, Sellemi A, Znazen A, Rebai T, Hammami A. Assessment of Chlamydia trachomatis, Ureaplasma urealyticum, Ureaplasma parvum, Mycoplasma hominis, and Mycoplasma genitalium in semen and first void urine specimens of asymptomatic male partners of infertile couples. Journal of Andrology. 2008; 29(2): 198-206; McKechnie M L, Hillman R, Couldwell D, Kong F, Freedman E, Wang H, Gilbert G L. Simultaneous identification of 14 genital microorganisms in urine by use of a multiplex PCR-based reverse line blot assay. J Clin Microbiol. 2009; 47(6): 1871-7).

Thereafter, PCR was performed according to the known method as described below, and the PCR product was labeled with Cy5 or Cy3. The PCR was performed singly or in multiplex format. The condition is as follows.

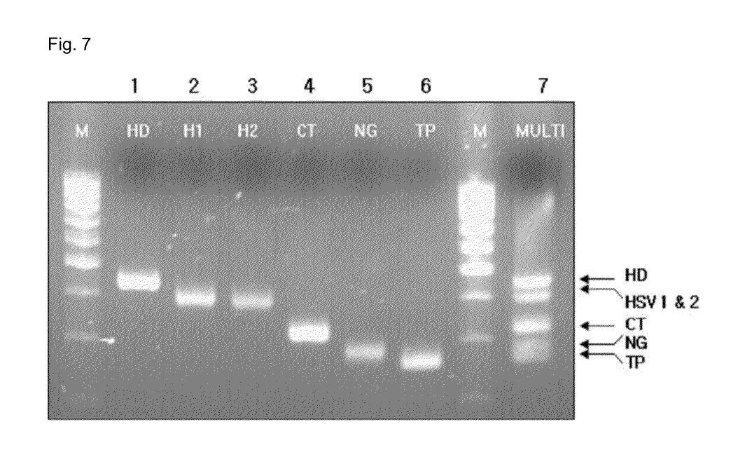

The composition of the reaction solution reaction condition for multiplex PCR are summarized in Table 9. The multiplex PCR product was identified by electrophoresis on 1.5-2.0% agarose gel. The electrophoresis image of the PCR products shows the 440-bp PCR product of Haemophilus ducreyi (HD), 384-bp PCR product of herpes simplex virus (HSV) 1, 400-bp PCR product of herpes simplex virus (HSV) 2, 321-bp PCR product of Chlamydia trachomatis (CT), 284-bp PCR product of Neisseria gonorrhoeae (NG), and 260-bp PCR product of Treponema pallidum (TP) (FIG. 7). Accordingly, it was confirmed that all the 5 pathogen genes could be detected at once by a single multiplex PCR.

TABLE 9
Condition for multiplex PCR
Composition and content of PCR solution

| | |
|---|---|
| Distilled water | 3 μl |
| A set primer, forward/reverse mixture | 4 μl |
| 2x MM Premix* | 15 μl |
| 0.05 nM Cy5-dCTP** (or Cy3-dCTP) | 2 μl |
| Template DNA (>20 ng) | 6 μl |
| Final volume | 30 μl |

PCR condition

| | | |
|---|---|---|
| Predenaturation | 95° C./10 min | 1 Cycle |
| Denaturation | 94° C./30 sec | 40 Cycles |
| Annealing | 58° C./30 sec | |
| Extention | 72° C./30 sec | |
| Final Extention | 72° C./5 min | 1 Cycle |

*2X MM premix was prepared by adding 200 μM dNTPs and 1.5 mM MgCl2 to thermally stable DNA polymerase (1 unit) and then adding distilled water to make a total volume of 15 mL.
**The ratio of Cy5-dCTP to unlabeled dCTP is 1:12.5.

9.3. Hybridization and Analysis

On the slide chip on which the oligonucleotide probes were spotted, each 10 μL of the cryptic plasmid of Neisseria gonorrhoeae and Chlamydia trachomatis and the PCR product of Haemophilus ducreyi, herpes simplex virus, Chlamydia trachomatis and Treponema pallidum genes were mixed to a final volume of 50 μL as template. After denaturation at 95° C. for 5 minutes, the mixture was immediately transferred to ice and left for 3 minutes. Then, after adding 50 μL of a hybridization solution to make the final volume 100 μL, reaction was performed at 45° C. for 30 minutes with the probes fixed on the slide. The hybridization solution was prepared by mixing 2 mL of 20×SSC with 1.7 mL of 90% glycerol and 6.3 mL of 50 mM phosphate buffer to a final volume of 10 mL.

After the hybridization was completed, the well cover was removed from the DNA chip and the chip was immersed in 3×SSPE solution (NaCl (26.295 g), NaH₂PO₄-1H₂O (4.14 g), Na₂EDTA (1.11 g) dissolved in 1 L of distilled water; adjusted to pH 7.4 with 10 N NaOH). After washing at room temperature for 2 minutes and then again with 1×SSPE solution (NaCl (8.765 g), NaH₂PO₄-1H₂O (1.38 g), Na₂EDTA (0.37 g) dissolved in 1 L of distilled water; adjusted to pH 7.4 with 10 N NaOH) at room temperature for 2 minutes, centrifugation was performed at 800 rpm at room temperature for 1.5 minutes followed by drying.

9.4. Scanning Analysis

After removal of nonspecific signals through washing, the dried slide was subjected to analysis of fluorescence signals and images using a fluorescence scanner. As for the scanner, GenePix 4000B scanner (Axon, USA), ScanArray Lite (Packard Bioscience, USA) or an equipment comparable thereto may be used.

Various copy numbers of the plasmid clones of the target gene prepared above were amplified by PCR and the product was hybridized on the DNA microarray to investigate the sensitivity of the DNA microarray. As a result of the spike testing, it was confirmed that the presence of the plasmid clones of different microorganisms in an amount of 10-100 copies per 1 mL of sample is always detectable.

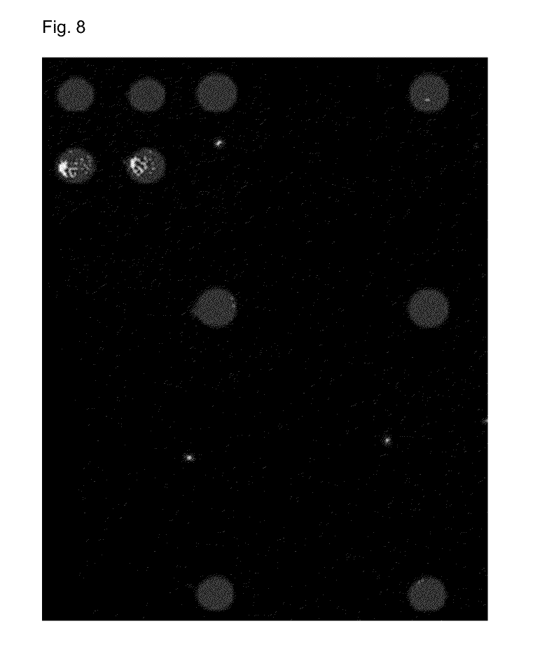

Analysis was carried out on 1252 male and 680 female Korean adults who were suspected of sexually transmitted infections between January 2008 and October 2009 using the DNA microarray of the present disclosure. Among them, 1084 cases could be compared with PCR followed by sequencing. The STD DNA microarray of the present disclosure showed a good result, with 1075 cases (99%) among them matching. FIGS. 8-12 show the result of performing hybridization on the STD chip the present disclosure and analyzing the result using a scanner as images.

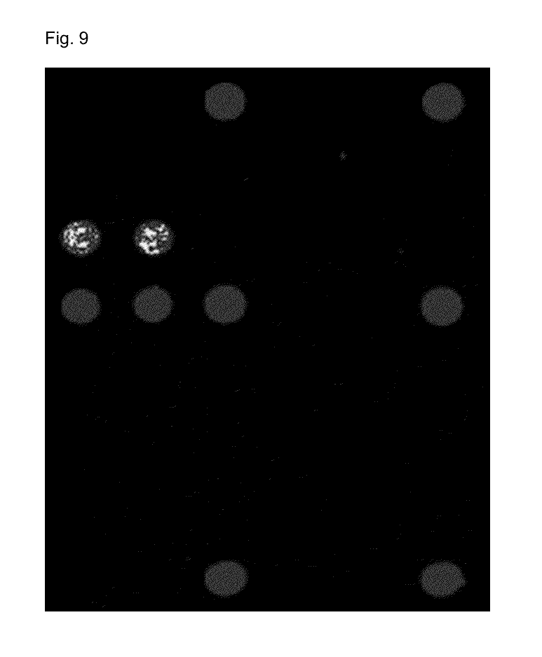

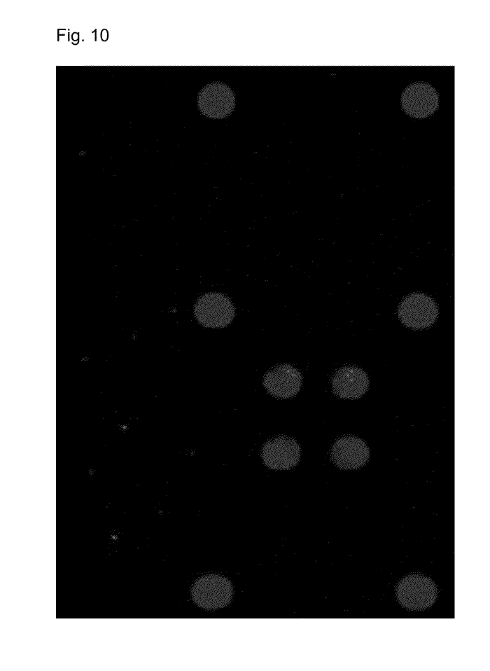

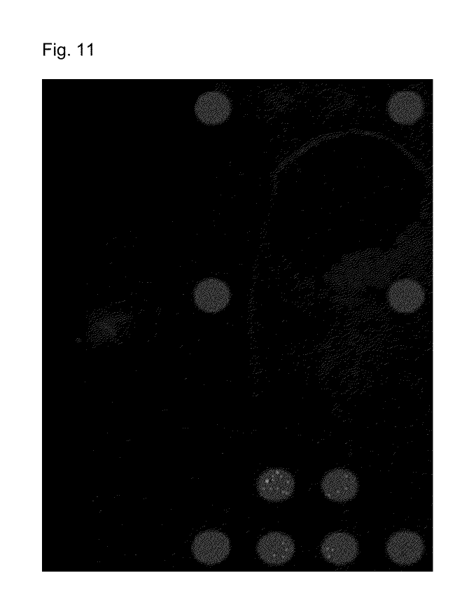

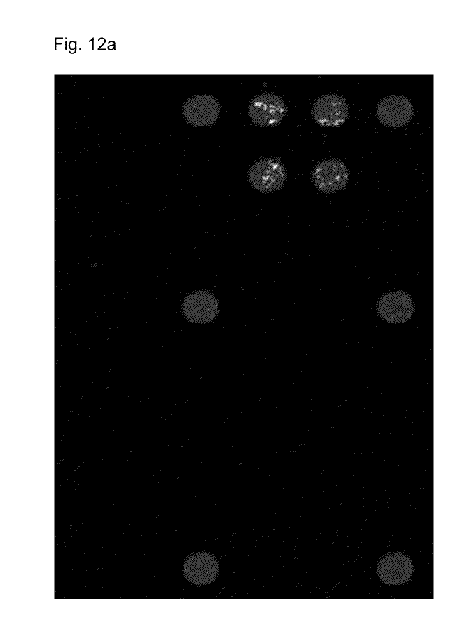

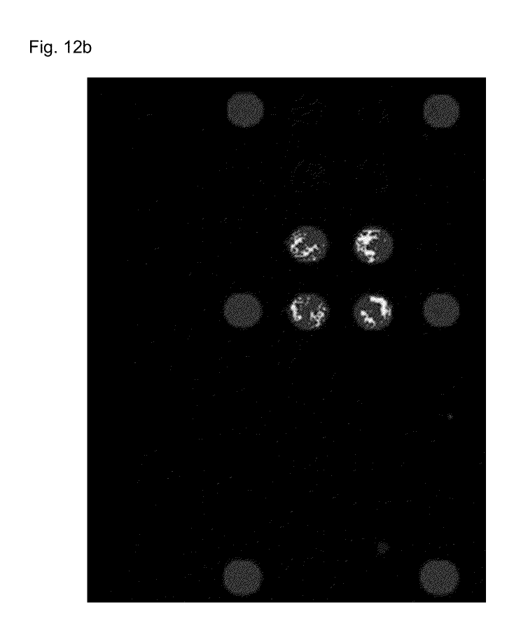

FIG. 8 shows a result of hybridizing the Y-shaped probe with Neisseria gonorrhoeae as positive substance on the STD chip. FIG. 9 shows a result of hybridizing the Y-shaped probe with Chlamydia trachomatis as positive substance on the STD chip. FIG. 10 shows a result of hybridizing the Y-shaped probe with Treponema pallidum as positive substance on the STD chip. FIG. 11 shows a result of hybridizing the Y-shaped probe with Haemophilus ducreyi as positive substance on the STD chip. And, FIG. 12 shows a result of hybridizing the Y-shaped probe with herpes simplex virus as positive substance on the STD chip.

It took about 3-4 hours until the result was obtained according to the method of the present disclosure, and about 1000 samples could be tested per day by two or three researchers using about 120 chips.

### Example 10

### Genotyping of Influenza Virus

The present disclosure provides a novel method for diagnosing influenza infection using the DNA microarray on which the Y-shaped probe is spotted and for accurately genotyping the type, subtype and strain of the causative influenza virus. This example demonstrates the applicability of the Y-shaped probe of the present disclosure to diagnosis of another important disease.

Influenza or flu is the longest disease in human history, with high incidence rate and mortality. Influenza virus invades various hosts. Since the genome of the virus consists of RNA, the virus mutates constantly (antigenic shift), causing reassortment of the genes of several species and occurrence of new variants. For this reason, there are difficulties in treatment and vaccine development (Ravi V. Emergence of novel influenza A H1N1 as a pandemic agent. Indian Journal of Medical Microbiology. 2009; 27(3): 179-181). Influenza is caused by a different type of virus from common cold. The symptom is more severe and it may develop into pneumonia since the virus invades more deeply into the respiratory organs. It can even lead to death via complications. It is epidemic between fall and winter of every year (Beers M H, Fletcher A J, Jones T V, Porter R. The Merck Manual of Medical Information. Second edition. Merck Research Laboratories. 2003: 1159-1160). According to the statistics of the US Centers for Disease Control and Prevention (CDC), more than 200,000 people are infected by influenza and 36,000 die of them every year (http://www.cdcc.gov/flu/abput/disease.htm).

There are three types of influenza virus—A, B and C. Among them, types A and B cause flu. Especially, influenza A virus is the most virulent human pathogen. The influenza virus is further classified depending on the two viral proteins hemagglutinin (HA, H) and neuraminidase (NA, N) and the gene type. There are 16 types of hemagglutinin, from hemagglutinin 1 (H1) to hemagglutinin 16 (H16), and 9 types of neuraminidase, from neuraminidase 1 (N1) to neuraminidase 9 (N9). Accordingly, the subtype of the influenza virus is denoted as H1-16N1-9. In the Influenza A virus, H1, H2, H3, N1 and N2 are mainly found. Thus, there are 6 subtypes H1-3N1-2 of influenza A viruses, and geographic origin or host name is added such as Spanish flu, Hong Kong flu or avian flu. Until now, the 3 subtypes H1N1, H2N2 and H3N2 have caused serious pandemic in humans. The Spanish flu in 1918 caused by H1N1 killed 20-50 million people worldwide. H2N2 caused a severe pandemic in 1957, and H3N2 has mainly caused problems since then. In 1998, infection of H3N2, a variant of avian flu, spread. Recently, H1N1 is causing troubles. Especially, it occurs as a mixture of human, bird and swine viruses. The swine flu (influenza A/H1N1) which is a new variant of H1N1 transmitted from pigs to humans is a severe global concern (Garten R J, Davis C T, Russell C A, Shu B, Lindstrom S, Balish A, Sessions W M, Xu X, Skepner E, Deyde V, Okomo-Adhiambo M, Gubareva L, Barnes J, Smith C B, Emery S L, Hillman M J, Rivailler P, Smagala J, de Graaf M, Burke D F, Fouchier R A, Pappas C, Alpuche-Aranda C M, Lopez-Gatell H, Olivera H, Lopez I, Myers C A, Faix D, Blair P J, Yu C, Keene K M, Dotson P D Jr, Boxrud D, Sambol A R, Abid S H, St George K, Bannerman T, Moore A L, Stringer D J, Blevins P, Demmler-Harrison G J, Ginsberg M, Kriner P, Waterman S, Smole S, Guevara H F, Belongia E A, Clark P A, Beatrice S T, Donis R, Katz J, Finelli L, Bridges C B, Shaw M, Jernigan D B, Uyeki T M, Smith D J, Klimov A I, Cox N J. Antigenic and genetic characteristics of swine-origin 2009 A (H1N1) influenza viruses circulating in humans. Science. 2009; 10; 325(5937): 197-201; Nelson M I, Viboud C, Simonsen L, Bennett R T, Griesemer S B, St George K, Taylor J, Spiro D J, Sengamalay N A, Ghedin E, Taubenberger J K, Holmes E C. Multiple reassortment events in the evolutionary history of H1N1 influenza A virus since 1918. PLoS Pathogens. 2008; 29; 4(2): e1000012; Vinknor M, Stevens J, Nawrucki J, Singh K. Influenza A virus subtyping: paradigm shift in influenza diagnosis. Journal of Clinical Microbiology. 2009; 47(9): 3055-3056; Ravi V. Emergence of novel influenza A H1N1 as a pandemic agent. Indian Journal of Medical Microbiology. 2009; 27(3): 179-181).

It is essential to accurately know the subtype of influenza virus not only for accurate diagnosis of the infection but also for prevention, treatment and epidemiological analysis. In particular, fast and accurate diagnosis is important in clinical practice. In the past, the method of culturing influenza virus and detecting the HA protein was employed for diagnosis of the virus. However, this method is being replaced by genetic testing since it requires a lot of time and cost. For example, reverse transcription PCR (RT-PCR), real-time PCR and PCR followed by enzyme-linked immunosorbent assay (ELISA) are employed. The World Health Organization (WHO) recommends real-time PCR as a standard test method of novel flu. Although these methods are useful for fast diagnosis of influenza A virus, they cannot accurately identify the subtype (Schweiger B, Zadow I, Heckler R, Timm H, Pauli G. Application of a fluorogenic PCR assay for typing and subtyping of influenza viruses in respiratory samples. J Clin Microbiol. 2000; 38(4): 1552-8; Vinknor M, Stevens J, Nawrucki J, Singh K. Influenza A virus subtyping: paradigm shift in influenza diagnosis. Journal of Clinical Microbiology. 2009; 47(9): 3055-3056; Huang Y, Tang H, Duffy S, Hong Y, Norman S, Ghosh M, He J, Bose M, Henrickson K J, Fan J, Kraft A J, Weisburg W G, Mather E L. Multiplex assay for simultaneously typing and subtyping influenza viruses by use of an electronic microarray. J Clin Microbiol. 2009; 47(2): 390-6).

In contrast, the DNA microarray enables not only diagnosis of influenza virus but also accurate identification of its subtype. In addition, the DNA microarray may be used to investigate drug resistance caused by S31N mutation of the M2 protein, genetic variation of the influenza virus, or the like. Since drug resistance is a severe problem in the treatment of influenza, it is important to investigate the drug resistance when selecting a drug (Han X, Lin X, Liu B, Hou Y, Huang J, Wu S, Liu J, Mei L, Jia G, Zhu Q. Simultaneously subtyping of all influenza A viruses using DNA microarrays. J Virol Methods. 2008; 152(1-2): 117-21; Huang Y, Tang H, Duffy S, Hong Y, Norman S, Ghosh M, He J, Bose M, Henrickson K J, Fan J, Kraft A J, Weisburg W G, Mather E L. Multiplex assay for simultaneously typing and subtyping influenza viruses by use of an electronic microarray. J Clin Microbiol. 2009; 47(2): 390-6; Nelson M I, Simonsen L, Viboud C, Miller M A, Holmes E C. The origin and global emergence of adamantane resistant A/H3N2 influenza viruses. Virology. 2009; 388(2): 270-8).

The DNA microarray using the Y-shaped probe of the present disclosure is of a new type wherein the probes for both the hemagglutinin gene and the neuraminidase gene are included in one spot. Although DNA chips for diagnosis of influenza virus were reported, a DNA microarray or a product of the same type as that of the present disclosure has never been reported (Huang Y, Tang H, Duffy S, Hong Y, Norman S, Ghosh M, He J, Bose M, Henrickson K J, Fan J, Kraft A J, Weisburg W G, Mather E L. Multiplex assay for simultaneously typing and subtyping influenza viruses by use of an electronic microarray. J Clin Microbiol. 2009; 47(2): 390-6; Lin B, Malanoski A P, Wang Z, Blaney K M, Long N C, Meador C E, Metzgar D, Myers C A, Yingst S L, Monteville M R, Saad M D, Schnur J M, Tibbetts C, Stenger D A. Universal detection and identification of avian influenza virus by use of resequencing microarrays. J Clin Microbiol. 2009; 47(4): 988-93; Han X, Lin X, Liu B, Hou Y, Huang J, Wu S, Liu J, Mei L, Jia G, Zhu Q. Simultaneously subtyping of all influenza A viruses using DNA microarrays. J Virol Methods. 2008; 152(1-2): 117-21).

The influenza DNA microarray of the present disclosure can detect all the 144 possible H1-16N1-9 types of influenza virus. The present disclosure includes not only the microarray in which 144 probes for the subtypes and the probe for the control reference gene are spotted, but also instructions about the RT-PCR reagent, hybridization reagent, sampling kit and scanner.

10.1. Designing of Y-Shaped Probe for Genotyping of Influenza Virus

The Y-shaped probe for genotyping of influenza A virus was designed as described above, based on the known nucleotide sequences of the hemagglutinin and neuraminidase genes of influenza virus (Han X, Lin X, Liu B, Hou Y, Huang J, Wu S, Liu J, Mei L, Jia G, Zhu Q. Simultaneously subtyping of all influenza A viruses using DNA microarrays. J Virol Methods. 2008; 152(1-2):117-21; Huang Y, Tang H, Duffy S, Hong Y, Norman S, Ghosh M, He J, Bose M, Henrickson K J, Fan J, Kraft A J, Weisburg W G, Mather E L. Multiplex assay for simultaneously typing and subtyping influenza viruses by use of an electronic microarray. J Clin Microbiol. 2009; 47(2): 390-6).

1) Left-Side and Right-Side Probe Parts (A and E in FIG. 1)

A probe for the neuraminidase gene was arranged on the left-side probe part (A in FIG. 1) of the Y-shaped probe, and a probe for the hemagglutinin gene was arranged on the right-side probe part (E in FIG. 1). A total of 144 probes were designed (Table 10).

2) Stem Parts (B and D in FIG. 1)

The left-side stem part (B in FIG. 1) was designed with CCCTAA which is reverse to the human telomere sequence, and the right-side stem part (D in FIG. 1) was designed with TTAGGG which is complementary thereto.

3) Linker Part (C in FIG. 1)

The linker part was designed with an internal amino modifier C6 dT (iAmMC6T). As a result, 144 Y-shaped probes were designed for the 144 influenza virus types. The probe was spotted on a glass slide as described above to fabricate a DNA chip for influenza virus genotyping. The chip was fabricated so that as many as 8 samples could be tested on one chip. The name, SEQ ID NO and genotype of the probes are summarized in Table 10.

TABLE 10
Sequence for Y-shaped probe for genotyping
of influenza A virus

| No. | Name | Sequence | Length |
|---|---|---|---|
| SEQ ID | H1/N1 | CAGAAATTCCAATTGTCAACCAACTCCCTAA-iAmMC6T- | 62 |
| NO 56 | | TTAGGGTGCTTATGTCTCTGTAGTGTCTTC | |
| SEQ ID | H1/N2 | TACAAGTTCCATTGATACAAACGCCCCTAA-iAmMC6T- | 61 |
| NO 57 | | TTAGGGTGCTTATGTCTCTGTAGTGTCTTC | |
| SEQ ID | H1/N3 | CCCTTCCAATTGTCCCTACATACCCTAA-iAmMC6T- | 59 |
| NO 58 | | TTAGGGTGCTTATGTCTCTGTAGTGTCTTC | |
| SEQ ID | H1/N4 | CAAATATCCCACTACATACATATCCCCCTAA-iAmMC6T- | 62 |
| NO 59 | | TTAGGGTGCTTATGTCTCTGTAGTGTCTTC | |
| SEQ ID | H1/N5 | CCATTCCAATTATCTCGGCAAACCCCCTAA-iAmMC6T- | 61 |
| NO 60 | | TTAGGGTGCTTATGTCTCTGTAGTGTCTTC | |
| SEQ ID | H1/N6 | TGAGTCCTTAATATATTTCCTGCCCCCTAA-iAmMC6T- | 61 |
| NO 61 | | TTAGGGTGCTTATGTCTCTGTAGTGTCTTC | |
| SEQ ID | H1/N7 | CCTGCATTAGGTATTTTCAACATCCCTAA-iAmMC6T- | 60 |
| NO 62 | | TTAGGGTGCTTATGTCTCTGTAGTGTCTTC | |
| SEQ ID | H1/N8 | CCACACATCACAATGGAGCTCCCTAA-iAmMC6T- | 57 |
| NO 63 | | TTAGGGTGCTTATGTCTCTGTAGTGTCTTC | |
| SEQ ID | H1/N9 | TTGTATAGTGTGGGTGGTGATGACCCTAA-iAmMC6T- | 60 |
| NO 64 | | TTAGGGTGCTTATGTCTCTGTAGTGTCTTC | |
| SEQ ID | H2/N1 | CAGAAATTCCAATTGTCAACCAACTCCCTAA-iAmMC6T- | 61 |
| NO 65 | | TTAGGGACATCAACACTGAATAAGAGGTC | |
| SEQ ID | H2/N2 | TACAAGTTCCATTGATACAAACGCCCCTAA-iAmMC6T- | 60 |
| NO 66 | | TTAGGGACATCAACACTGAATAAGAGGTC | |
| SEQ ID | H2/N3 | CCCTTCCAATTGTCCCTACATACCCTAA-iAmMC6T- | 58 |
| NO 67 | | TTAGGGACATCAACACTGAATAAGAGGTC | |
| SEQ ID | H2/N4 | CAAATATCCCACTACATACATATCCCCCTAA-iAmMC6T- | 61 |
| NO 68 | | TTAGGGACATCAACACTGAATAAGAGGTC | |
| SEQ ID | H2/N5 | CCATTCCAATTATCTCGGCAAACCCCCTAA-iAmMC6T- | 60 |
| NO 69 | | TTAGGGACATCAACACTGAATAAGAGGTC | |
| SEQ ID | H2/N6 | TGAGTCCTTAATATATTTCCTGCCCCCTAA-iAmMC6T- | 60 |
| NO 70 | | TTAGGGACATCAACACTGAATAAGAGGTC | |
| SEQ ID | H2/N7 | CCTGCATTAGGTATTTTCAACATCCCTAA-iAmMC6T- | 59 |
| NO 71 | | TTAGGGACATCAACACTGAATAAGAGGTC | |
| SEQ ID | H2/N8 | CCACACATCACAATGGAGCTCCCTAA-iAmMC6T- | 56 |
| NO 72 | | TTAGGGACATCAACACTGAATAAGAGGTC | |
| SEQ ID | H2/N9 | TTGTATAGTGTGGGTGGTGATGACCCTAA-iAmMC6T- | 59 |
| NO 73 | | TTAGGGACATCAACACTGAATAAGAGGTC | |
| SEQ ID | H3/N1 | CAGAAATTCCAATTGTCAACCAACTCCCTAA-iAmMC6T- | 61 |
| NO 74 | | TTAGGGCCTCGGGGTTACTTCAAAATACG | |
| SEQ ID | H3/N2 | TACAAGTTCCATTGATACAAACGCCCCTAA-iAmMC6T- | 60 |
| NO 75 | | TTAGGGCCTCGGGGTTACTTCAAAATACG | |
| SEQ ID | H3/N3 | CCCTTCCAATTGTCCCTACATACCCTAA-iAmMC6T- | 58 |
| NO 76 | | TTAGGGCCTCGGGGTTACTTCAAAATACG | |
| SEQ ID | H3/N4 | CAAATATCCCACTACATACATATCCCCCTAA-iAmMC6T- | 61 |
| NO 77 | | TTAGGGCCTCGGGGTTACTTCAAAATACG | |
| SEQ ID | H3/N5 | CCATTCCAATTATCTCGGCAAACCCCCTAA-iAmMC6T- | 60 |
| NO 78 | | TTAGGGCCTCGGGGTTACTTCAAAATACG | |
| SEQ ID | H3/N6 | TGAGTCCTTAATATATTTCCTGCCCCCTAA-iAmMC6T- | 60 |
| NO 79 | | TTAGGGCCTCGGGGTTACTTCAAAATACG | |
| SEQ ID | H3/N7 | CCTGCATTAGGTATTTTCAACATCCCTAA-iAmMC6T- | 59 |
| NO 80 | | TTAGGGCCTCGGGGTTACTTCAAAATACG | |
| SEQ ID | H3/N8 | CCACACATCACAATGGAGCTCCCTAA-iAmMC6T- | 56 |
| NO 81 | | TTAGGGCCTCGGGGTTACTTCAAAATACG | |
| SEQ ID | H3/N9 | TTGTATAGTGTGGGTGGTGATGACCCTAA-iAmMC6T- | 59 |
| NO 82 | | TTAGGGCCTCGGGGTTACTTCAAAATACG | |
| SEQ ID | H4/N1 | CAGAAATTCCAATTGTCAACCAACTCCCTAA-iAmMC6T- | 59 |
| NO 83 | | TTAGGGGACAAAGGTCAACAATGGGGA | |
| SEQ ID | H4/N2 | TACAAGTTCCATTGATACAAACGCCCCTAA-iAmMC6T- | 58 |
| NO 84 | | TTAGGGGACAAAGGTCAACAATGGGGA | |
| SEQ ID | H4/N3 | CCCTTCCAATTGTCCCTACATACCCTAA-iAmMC6T- | 56 |
| NO 85 | | TTAGGGGACAAAGGTCAACAATGGGGA | |
| SEQ ID | H4/N4 | CAAATATCCCACTACATACATATCCCCCTAA-iAmMC6T- | 59 |
| NO 86 | | TTAGGGGACAAAGGTCAACAATGGGGA | |
| SEQ ID | H4/N5 | CCATTCCAATTATCTCGGCAAACCCCCTAA-iAmMC6T- | 58 |
| NO 87 | | TTAGGGGACAAAGGTCAACAATGGGGA | |
| SEQ ID | H4/N6 | TGAGTCCTTAATATATTTCCTGCCCCCTAA-iAmMC6T- | 58 |
| NO 88 | | TTAGGGGACAAAGGTCAACAATGGGGA | |
| SEQ ID | H4/N7 | CCTGCATTAGGTATTTTCAACATCCCTAA-iAmMC6T- | 57 |
| NO 89 | | TTAGGGGACAAAGGTCAACAATGGGGA | |
| SEQ ID | H4/N8 | CCACACATCACAATGGAGCTCCCTAA-iAmMC6T- | 54 |
| NO 90 | | TTAGGGGACAAAGGTCAACAATGGGGA | |
| SEQ ID | H4/N9 | TTGTATAGTGTGGGTGGTGATGACCCTAA-iAmMC6T- | 57 |
| NO 91 | | TTAGGGGACAAAGGTCAACAATGGGGA | |
| SEQ ID | H5/N1 | CAGAAATTCCAATTGTCAACCAACTCCCTAA-iAmMC6T- | 62 |
| NO 92 | | TTAGGGGTCACCAATAAGGTCAACTCGATC | |
| SEQ ID | H5/N2 | TACAAGTTCCATTGATACAAACGCCCCTAA-iAmMC6T- | 61 |
| NO 93 | | TTAGGGGTCACCAATAAGGTCAACTCGATC | |
| SEQ ID | H5/N3 | CCCTTCCAATTGTCCCTACATACCCTAA-iAmMC6T- | 59 |
| NO 94 | | TTAGGGGTCACCAATAAGGTCAACTCGATC | |
| SEQ ID | H5/N4 | CAAATATCCCACTACATACATATCCCCCTAA-iAmMC6T- | 62 |
| NO 95 | | TTAGGGGTCACCAATAAGGTCAACTCGATC | |
| SEQ ID | H5/N5 | CCATTCCAATTATCTCGGCAAACCCCCTAA-iAmMC6T- | 61 |
| NO 96 | | TTAGGGGTCACCAATAAGGTCAACTCGATC | |
| SEQ ID | H5/N6 | TGAGTCCTTAATATATTTCCTGCCCCCTAA-iAmMC6T- | 61 |
| NO 97 | | TTAGGGGTCACCAATAAGGTCAACTCGATC | |
| SEQ ID | H5/N7 | CCTGCATTAGGTATTTTCAACATCCCTAA-iAmMC6T- | 60 |
| NO 98 | | TTAGGGGTCACCAATAAGGTCAACTCGATC | |
| SEQ ID | H5/N8 | CCACACATCACAATGGAGCTCCCTAA-iAmMC6T- | 57 |
| NO 99 | | TTAGGGGTCACCAATAAGGTCAACTCGATC | |
| SEQ ID | H5/N9 | TTGTATAGTGTGGGTGGTGATGACCCTAA-iAmMC6T- | 60 |
| NO 100 | | TTAGGGGTCACCAATAAGGTCAACTCGATC | |
| SEQ ID | H6/N1 | CAGAAATTCCAATTGTCAACCAACTCCCTAA-iAmMC6T- | 63 |
| NO 101 | | TTAGGGTGAGATGTTTCCCAAAAGTACATGG | |
| SEQ ID | H6/N2 | TACAAGTTCCATTGATACAAACGCCCCTAA-iAmMC6T- | 62 |
| NO 102 | | TTAGGGTGAGATGTTTCCCAAAAGTACATGG | |
| SEQ ID | H6/N3 | CCCTTCCAATTGTCCCTACATACCCTAA-iAmMC6T- | 60 |
| NO 103 | | TTAGGGTGAGATGTTTCCCAAAAGTACATGG | |
| SEQ ID | H6/N4 | CAAATATCCCACTACATACATATCCCCCTAA-iAmMC6T- | 63 |
| NO 104 | | TTAGGGTGAGATGTTTCCCAAAAGTACATGG | |
| SEQ ID | H6/N5 | CCATTCCAATTATCTCGGCAAACCCCCTAA-iAmMC6T- | 62 |
| NO 105 | | TTAGGGTGAGATGTTTCCCAAAAGTACATGG | |
| SEQ ID | H6/N6 | TGAGTCCTTAATATATTTCCTGCCCCCTAA-iAmMC6T- | 62 |
| NO 106 | | TTAGGGTGAGATGTTTCCCAAAAGTACATGG | |
| SEQ ID | H6/N7 | CCTGCATTAGGTATTTTCAACATCCCTAA-iAmMC6T- | 61 |
| NO 107 | | TTAGGGTGAGATGTTTCCCAAAAGTACATGG | |
| SEQ ID | H6/N8 | CCACACATCACAATGGAGCTCCCTAA-iAmMC6T- | 58 |
| NO 108 | | TTAGGGTGAGATGTTTCCCAAAAGTACATGG | |
| SEQ ID | H6/N9 | TTGTATAGTGTGGGTGGTGATGACCCTAA-iAmMC6T- | 61 |
| NO 109 | | TTAGGGTGAGATGTTTCCCAAAAGTACATGG | |
| SEQ ID | H7/N1 | CAGAAATTCCAATTGTCAACCAACTCCCTAA-iAmMC6T- | 62 |
| NO 110 | | TTAGGGCAGACCAAACTCTATGGAAGTGGA | |
| SEQ ID | H7/N2 | TACAAGTTCCATTGATACAAACGCCCCTAA-iAmMC6T- | 61 |
| NO 111 | | TTAGGGCAGACCAAACTCTATGGAAGTGGA | |
| SEQ ID | H7/N3 | CCCTTCCAATTGTCCCTACATACCCTAA-iAmMC6T- | 59 |
| NO 112 | | TTAGGGCAGACCAAACTCTATGGAAGTGGA | |
| SEQ ID | H7/N4 | CAAATATCCCACTACATACATATCCCCCTAA-iAmMC6T- | 62 |
| NO 113 | | TTAGGGCAGACCAAACTCTATGGAAGTGGA | |
| SEQ ID | H7/N5 | CCATTCCAATTATCTCGGCAAACCCCCTAA-iAmMC6T- | 61 |
| NO 114 | | TTAGGGCAGACCAAACTCTATGGAAGTGGA | |
| SEQ ID | H7/N6 | TGAGTCCTTAATATATTTCCTGCCCCCTAA-iAmMC6T- | 61 |
| NO 115 | | TTAGGGCAGACCAAACTCTATGGAAGTGGA | |
| SEQ ID | H7/N7 | CCTGCATTAGGTATTTTCAACATCCCTAA-iAmMC6T- | 60 |
| NO 116 | | TTAGGGCAGACCAAACTCTATGGAAGTGGA | |
| SEQ ID | H7/N8 | CCACACATCACAATGGAGCTCCCTAA-iAmMC6T- | 57 |
| NO 117 | | TTAGGGCAGACCAAACTCTATGGAAGTGGA | |
| SEQ ID | H7/N9 | TTGTATAGTGTGGGTGGTGATGACCCTAA-iAmMC6T- | 60 |
| NO 118 | | TTAGGGCAGACCAAACTCTATGGAAGTGGA | |
| SEQ ID | H8/N1 | CAGAAATTCCAATTGTCAACCAACTCCCTAA-iAmMC6T- | 61 |
| NO 119 | | TTAGGGTGGAGACATCATTTTCTTATGGG | |
| SEQ ID | H8/N2 | TACAAGTTCCATTGATACAAACGCCCCTAA-iAmMC6T- | 60 |
| NO 120 | | TTAGGGTGGAGACATCATTTTCTTATGGG | |
| SEQ ID | H8/N3 | CCCTTCCAATTGTCCCTACATACCCTAA-iAmMC6T- | 58 |
| NO 121 | | TTAGGGTGGAGACATCATTTTCTTATGGG | |
| SEQ ID | H8/N4 | CAAATATCCCACTACATACATATCCCCCTAA-iAmMC6T- | 61 |
| NO 122 | | TTAGGGTGGAGACATCATTTTCTTATGGG | |
| SEQ ID | H8/N5 | CCATTCCAATTATCTCGGCAAACCCCCTAA-iAmMC6T- | 60 |
| NO 123 | | TTAGGGTGGAGACATCATTTTCTTATGGG | |
| SEQ ID | H8/N6 | TGAGTCCTTAATATATTTCCTGCCCCCTAA-iAmMC6T- | 60 |
| NO 124 | | TTAGGGTGGAGACATCATTTTCTTATGGG | |
| SEQ ID | H8/N7 | CCTGCATTAGGTATTTTCAACATCCCTAA-iAmMC6T- | 59 |
| NO 125 | | TTAGGGTGGAGACATCATTTTCTTATGGG | |
| SEQ ID | H8/N8 | CCACACATCACAATGGAGCTCCCTAA-iAmMC6T- | 56 |
| NO 126 | | TTAGGGTGGAGACATCATTTTCTTATGGG | |
| SEQ ID | H8/N9 | TTGTATAGTGTGGGTGGTGATGACCCTAA-iAmMC6T- | 59 |
| NO 127 | | TTAGGGTGGAGACATCATTTTCTTATGGG | |
| SEQ ID | H9/N1 | CAGAAATTCCAATTGTCAACCAACTCCCTAA-iAmMC6T- | 62 |
| NO 128 | | TTAGGGCAAGACGCCCAATACACAAATAAT | |
| SEQ ID | H9/N2 | TACAAGTTCCATTGATACAAACGCCCCTAA-iAmMC6T- | 61 |
| NO 129 | | TTAGGGCAAGACGCCCAATACACAAATAAT | |
| SEQ ID | H9/N3 | CCCTTCCAATTGTCCCTACATACCCTAA-iAmMC6T- | 59 |
| NO 130 | | TTAGGGCAAGACGCCCAATACACAAATAAT | |
| SEQ ID | H9/N4 | CAAATATCCCACTACATACATATCCCCCTAA-iAmMC6T- | 62 |
| NO 131 | | TTAGGGCAAGACGCCCAATACACAAATAAT | |
| SEQ ID | H9/N5 | CCATTCCAATTATCTCGGCAAACCCCCTAA-iAmMC6T- | 61 |
| NO 132 | | TTAGGGCAAGACGCCCAATACACAAATAAT | |
| SEQ ID | H9/N6 | TGAGTCCTTAATATATTTCCTGCCCCCTAA-iAmMC6T- | 61 |
| NO 133 | | TTAGGGCAAGACGCCCAATACACAAATAAT | |
| SEQ ID | H9/N7 | CCTGCATTAGGTATTTTCAACATCCCTAA-iAmMC6T- | 60 |
| NO 134 | | TTAGGGCAAGACGCCCAATACACAAATAAT | |
| SEQ ID | H9/N8 | CCACACATCACAATGGAGCTCCCTAA-iAmMC6T- | 57 |
| NO 135 | | TTAGGGCAAGACGCCCAATACACAAATAAT | |
| SEQ ID | H9/N9 | TTGTATAGTGTGGGTGGTGATGACCCTAA-iAmMC6T- | 60 |
| NO 136 | | TTAGGGCAAGACGCCCAATACACAAATAAT | |
| SEQ ID | H10/N1 | CAGAAATTCCAATTGTCAACCAACTCCCTAA-iAmMC6T- | 61 |
| NO 137 | | TTAGGGAAAACAACTTTGTGCCTGTGGT | |
| SEQ ID | H10/N2 | TACAAGTTCCATTGATACAAACGCCCCTAA-iAmMC6T- | 60 |
| NO 138 | | TTAGGGAAAACAACTTTGTGCCTGTGGT | |
| SEQ ID | H10/N3 | CCCTTCCAATTGTCCCTACATACCCTAA-iAmMC6T- | 58 |
| NO 139 | | TTAGGGAAAACAACTTTGTGCCTGTGGT | |
| SEQ ID | H10/N4 | CAAATATCCCACTACATACATATCCCCCTAA-iAmMC6T- | 61 |
| NO 140 | | TTAGGGAAAACAACTTTGTGCCTGTGGT | |
| SEQ ID | H10/N5 | CCATTCCAATTATCTCGGCAAACCCCCTAA-iAmMC6T- | 60 |
| NO 141 | | TTAGGGAAAACAACTTTGTGCCTGTGGT | |
| SEQ ID | H10/N6 | TGAGTCCTTAATATATTTCCTGCCCCCTAA-iAmMC6T- | 60 |
| NO 142 | | TTAGGGAAAACAACTTTGTGCCTGTGGT | |
| SEQ ID | H10/N7 | CCTGCATTAGGTATTTTCAACATCCCTAA-iAmMC6T- | 59 |
| NO 143 | | TTAGGGAAAACAACTTTGTGCCTGTGGT | |
| SEQ ID | H10/N8 | CCACACATCACAATGGAGCTCCCTAA-iAmMC6T- | 56 |
| NO 144 | | TTAGGGAAAACAACTTTGTGCCTGTGGT | |
| SEQ ID | H10/N9 | TTGTATAGTGTGGGTGGTGATGACCCTAA-iAmMC6T- | 59 |
| NO 145 | | TTAGGGAAAACAACTTTGTGCCTGTGGT | |
| SEQ ID | H11/N1 | CAGAAATTCCAATTGTCAACCAACTCCCTAA-iAmMC6T- | 64 |
| NO 146 | | TTAGGGCAGTGAAATAGAGGAGAGGATAA | |
| | | ACC | |
| SEQ ID | H11/N2 | TACAAGTTCCATTGATACAAACGCCCCTAA-iAmMC6T- | 63 |
| NO 147 | | TTAGGGCAGTGAAATAGAGGAGAGGATAAACC | |
| SEQ ID | H11/N3 | CCCTTCCAATTGTCCCTACATACCCTAA-iAmMC6T- | 61 |
| NO 148 | | TTAGGGCAGTGAAATAGAGGAGAGGATAAACC | |
| SEQ ID | H11/N4 | CAAATATCCCACTACATACATATCCCCCTAA-iAmMC6T- | 64 |
| NO 149 | | TTAGGGCAGTGAAATAGAGGAGAGGATAAACC | |
| SEQ ID | H11/N5 | CCATTCCAATTATCTCGGCAAACCCCCTAA-iAmM | 63 |
| NO 150 | | C6T-TTAGGGCAGTGAAATAGAGGAGAGGATAAA | |
| | | CC | |
| SEQ ID | H11/N6 | TGAGTCCTTAATATATTTCCTGCCCCCTAA-iAmMC6T- | 63 |
| NO 151 | | TTAGGGCAGTGAAATAGAGGAGAGGATAAACC | |
| SEQ ID | H11/N7 | CCTGCATTAGGTATTTTCAACATCCCTAA-iAmMC6T- | 62 |
| NO 152 | | TTAGGGCAGTGAAATAGAGGAGAGGATAAACC | |
| SEQ ID | H11/N8 | CCACACATCACAATGGAGCTCCCTAA-iAmMC6T- | 59 |
| NO 153 | | TTAGGGCAGTGAAATAGAGGAGAGGATAAACC | |
| SEQ ID | H11/N9 | TTGTATAGTGTGGGTGGTGATGACCCTAA-iAmMC6T- | 62 |
| NO 154 | | TTAGGGCAGTGAAATAGAGGAGAGGATAAACC | |
| SEQ ID | H12.N1 | CAGAAATTCCAATTGTCAACCAACTCCCTAA-iAmMC6T- | 61 |
| NO 155 | | TTAGGGTAATCACAGGGAAATCACATGGC | |
| SEQ ID | H12.N2 | TACAAGTTCCATTGATACAAACGCCCCTAA-iAmMC6T- | 60 |
| NO 156 | | TTAGGGTAATCACAGGGAAATCACATGGC | |
| SEQ ID | H12.N3 | CCCTTCCAATTGTCCCTACATACCCTAA-iAmMC6T- | 58 |
| NO 157 | | TTAGGGTAATCACAGGGAAATCACATGGC | |
| SEQ ID | H12.N4 | CAAATATCCCACTACATACATATCCCCCTAA-iAmMC6T- | 61 |
| NO 158 | | TTAGGGTAATCACAGGGAAATCACATGGC | |
| SEQ ID | H12.N5 | CCATTCCAATTATCTCGGCAAACCCCCTAA-iAmMC6T- | 60 |
| NO 159 | | TTAGGGTAATCACAGGGAAATCACATGGC | |
| SEQ ID | H12.N6 | TGAGTCCTTAATATATTTCCTGCCCCCTAA-iAmMC6T- | 60 |
| NO 160 | | TTAGGGTAATCACAGGGAAATCACATGGC | |
| SEQ ID | H12.N7 | CCTGCATTAGGTATTTTCAACATCCCTAA-iAmMC6T- | 59 |
| NO 161 | | TTAGGGTAATCACAGGGAAATCACATGGC | |
| SEQ ID | H12.N8 | CCACACATCACAATGGAGCTCCCTAA-iAmMC6T- | 56 |
| NO 162 | | TTAGGGTAATCACAGGGAAATCACATGGC | |
| SEQ ID | H12.N9 | TTGTATAGTGTGGGTGGTGATGACCCTAA-iAmMC6T- | 59 |
| NO 163 | | TTAGGGTAATCACAGGGAAATCACATGGC | |
| SEQ ID | H13/N1 | CAGAAATTCCAATTGTCAACCAACTCCCTAA-iAmMC6T- | 64 |
| NO 164 | | TTAGGGGGATGAAGATTTACTGGTATTTGATG | |
| SEQ ID | H13/N2 | TACAAGTTCCATTGATACAAACGCCCCTAA-iAmMC6T- | 63 |
| NO 165 | | TTAGGGGGATGAAGATTTACTGGTATTTGATG | |
| SEQ ID | H13/N3 | CCCTTCCAATTGTCCCTACATACCCTAA-iAmMC6T- | 61 |
| NO 166 | | TTAGGGGGATGAAGATTTACTGGTATTTGATG | |
| SEQ ID | H13/N4 | CAAATATCCCACTACATACATATCCCCCTAA-iAmMC6T- | 64 |
| NO 167 | | TTAGGGGGATGAAGATTTACTGGTATTTGATG | |
| SEQ ID | H13/N5 | CCATTCCAATTATCTCGGCAAACCCCCTAA-iAmMC6T- | 63 |
| NO 168 | | TTAGGGGGATGAAGATTTACTGGTATTTGATG | |
| SEQ ID | H13/N6 | TGAGTCCTTAATATATTTCCTGCCCCCTAA-iAmMC6T- | 63 |
| NO 169 | | TTAGGGGGATGAAGATTTACTGGTATTTGATG | |
| SEQ ID | H13/N7 | CCTGCATTAGGTATTTTCAACATCCCTAA-iAmMC6T- | 62 |
| NO 170 | | TTAGGGGGATGAAGATTTACTGGTATTTGATG | |
| SEQ ID | H13/N8 | CCACACATCACAATGGAGCTCCCTAA-iAmMC6T- | 59 |
| NO 171 | | TTAGGGGGATGAAGATTTACTGGTATTTGATG | |
| SEQ ID | H13/N9 | TTGTATAGTGTGGGTGGTGATGACCCTAA-iAmMC6T- | 62 |
| NO 172 | | TTAGGGGGATGAAGATTTACTGGTATTTGATG | |
| SEQ ID | H14/N1 | CAGAAATTCCAATTGTCAACCAACTCCCTAA-iAmMC6T- | 61 |
| NO 173 | | TTAGGGCCATCAAGCGATAATGAGCAAAC | |
| SEQ ID | H14/N2 | TACAAGTTCCATTGATACAAACGCCCCTAA-iAmMC6T- | 60 |
| NO 174 | | TTAGGGCCATCAAGCGATAATGAGCAAAC | |
| SEQ ID | H14/N3 | CCCTTCCAATTGTCCCTACATACCCTAA-iAmMC6T- | 58 |
| NO 175 | | TTAGGGCCATCAAGCGATAATGAGCAAAC | |
| SEQ ID | H14/N4 | CAAATATCCCACTACATACATATCCCCCTAA-iAmMC6T- | 61 |
| NO 176 | | TTAGGGCCATCAAGCGATAATGAGCAAAC | |
| SEQ ID | H14/N5 | CCATTCCAATTATCTCGGCAAACCCCCTAA-iAmMC6T- | 60 |
| NO 177 | | TTAGGGCCATCAAGCGATAATGAGCAAAC | |
| SEQ ID | H14/N6 | TGAGTCCTTAATATATTTCCTGCCCCCTAA-iAmMC6T- | 60 |
| NO 178 | | TTAGGGCCATCAAGCGATAATGAGCAAAC | |
| SEQ ID | H14/N7 | CCTGCATTAGGTATTTTCAACATCCCTAA-iAmMC6T- | 59 |
| NO 179 | | TTAGGGCCATCAAGCGATAATGAGCAAAC | |
| SEQ ID | H14/N8 | CCACACATCACAATGGAGCTCCCTAA-iAmMC6T- | 56 |
| NO 180 | | TTAGGGCCATCAAGCGATAATGAGCAAAC | |
| SEQ ID | H14/N9 | TTGTATAGTGTGGGTGGTGATGACCCTAA-iAmMC6T- | 59 |
| NO 181 | | TTAGGGCCATCAAGCGATAATGAGCAAAC | |
| SEQ ID | H15/N1 | CAGAAATTCCAATTGTCAACCAACTCCCTAA-iAmMC6T- | 62 |
| NO 182 | | TTAGGGGCATACAATTGACCTTGCAGATTC | |
| SEQ ID | H15/N2 | TACAAGTTCCATTGATACAAACGCCCCTAA-iAmMC6T- | 61 |
| NO 183 | | TTAGGGGCATACAATTGACCTTGCAGATTC | |
| SEQ ID | H15/N3 | CCCTTCCAATTGTCCCTACATACCCTAA-iAmMC6T- | 59 |
| NO 184 | | TTAGGGGCATACAATTGACCTTGCAGATTC | |
| SEQ ID | H15/N4 | CAAATATCCCACTACATACATATCCCCCTAA-iAmMC6T- | 62 |
| NO 185 | | TTAGGGGCATACAATTGACCTTGCAGATTC | |
| SEQ ID | H15/N5 | CCATTCCAATTATCTCGGCAAACCCCCTAA-iAmMC6T- | 61 |
| NO 186 | | TTAGGGGCATACAATTGACCTTGCAGATTC | |
| SEQ ID | H15/N6 | TGAGTCCTTAATATATTTCCTGCCCCCTAA-iAmMC6T- | 61 |
| NO 187 | | TTAGGGGCATACAATTGACCTTGCAGATTC | |
| SEQ ID | H15/N7 | CCTGCATTAGGTATTTTCAACATCCCTAA-iAmMC6T- | 60 |
| NO 188 | | TTAGGGGCATACAATTGACCTTGCAGATTC | |
| SEQ ID | H15/N8 | CCACACATCACAATGGAGCTCCCTAA-iAmMC6T- | 57 |
| NO 189 | | TTAGGGGCATACAATTGACCTTGCAGATTC | |
| SEQ ID | H15/N9 | TTGTATAGTGTGGGTGGTGATGACCCTAA-iAmMC6T- | 60 |
| NO 190 | | TTAGGGGCATACAATTGACCTTGCAGATTC | |
| SEQ ID | H16/N1 | CAGAAATTCCAATTGTCAACCAACTCCCTAA-iAmMC6T- | 62 |
| NO 191 | | TTAGGGGACAGAACATTAGACCTGCATGAT | |
| SEQ ID | H16/N2 | TACAAGTTCCATTGATACAAACGCCCCTAA-iAmMC6T- | 61 |
| NO 192 | | TTAGGGGACAGAACATTAGACCTGCATGAT | |
| SEQ ID | H16/N3 | CCCTTCCAATTGTCCCTACATACCCTAA-iAmMC6T- | 59 |
| NO 193 | | TTAGGGGACAGAACATTAGACCTGCATGAT | |
| SEQ ID | H16/N4 | CAAATATCCCACTACATACATATCCCCCTAA-iAmMC6T- | 62 |
| NO 194 | | TTAGGGGACAGAACATTAGACCTGCATGAT | |
| SEQ ID | H16/N5 | CCATTCCAATTATCTCGGCAAACCCCCTAA-iAmMC6T- | 61 |
| NO 195 | | TTAGGGGACAGAACATTAGACCTGCATGAT | |
| SEQ ID | H16/N6 | TGAGTCCTTAATATATTTCCTGCCCCCTAA-iAmMC6T- | 61 |
| NO 196 | | TTAGGGGACAGAACATTAGACCTGCATGAT | |
| SEQ ID | H16/N7 | CCTGCATTAGGTATTTTCAACATCCCTAA-iAmMC6T- | 60 |
| NO 197 | | TTAGGGGACAGAACATTAGACCTGCATGAT | |
| SEQ ID | H16/N8 | CCACACATCACAATGGAGCTCCCTAA-iAmMC6T- | 57 |
| NO 198 | | TTAGGGGACAGAACATTAGACCTGCATGAT | |
| SEQ ID | H16/N9 | TTGTATAGTGTGGGTGGTGATGACCCTAA-iAmMC6T- | 60 |
| NO 199 | | TTAGGGGACAGAACATTAGACCTGCATGAT | |

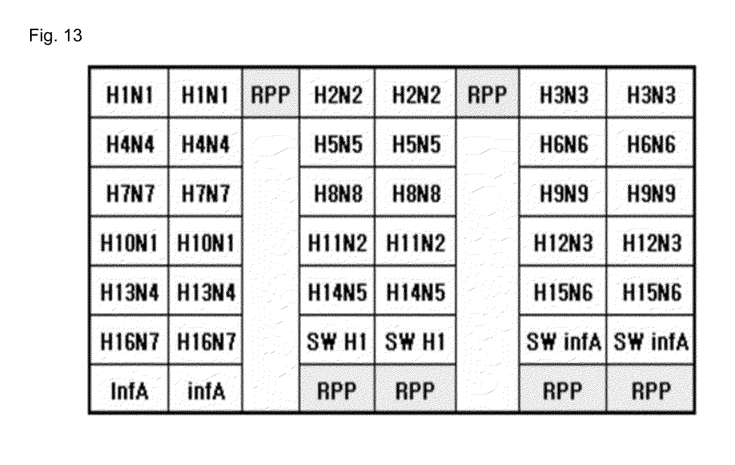

Among the Y-shaped probes described in Table 10, fifteen probes of SEQ ID NOS 56 (H1N1), 57 (H1N2), 65 (H2N1), 66 (H2N2), 74 (H3N1), 75 (H3N2), 76 (H3N3), 86 (H4N4), 96 (H5N5), 106 (H6N6), 116 (H7N7), 126 (H8N8), 136 (H9N9), 137 (H10N1) and 147 (H11N2) were prepared. Also, four linear, single-stranded probes were prepared for as control gene probes. They include a human influenza probe (inf A): 5′-C6 amine linker-TGC AGT CCT CGC TCA CTG GGC ACG-3′, a swine influenza probe (SW inf A): 5′-C6 amine linker-CYA CTG CAA GCC CAT ACA CAC AAG CAG GCA-3′, a probe for the swine influenza H1 gene (SW H1): 5′-C6 amine linker-CA GAA TAT ACA TCC RGT CAC AAT TGG ARA A-3′, and a probe for the RNase P gene: 5′-C6 amine linker-TTC TGA CCT GAA GGC TCT GCG CG-3′. These probes were spotted on an aldehyde-coated glass slide according to the method described in Example 4 to fabricate a chip for diagnosis of influenza A virus. FIG. 13 shows grids of the chip of the present disclosure.

10.2. Sample Collection and Treatment and RT-PCR

RNA was isolated from samples obtained from the upper respiratory tract of patients suspected of infection with influenza, particularly swine flu A H1/N1, according to a known method. Then RT-PCR and real-time PCR were carried out according to the method published in “CDC protocol of real time RT-PCR for swine influenza virus A (H1N1)” on Apr. 30, 2009 by the WHO (Schweiger B, Zadow I, Heckler R, Timm H, Pauli G. Application of a fluorogenic PCR assay for typing and subtyping of influenza viruses in respiratory samples. J Clin Microbiol. 2000; 38(4): 1552-8; USA Center for Disease Control and Prevention. CDC swine influenza real-time RT-PCR detection panel with the Roche LightCycler 2.0 real-time PCR system. Instruction for Use. 2009).

Nasopharyngeal aspirate, nasopharyngeal swab and throat swab samples were collected in a sample collection tube pretreated with the RNase inhibitor DEPC. Then, RNA was isolated therefrom using the QiaAmp viral RNA minikit (Qiagen Inc, USA.). The RNA was subjected to RT-PCR using the SuperScript III Platinum one-step quantitative kit (Invitrogen Inc., USA) and PCR primers for the HA and NA genes. The PCR primer for the HA gene was labeled with Cy5, and the PCR primer for the NA gene was labeled with Cy3. And, primers for RPP, SWH1, SW infA and infA were labeled with Cy5. PCR of the HA and NA genes was carried out simultaneously in duplex format under the following conditions. Detailed description will be given hereinafter.

10.2.1. Extraction of Viral PNA

Viral PNA was extracted according to a known method as follows.

1) Preparation of buffer

\textcircled{1} Add 1 mL of AVL buffer to a tube holding lyophilized carrier RNA. Dissolve the carrier RNA and add AVL buffer again. After the addition of the carrier RNA, the tube is kept at 4° C.,

\textcircled{2} Add 100% ethanol to a container holding AW1 and AW2 buffers.

2) After all the buffers and sample (VTM) are prepared, add 560 μL of AVL buffer to a 1.5-mL tube.

3) Add 140 μL of sample and mix for about 10 seconds using a vortexer. Then, collect the sample adhering to the cover or wall of the tube by spinning down.

4) Stabilize at room temperature (about 24° C.) for 10 minutes.

5) Add 560 μL of 96-100% ethanol and mix for about 10 seconds using a vortexer. Then, collect the sample adhering to the cover or wall of the tube by spinning down.

6) Add 630 μL of sample to a spin column and centrifuge at 8,000 rpm for 1 minute. Replace the collection tube with a fresh one.

7) Repeat the step 6) once again.

8) Add 500 μL of AW1 buffer and centrifuge at 8,000 rpm for 1 minute.

9) Add 500 μL of AW2 buffer and centrifuge at 14,000 rpm for 3 minutes.

10) Load the spin column on a fresh 1.5-mL tube and cautiously add 60 μL of AVE buffer to the membrane in the column. Stabilize for 1 minute and centrifuge at 8,000 rpm for 1 minute.

10.2.2. Real-Time One-Step RT-PCR

Real-time RT-PCR was performed as follows using the SuperScript III Platinum one-step quantitative kit (Invitrogen, Cat. No 11745) according to the manufacturer's instructions. Oligonucleotides having the nucleotide sequences published by the WHO were used as PCR primers (Table 11).

TABLE 11
Sequence of primers for real-time PCR

| | | | | | Size of |
|---|---|---|---|---|---|
| | | Sequence | | GC | primer |
| No | Name | (5′->3′) | Tm | % | (bp) |
| SEQ ID | InfA F | GAC CRA TCC TGT CAC | 63.6-66 | 56.81 | 22 |
| NO 200 | | CTC TGA C | | | |
| SEQ ID | InfA R | AGG GCA TTY TGG ACA | 67.1-69.4 | 45.83 | 24 |
| NO 201 | | AAK CGT CTA | | | |
| SEQ ID | SW | GCA CGG TCA GCA CTT | 62.7-67.9 | 52.17 | 23 |
| NO 202 | InfA F | ATY CTR AG | | | |
| SEQ ID | SW | GTG RGC TGG GTT TTC | 66.5-70 | 50 | 23 |
| NO 203 | InfA R | ATT TGG TC | | | |
| SEQ ID | SW | GTG CTA TAA ACA CCA | 64.9-66.9 | 50 | 23 |
| NO 204 | H1 F | GCC TYC CA | | | |
| SEQ ID | SW | CGG GAT ATT CCT TAA | 63.4-67.8 | 47.91 | 24 |
| NO 205 | H1 R | TCC TGT RGC | | | |
| SEQ ID | RNase | AGA TTT GGA CCT GCG | 66.5 | 57.89 | 19 |
| NO 206 | P F | AGC G | | | |
| SEQ ID | RNase | GAG CGG CTG TCT CCA | 65.7 | 60 | 20 |
| NO 207 | P R | CAA GT | | | |

(R stands for G or A and K stands for G or T.)

1) Prepare a master mixture for each of the oligonucleotides in Table 11 and spin down after mixing well with a pipette.

Master Mixture

| Contents | Volume | |
|---|---|---|
| Taq & RT-mix | N × 0.5 | μl |
| 2x PCR MM | N × 12.5 | μl |
| 10 μM Probe | N × 0.5 | μl |
| 40 μM Primer (F + R) | N × 0.5 | μl |
| Rnase-free water | N × 6 | μl |
| Total volume | 25 | μl |

Note)
N: sample number,

2) Transfer 20 μL to each tube and sequentially add 5 μL of negative control, sample viral RNA and positive control to the tube.

3) Load the tube on the rotor of a real-time PCR instrument and perform PCR under the conditions described in the following table.

Real time Profile

| Step | Temp./time | Cycles |
|---|---|---|
| Reverse transcription | 50° C./30 min | 1 |
| Initial PCR activation | 95° C./31 min | 1 |
| Denaturation | 95° C./32 min | 45 |
| Annealing | 55° C./33 min | |

Instrument: Rotorgene 3000

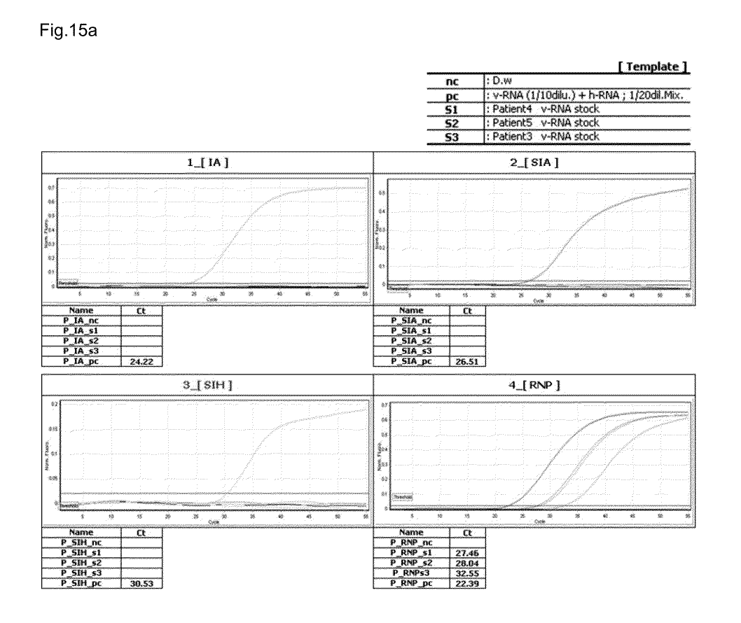

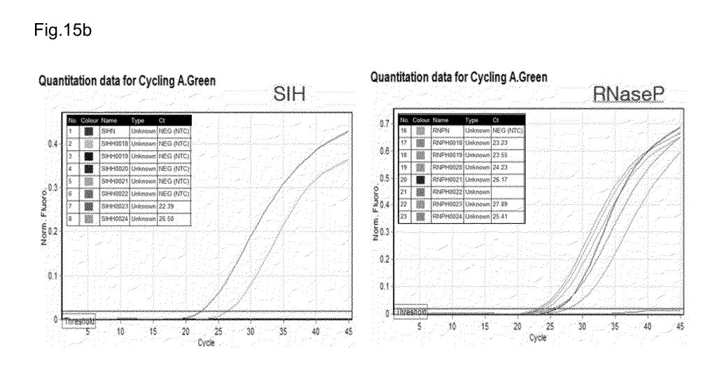

4) After the PCR is completed, perform analysis for each gene and record the result for each sample. FIG. 15 shows the result of real-time RT-PCR.

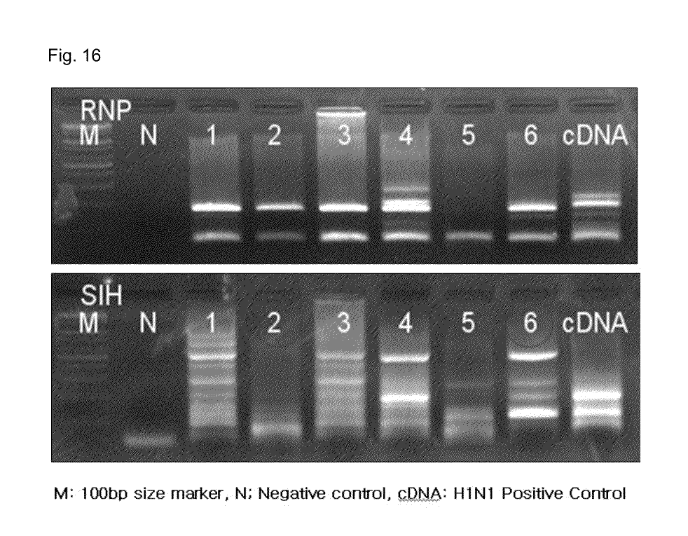

FIG. 16 shows a result of electrophoresing some of the real-time RT-PCR products. As can be seen from the images, test using the DNA chip of the present disclosure or by real-time RT-PCR is necessary for the H1N1 gene since it is difficult to distinguish positive and negative results only with the size of the PCR products.

RT-PCR primers to be used for the chip of the present disclosure were prepared as described in Table 12. One-step RT-PCR was carried out using 0.5 μL of Taq & RT mixture, 12.5 μL of 2×PCR mixture, 1 μL of 10 pmole F & R primers respectively, 5 μL of RNase-free water and 5 μL of viral RNA under the same condition of the real-time RT-PCR as described above.

TABLE 12
PCR primer sequence of influenza A chip

| | | | | | | Size of |
|---|---|---|---|---|---|---|
| | | | | | Size of | PCR |
| | | | Tm | GC | primer | Product |
| No. | Name | Sequence | (° C.) | % | (bp) | (bp) |
| SEQ ID | H1H3F | GGDAATYTAATWGCDCC | 51.34 | 46 | 17 | 226 |
| NO 208 | | | | | | |
| SEQ ID | H1H3R | Cy5-GGKAYRTTTCTYAGDCCTGT | 53.68 | 47 | 20 | |
| NO 209 | | | | | | |
| SEQ ID | N1N2F | ACHCARGAGTCDGAATG | 52.44 | 50 | 17 | 197 |
| NO 210 | | | | | | |
| SEQ ID | N1N2R | Cy3-GAGCCWTKCCARTTRTCYCTGCA | 55.31 | 56 | 23 | |
| NO 211 | | | | | | |

(D stands for G, A or T and H stands for A, C or T.)

10.3. Hybridization and Analysis

On the slide chip on which the oligonucleotide probes were spotted, each 10 μL of the RT-PCR product of the H and N gene were mixed to a final volume of 50 μL as template. After denaturation at 95° C. for 5 minutes, the mixture was immediately transferred to ice and left for 3 minutes. Then, after adding 50 μL of a hybridization solution to make the final volume 100 μL, reaction was performed at 45° C. for 30 minutes with the probes fixed on the slide. The hybridization solution was prepared by mixing 2 mL of 20×SSC with 1.7 mL of 90% glycerol and 6.3 mL of 50 mM phosphate buffer to a final volume of 10 mL. After the hybridization was completed, the well cover was removed from the DNA chip and the chip was immersed in 3×SSPE solution (NaCl (26.295 g), NaH₂PO₄-1H₂O (4.14 g), Na₂EDTA (1.11 g) dissolved in 1 L of distilled water; adjusted to pH 7.4 with 10 N NaOH). After washing at room temperature for 2 minutes and then again with 1×SSPE solution (NaCl (8.765 g), NaH₂PO₄-1H₂O (1.38 g), Na₂EDTA (0.37 g) dissolved in 1 L of distilled water; adjusted to pH 7.4 with 10 N NaOH) at room temperature for 2 minutes, centrifugation was performed at 800 rpm at room temperature for 1.5 minutes followed by drying. After removal of nonspecific signals through washing, the dried slide was subjected to analysis of fluorescence signals and images using a fluorescence scanner. As for the scanner, GenePix 4000B scanner (Axon, USA), ScanArray Lite (Packard Bioscience, USA) or an equipment comparable thereto may be used.

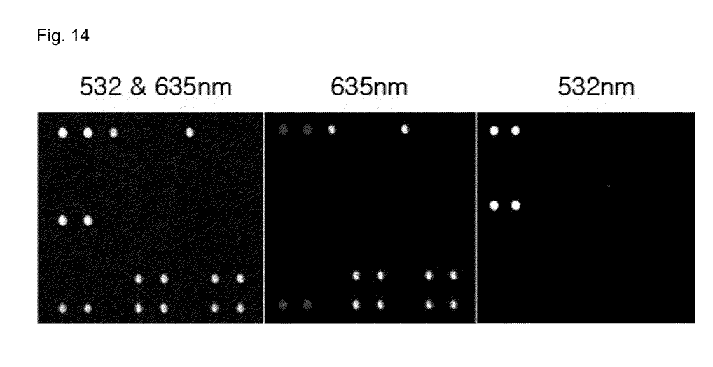

FIG. 13 shows the grids of the influenza virus A DNA chip using the Y-shaped probe described in Table 10, and FIG. 14 shows the result of performing RT-PCR for the control substance and the upper respiratory aspirate sample and hybridizing the product on the influenza A virus DNA chip of the present disclosure. The swine influenza virus A (H1N1)-positive sample was clearly identified. It took about 3-4 hours until the result was obtained according to the method of the present disclosure, and as many as about 800 samples could be tested per day by two researchers using about 100 chips.

The upper respiratory aspirate samples from 783 Korean patients who were suspected of swine influenza virus A (H1N1) infection between November and December of 2009 were tested with the influenza virus genotyping DNA microarray of the present disclosure and with the real-time PCR method recommended by the WHO. As a result, 309 cases (39.5%) were identified as H1N1 influenza virus A/H1N1 infection, and the results of the DNA microarray and the real-time PCR matched 100%.

### Example 11

### Analysis of Gene Expression Using DNA Microarray on which Y-Shaped Probe is Spotted

An important feature of genetic testing is to analyze the transcriptome, i.e. gene expression. Especially, the high-throughput analysis of all the genes expressed in an organism or cell and the expression profile thereof, and the study of the change of the gene expression in response to environment or external stimulation, hormones or drugs, aging, diseases, etc. can be said as the essence of molecular biology. The DNA microarray is a powerful tool for such studies.

Although the early DNA microarray for gene expression studies used complementary DNA (cDNA) or PCR product as probe, various oligonucleotides are used recently. A lot of manufacturers are producing oligonucleotide microarrays capable of studying the expression of all known human genes. Representative products include Affymetrix GeneChip arrays (http://www.affymetrix.com), Multipack gene expression microarrays (Agilent Technology), CodeILink Bioarrays (GE Healthcare/Amersham Bioscience), etc. These products include controls to avoid errors or variations owing to the microarray itself, hybridization reaction or sample and to allow analysis of relative and absolute gene expression levels. Widely used methods are as follows. The first is to spot a probe for the housekeeping gene at the corner of the microarray as internal control or reference. The second method is to include the so-called spike-in RNA or external control RNA as well as the target RNA and carry out hybridization together on the microarray. Through this, the change in relative gene expression can be investigated more accurately and sensitively, and the difference between microarrays can be analyzed more easily. Even the absolute quantity of gene expression can be detectable. However, it is difficult to accurately analyze the variations between the spots and noise or other variables. Indeed, it is reported that the signal intensity of each spot is not proportional to the gene expression level (Yang I V. Use of external controls in microarray experiments. Methods in Enzymology. 2006; 411: 50-63; Salt M. Standards in gene expression experiments. Methods in Enzymology. 2006; 411: 64-80; Irizarry R A, Bolstad B M, Collin F, Cope L M, Hobbs B, Speed T P. Summaries of Affymetrix GeneChip probe level data. Nucleic Acids Res. 2003 Feb. 15; 31(4): e15).

These oligonucleotide microarrays are in common in that the internal control probe is not included in each spot. In contrast, the present disclosure includes both the internal and external controls in the DNA microarray so that more accurate gene expression analysis is possible and standardization can be achieved.

This example describes a new DNA microarray for analyzing gene expression using the Y-shaped probe. The basic concept is as follows. The Y-shaped probe is prepared by including the probe for testing the target gene and the probe for internal reference together. The Y-shaped probe is spotted to fabricate the microarray. Meanwhile, the sample cRNA is fluorescence-labeled and the cRNA of the reference is labeled with another substance. They are mixed and hybridized on the DNA microarray. Thereafter, analysis is performed through normalization by comparing the signal of the sample gene with the fluorescence signal of the reference. The feature of the present disclosure is that, unlike other microarrays, the signals of the target gene and the internal reference are analyzed together for on spot. This means that control experiment is conducted for each spot. This provides many advantages in that error can be minimized when analyzing gene expression using the DNA microarray, more accurate statistical analysis is possible, quality control can be improved, and cost and time can be saved. The present disclosure is expected to lead to advancement in high-throughput transcriptomic studies.

Detailed description will be given hereinafter.

The Y-shaped probe of the present disclosure is prepared by arranging oligonucleotide probes for multiple target genes to be tested for gene expression on one probe part and arranging the oligonucleotide probe for the internal reference gene on the other probe part. Thus prepared multiple Y-shaped probes are spotted on a glass slide to fabricate a microarray. The reference gene is selected such that it lacks complementarity with the target gene and it is not present or expressed in the individual to be tested, for example, human. In this example, a probe for the E. coli motD gene as internal control gene was included in one probe part of the Y-shaped probe.

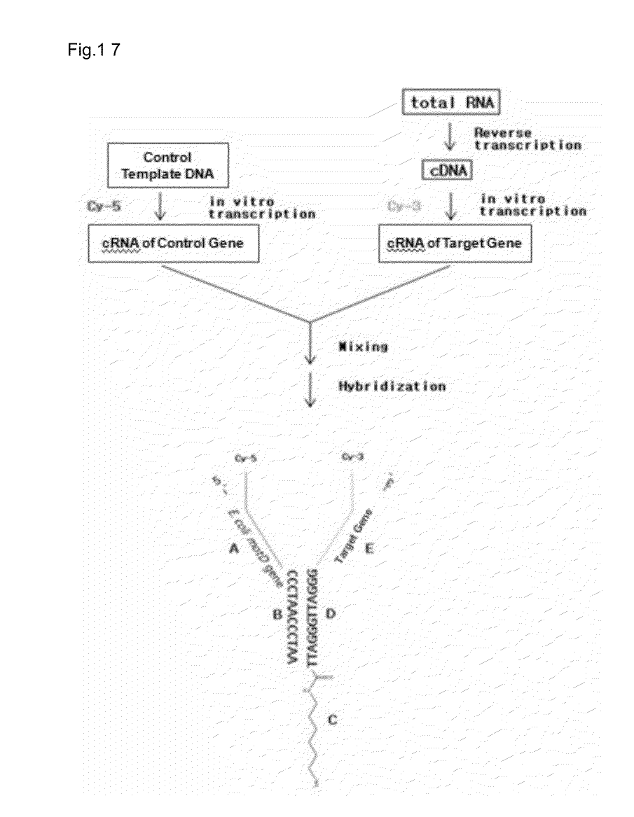

Subsequently, two substances to be placed on the DNA microarray are prepared. Total RNA is isolated from the sample to be tested and cRNA is obtained by in vitro transcription (IVT) and reverse transcription. During this procedure, the cRNA is labeled with a fluorescent dye (e.g., Cy-3). Separately from this, external control is prepared. For this, the control gene, i.e. the E. coli motD gene, is inserted into a plasmid vector comprising the promoter of RNA polymerase (T7, T3, SP6) and a poly-A tail. cRNA is obtained using the vector as template via IVT. Alternatively, the cRNA may be synthesized as oligonucleotide. During the IVT procedure, the cRNA is labeled with a different fluorescent dye (e.g., Cy-5). After confirming the quantity and quality of each cRNA, the cRNAs of the sample to be tested and the control substance are mixed and hybridized on the microarray. Then, analysis is performed using a fluorescence scanner. Through a triple normalization procedure of investigating signals from Cy-5 and Cy-5 after excluding the background noise signal from each spot and comparing with the Cy-3 signal of the housekeeping gene, the ratio of the expression level of the target gene to that of the housekeeping gene can be obtained for each spot. By combining the result, the relative expression level of as many as tens of thousands of genes from the sample can be statistically analyzed. In this manner, high-throughput analysis of the expression of all known human genes is possible (FIG. 17).

In this example, a microarray for analyzing the expression of various genes involved in cellular proliferation was fabricated using the Y-shaped probe. RNA was isolated respectively from human non-small-cell carcinoma tissues, lung tissues of a healthy person and peripheral venous blood leukocytes and expression of signal-transducing genes was analyzed using the microarray of the present disclosure. Also, analysis was performed by the quantitative real-time PCR method for comparison.

As an example, analysis of the expression of the epidermal growth factor receptor (EGFR) genes is described in detail.

11.1. Preparation of Y-Shaped Probe and DNA Microarray

1) Left-Side and Right-Side Probe Parts (A and E in FIG. 1)

A probe for the sense strand of each target gene was arranged on the right-side probe part (E in FIG. 1) of the Y-shaped probe, and a control probe for the E. coli motD gene was arranged on the left-side probe part (A in FIG. 1). Each probe was about 70 bp in length. The probe length may be decreased further, but was selected so to ensure sensitivity.

2) Stem Parts (B and D in FIG. 1)

The left-side stem part (B in FIG. 1) was designed with two CCCTAA which is reverse to the human telomere sequence, and the right-side stem part (D in FIG. 1) was designed with two TTAGGG which is complementary thereto.

3) Linker Part (C in FIG. 1)

The linker part was designed with an internal amino modifier C6 dT (iAmMC6T). The probe was spotted on a glass slide as described above to fabricate a DNA chip. The sequences of the Y-shaped probe for the EGFR gene and the housekeeping gene β-actin are described in Table 13.

TABLE 13
Sequence for Y-shaped probe for analysis of EFGR gene
expression.

| No. | Name | Sequence (5′->3′) | Type |
|---|---|---|---|
| SEQ ID | β-actin | 5′ -GTCGATATCCTGCAACGTTCCGCTGACATCACC | House- |
| NO 212 | | ACCGCCAAATT | keeping |
| | | GCTCCAACACCGTCTCGGCAGCGCT | |
| | | CCCTAACCCTAA -iAmMC6T-TTAGGGTTAGGG | |
| | | CCTGGCACCCAGCACAATGAAGATCAAGATCATTGC | |
| | | TCCTCCTGAGCGCAAGTACTCCGTGTGGATCGGCG | |
| SEQ ID | EGFR | 5′ -GGTCGATATCCTGCAACGTTCCGCTGACATCAC | Target |
| NO 213 | | CACCGCCAAAT | gene |
| | | TGCTCCAACACCGTCTCGGCAGCGCT | |
| | | CCCTAACCCTAA -iAmMC6T-TTAGGGTTAGGG | |
| | | AGCCAGGAACGTACTGGTGAAAACACCGCAGCATG | |
| | | TCAAGATCACAGATTTTGGGCTGGCCAAACTGCTG | |

Note)
Underlined sequence: probe part

11.2. Sample Preparation and Labeling

RNA was isolated according to a known method sample and labeled with Cy-3 through reverse transcription and in vitro transcription (Yu J, Othman M I, Farjo R, Zareparsi S, MacNee S P, Yoshida S, Swaroop A. Evaluation and optimization of procedures for target labeling and hybridization of cDNA microarrays. Mol Vis. 2002 Apr. 26; 8: 130-7; Lonergan W, Whistler T, Vernon S D. Comparison of target labeling methods for use with Affymetrix GeneChips. BMC Biotechnol. 2007 May 18; 7: 24).

Total RNA was isolated from the sample using the Trizol reagent (Invitrogen) and RNeasy kit (Qiagen, Vaklencia, Calif., USA) until the A260/A280 ratio was 1.9 or higher and ribosomal 28S and 18S RNA bands were clearly seen upon electrophoresis. 250 ng of total RNA was mixed with 5.8 μL of T7 promoter primer (Agilent Technologies). After warming at 65° C. for 10 minutes, the mixture was placed on ice. Then, after adding 4.4 μL of cDNA master mix (2 μL 5× first strand buffer, 1 μL of 0.1 M DTT, 0.5 μL of 10 mM dNTP mix, 0.6 μL of Moloney murine leukemia virus reverse transcriptase (MMLV RT) and 0.3 μL RNaseOUT™ (Agilent Technologies) and mixing, the mixture was reacted at 40° C. for 2 hours. Subsequently, after warming at 65° C. for 15 minutes followed by cooling, 0.5 μL of 10 mM Cy™3-CTP, 14.5 μL of transcription master mix (3.83 μL of nuclease-free water, 5 μL of 4× transcription buffer, 2 μL of NTP mix, 1.6 μL of 50% polyethylene glycol (PEG), 0.12 μL of RNaseOUT, 0.15 μL of inorganic pyrophosphatase and 0.3 μL of T7 RNA polymerase) was added and reacted at 40° C. for 2 hours after mixing. Thus formed cRNA of the target gene was purified with the RNeasy kit (Qiagen, Valencia, Calif., USA) and then hybridized on the microarray.

11.3. Preparation and Labeling of External Control Substance

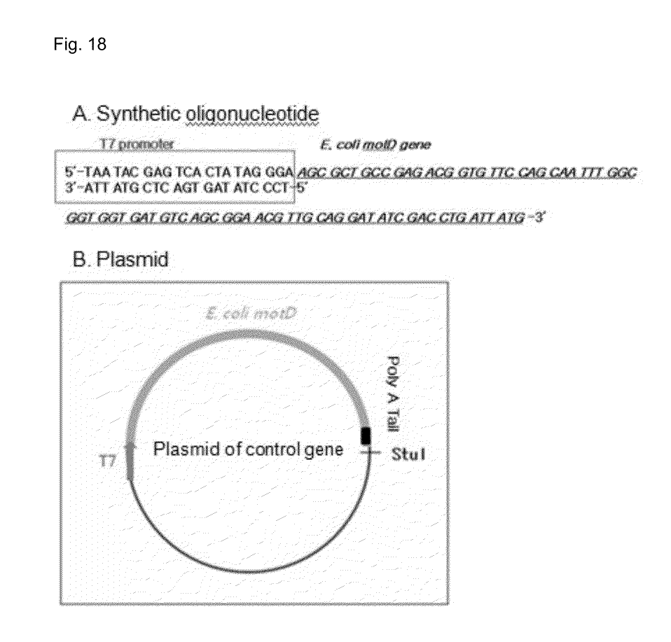

The control gene was labeled by in vitro transcription without reverse transcription. For the control substance, an oligonucleotide comprising the T7 promoter and the E. coli motD gene may be synthesized as shown in FIG. 18A. Alternatively, the E. coli motD gene may be cloned into a plasmid vector comprising the T7 promoter and poly-A tail as shown in FIG. 18B, and it may labeled by in vitro transcription as described above. For the labeling, Cy-5 is used instead of Cy-3.

11.4. Hybridization and Result Analysis

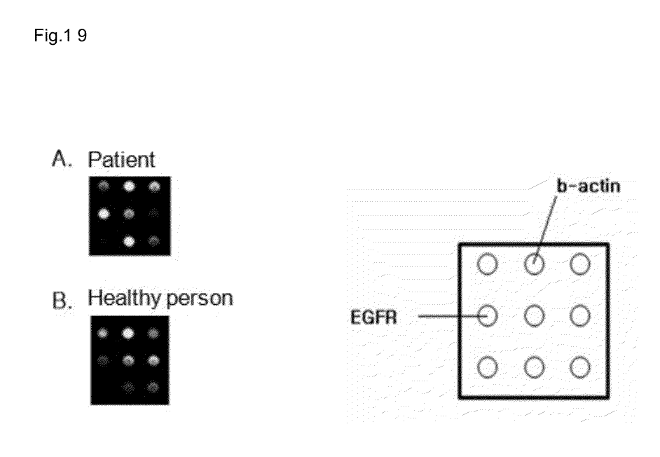

When the target gene and the control gene labeled respectively with Cy-3 and Cy-5 are mixed and hybridized on the microarray, both Cy-3 and Cy-5 signals are emitted from each spot as shown in FIG. 19. In theory, the Cy-5 signal from the control gene should be the same for all the spots. However, since the spots differ in shape and size and in the amount of the probes spotted thereon, the Cy-5 signal from the control gene may vary depending on the spots. Therefore, normalization of the signals is necessary to correct the errors caused by difference in spots. The normalization is achieved by calculating θᵢ (=Rᵢ/Rₘ) by dividing the fluorescence intensity Rᵢ of the control gene from each spot into the mean control gene fluorescence intensity Rₘ (=(SRᵢ)/n) of the total spots of the microarray, and then dividing the fluorescence intensity Sᵢ′ of the target gene of each spot into θᵢ to compute Sᵢ′ (=Sᵢ/θᵢ). By doing so, the error occurring from different spots can be removed. As a result, by comparing the Sᵢ′ value of the target gene with the Sₕₖ′ value of the housekeeping gene, the relative expression level of the target gene (=Sᵢ′/Sₕₖ′) can be computed.

11.5. Comparative Analysis by Real-Time PCR

The relative expression of the EGFR gene with respect to the β-actin gene was investigated by quantitative real-time PCR. After extracting RNA from each sample, cDNA was prepared by reverse transcription. 100 ng of the cDNA was added to a PCR tube and 10 pmol of reverse primer EGFRR or ACTINR for amplifying the EGFR or β-actin gene, 10 pmol of forward primer EGFRF or ACTIN F for amplifying the EGFR or β-actin gene, 12 pmol of EGFRP or ACTINP probe specific for the EGFR or β-actin gene labeled with Cy-3 or Cy-5 respectively, and 25 μL of 2× premix containing PCR buffer (50 mM Tris-HCl, pH 8.3, 250 mM KCl, 7.5 mM MgCl₂), 0.2 I.U. of Taq polymerase and dNTPs were added as described in Table 14. Then, distilled water was added to make the final volume 50 μL. After mixing and centrifugation, followed by warming at 50° C. for 2 minutes and then at 95° C. for 10 minutes using a real-time gene amplifier (Rotor-gene 600), 40 cycles of 15 seconds at 95° C., 20 seconds at 55° C. and 25 seconds at 72° C. were carried out. After the reaction was completed, Ct value was acquired by analyzing the amplification curve. Using the Ct value, the accuracy of relative expression of the EGFR gene with respect to the housekeeping gene was analyzed, and the optimum condition for the Y-shaped probe of the present disclosure was determined.

TABLE 14
Oligonucleotide primers for quantitative real-time PCR

| | | | | Size | TM | GC |
|---|---|---|---|---|---|---|
| No | Gene | Name | Sequence (5′->3′) | (bp) | (° C.) | % |
| SEQ ID | β-actin | ACTINF | AGCCTCGCCTTTGCCGA | 17 | 52 | 65 |
| NO 214 | | | | | | |
| SEQ ID | | ACTINR | CTGGTGCCTGGGGCG | 15 | 53 | 80 |
| NO 215 | | | | | | |
| SEQ ID | | ACTINP | Cy-5-CCGCCGCCCGTCCACACCCGCC-BHQ | 22 | 70 | 86 |
| NO 216 | | | | | | |
| SEQ ID | | EGFRF | CGCAGATAGTCGCCCAAAGTT | 21 | 54 | 52 |
| NO 217 | | | | | | |
| SEQ ID | EGFR | EGFRR | GCATTCTTTCATCCCCCTGAA | 21 | 52 | 48 |
| NO 218 | | | | | | |
| SEQ ID | | EGFRP | Cy-3-CCCGAGACCCCCAGCGCTACC-MGB | 21 | 64 | 76 |
| NO 219 | | | | | | |

(BHQ and MGB are fluorescent labels.)

11.6. Result of Analysis by DNA Microarray and Real-Time PCR

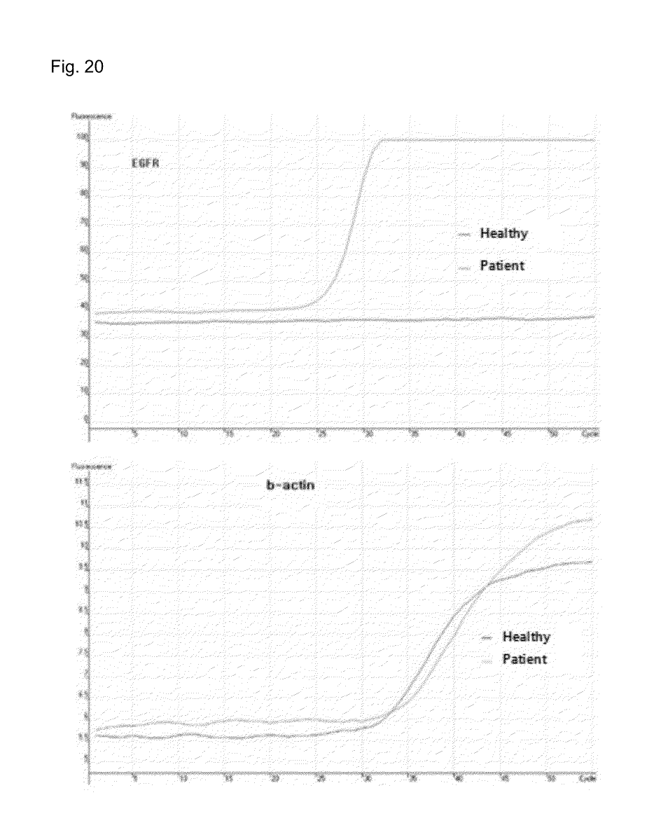

The experimental result is shown in FIGS. 19 and 20. FIG. 19 shows a result of analyzing the expression of the EGFR gene and the β-actin gene using the Y-shaped probe. The Y-shaped probe comprised the housekeeping gene β-actin as target gene and the E. coli motD gene as control gene. The Cy-5 fluorescence intensity (R_{ACTIN}) of the control gene from the spot was divided into the mean Cy-5 fluorescence intensity (Rₘ) of the total spots of the microarray to obtain the value _{ACTIN} (=R_{ACTIN}/Rₘ), and the Cy-3 fluorescence intensity S_{ACTIN} of β-actin was divided into _{ACTIN} to obtain S_{ACTIN}′ (=S_{ACTIN}/_{ACTIN}), which is the normalized expression level of the β-actin gene. In the same way, the normalized expression level of the EGFR gene, S_{EGFR}′ (=S_{EGFR}/_{EGFR}), was calculated using a Y-shaped probe comprising the EGFR as target gene and the E. coli motD gene as control gene. Then, the relative expression of the EGFR gene with respect to the housekeeping gene (=S_{EGFR}′/S_{ACTIN}′) can be determined using the normalized expression level of the β-actin gene (S_{ACTIN}′). The expression level measured in this example using the Y-shaped probe matched with that measured by quantitative real-time PCR as shown in FIG. 20 (R=0.9).

From this result, it can be seen that the Y-shaped probe according to the present disclosure can accurately determine the expression level of a specific gene. The probes for individual genes bind specifically to the RNAs of the corresponding genes in the clinical sample, without cross hybridization between the probes. When the tests were performed 3 or more times with time intervals, the same result was obtained. That is to say, the reproducibility was 100%.

The human lung cancer tissue showed significantly higher expression of the EGFR gene as compared to the normal lung tissue or leukocytes of a healthy person. This suggests that the lung cancer will respond well to the EGFR inhibitors such as gefitinib, erlotinib, lapitinib, cetixiamb, panitumab, etc.

The synthetic oligonucleotide (FIG. 18A) and the plasmid (FIG. 18B) comprising the T7 promoter, poly-A tail and E. coli motD gene used in Example 11 are shown in FIG. 18. They were used as templates to label the target with Cy-5 by in vitro transcription. Then, after mixing with the cRNA obtained from the sample, hybridization was performed on the DNA microarray. FIG. 19 shows a result of extracting RNA from the samples of a healthy person and a patient, synthesizing cDNA therefrom, and analyzing expression of the EGFR gene and the β-actin gene using the Y-shaped probe microarray.

### Example 12

### SNP Analysis Using DNA Microarray on which Y-Shaped Probe is Spotted

The most technically difficult thing in genotyping is to analyze genetic variation in single nucleotide level. In particular, development of a method for high-throughput analysis of multiple genes accurately and quickly at minimum cost is very important.

Methods allowing high-throughput analysis of the genetic variation in single nucleotide sequence include (1) allele-specific hybridization (ASH), (2) flap endonuclease discrimination, (3) primer extension, (4) allele-specific digestion and (5) oligonucleotide ligation (OLA). These methods analyze the nucleotide sequence by labeling the reaction product with fluorophore or biotin. Appliedc Biosystem's microplate reader and capillary electrophoresis system, Sequenom's mass spectrometer, Pyrosequencing AB's CCD camera, Luminex's microbead, and DNA microarray are used for the analysis. Recently, the DNA microarray is the most widely used and it is also used to analyze single nucleotide polymorphism of the entire human genome (Tsuchihashi Z and Dracopoli N C. Progress in high throughput SNP genotyping methods. The Pharamacogenomics Journal. 2002; 2: 103-110; Jenkins S and Gibson N. High-throughput SNP genotyping. Comparative and Functional Genomics. 2002; 3: 57-66).

In Example 12, a method for analyzing SNP by allele-specific hybridization on a DNA microarray on which the Y-shaped probe is spotted and applying it for clinical practice is described.

Although SNP is also a genetic variation in nucleotide sequence, it is distinctly different from mutation. SNP is a frequent variation occurring with a frequency of 1% or higher in humans, altering physique, appearance, character, disease risk and drug response between individuals. Although SNP itself does not cause diseases, it increases or decreases the risk of a particular disease through interaction with other genes or environmental factors such as diet and lifestyle. In contrast, mutation is rarer with a frequency of less than 1% in humans. It denatures proteins and may cause diseases as it is. Mutation is frequently a pathogenic variation, causing inherited genetic diseases or acquired diseases. A typical example of the latter is cancer. Cancer occurs as a result of accumulation of mutations of oncogenes or tumor suppressor genes. Accordingly, SNP analysis tends to be helpful in disease prediction, and mutation analysis in disease diagnosis.

The Y-shaped probe of the present disclosure or a variant thereof may be used to test SNP on the DNA microarray by allele-specific hybridization as follows.

First, a d-shaped probe which is a variant of the Y-shaped probe may be used. For example, a d-shaped probe with a probe for the SNP site of the target gene arranged on the right-side probe part of the Y-shaped probe and the left-side probe part removed is used to fabricate the microarray. Allele-specific probes are prepared for the wild type and mutant type, and the nucleotide differing in the two probes is located at the center of the probes. The probes may be about 15-30 bp in length. The target genes are labeled identically with Cy-3 or Cy-5, and the probes of the spot exhibiting perfect match after hybridization are found. In this manner, it can be identified whether the target gene is wild type or mutant type. Analysis is possible with a single-color fluorescence scanner.

Second, a Y-shaped probe is prepared by arranging a probe for the SNP site of the sense strand of the target gene to be detected on the right-side probe part of the Y-shaped probe and arranging a control probe lacking the SNP site of the antisense strand of the target gene on the left-side probe part as internal reference, and a microarray is fabricated using the same. Then, when PCR is performed after labeling the sense strand for SNP analysis and the antisense strand for the control gene with different fluorophores, e.g. Cy-3 and Cy-5, the SNP site of the target gene is amplified with the Cy-3 label and the antisense strand is amplified with the Cy-5 label. If the product hybridized on the microarray after being placed thereon as a single strand, the amplification product of the sense strand gene exhibits Cy-3 signal and the amplification product of the antisense strand gene exhibits Cy-5 signal.

The Cy-5 signal is an internal reference signal and the Cy-3 signal is the SNP test signal. By normalizing the Cy-3 signal with respect to the Cy-5 signal after removing the background signal from each spot, as described in Example 11, the perfectly matching probe is found. For this, a dual-color fluorescence scanner is necessary.

This examples describes use of the latter of the two, i.e. the Y-shaped probe. For this, a DNA microarray for analyzing SNP of genes related with various aging-associated diseases, especially heart disease, dementia and age-related macular degeneration (AMD), was prepared. Use of the former, i.e. the d-shaped probe, will be described in detail in Example 13.

The DNA microarray for SNP detection according to the present disclosure may be used to predict the risk of important adult diseases and to prevent them.

12.1. Preparation of Y-Shaped Probe

A Y-shaped probe for multiple genes including Alzheimer's disease-associated gene (apolipoprotein E; Apo E), interleukin 1A (IL1A), angiotensin-converting enzyme (ACE), nitric oxide synthase 3 (NOS3), estrogen receptor alpha (ESR1), methylenetetrahydrofolate reductase (MTHFR), β-2 adrenergic receptor (ADRB2), cholesterylester transfer protein (CETP) and complement factor H (CFH) was designed as follows according to the design method of the Y-shaped probe of the present disclosure, based on the known nucleotide sequence (NCBI dbGAP SNP) was used.

1) Left-Side and Right-Side Probe Parts (A and E in FIG. 1)

A probe for the SNP site of the sense strand of each target gene was arranged on the right-side probe part (E in FIG. 1) of the Y-shaped probe. Allele-specific probes were prepared for the wild type and mutant type, and the nucleotide differing in the two probes was located at the center of the probes. The probes were 15-28 bp in length. A control probe lacking the SNP site of the antisense strand of the target gene was used as the right-side probe part.

2) Stem Parts (B and D in FIG. 1)

The left-side stem part (B in FIG. 1) was designed with two CCCTAA which is reverse to the human telomere sequence, and the right-side stem part (D in FIG. 1) was designed with two TTAGGG which is complementary thereto.

3) Linker Part (C in FIG. 1)

The linker part was designed with an internal amino modifier C6 dT (iAmMC6T). A total of 96 Y-shaped probes for SNP analysis of aging-associated diseases were designed. The probe was spotted on a glass slide as described above to fabricate a DNA chip. The name, SEQ ID NO and genotype of some representative probes are summarized in Table 15.

TABLE 15
Sequence of Y-shaped probe for SNP detection

| No. | □□ | | □ | □□□ (5′->3′) |
|---|---|---|---|---|
| SEQ ID | ACE | Ins > | Wild | GCCTTGAGCTCCAGCCCTTAGCCCCTAACCCTAA-i |
| NO 220 | | Del | | AmMC6T-TTAGGGTTAGGGCCAAAGTGCTGGGATT |
| | | Intron | | ACAGGCGTGATA |
| SEQ ID | ACE | 16 | Mutant | GCCTTGAGCTCCAGCCCTTAGCCCCTAACCCTAA-i |
| NO 221 | | | | AmMC6T-TTAGGGTTAGGGGACCTGCTGCCTATAC |
| | | | | AGTCACTTTT |
| SEQ ID | ADRB2 | C->G | Wild | GGACGATGAGAGACATGACGACCCTAACCCTAA-iA |
| NO 222 | | | | mMC6T-TTAGGGTTAGGGTCACGCAGCAAAGGGAC |
| SEQ ID | ADRB2 | (Gln | Mutant | GGACGATGAGAGACATGACGACCCTAACCCTAA-iA |
| NO 223 | | 27Glu) | | mMC6T-TTAGGGTTAGGGTCACGCAGGAAAGGGAC |
| SEQ ID | Apo E | T->C | Wild | AGGTGGGAGGCGAGGCCCTAACCCTAA-iAmMC6T- |
| NO 224 | | | | TTAGGGTTAGGGAGGACGTGTGCGGCCGC |
| SEQ ID | | | Mutant | AGGTGGGAGGCGAGGCCCTAACCCTAA-iAmMC6T- |
| NO 225 | | | | TTAGGGTTAGGGGAGGACGTGCGCGGCCG |
| SEQ ID | Apo E | C->T | Wild | AGGTGGGAGGCGAGGCCCTAACCCTAA-iAmMC6T- |
| NO 226 | | | | TTAGGGTTAGGGTGCAGAAGCGCCTGGCA |
| SEQ ID | | | Mutant | AGGTGGGAGGCGAGGCCCTAACCCTAA-iAmMC6T- |
| NO 227 | | | | TTAGGGTTAGGGTGCAGAAGTGCCTGGCA |
| SEQ ID | CETP | Taq1 | Wild | CTATACCTGGCTGTTTGCCCCTAACCCTAA-iAmMC6 |
| NO 228 | | B | | T-TTAGGGTTAGGGTGGGGTTCGAGTTAGGG |
| SEQ ID | CETP | g->a | Mutant | CTATACCTGGCTGTTTGCCCCTAACCCTAA-iAmMC6 |
| NO 229 | | G15 | | T-TTAGGGTTAGGGTGGGGTTCAAGTTAGGG |
| | | 33A | | |
| SEQ ID | CFH | Y40 | Wild | ACGTCTATAGATTTACCCCCCTAACCCTAA-iAmMC6 |
| NO 230 | | 2H | | T-TTAGGGTTAGGGCAAAATCATGGAAGAAA |
| SEQ ID | CFH | (127 | Mutant | ACGTCTATAGATTTACCCCCCTAACCCTAA-iAmMC6 |
| NO 231 | | 7 T- | | T-TTAGGGTTAGGGCAAAATCACGGAAGAAA |
| | | C) | | |
| SEQ ID | ESR1 | IVS | Wild | ACATACTACCTGCACCAGAACCCTAACCCTAA-iAm |
| NO 232 | | Pos. | | MC6T-TTAGGGTTAGGGGTCCCAGCTGTTTTATG |
| SEQ ID | ESR1 | −401 | Mutant | ACATACTACCTGCACCAGAACCCTAACCCTAA-iAm |
| NO 233 | | T > C | | MC6T-TTAGGGTTAGGGGTCCCAGCCGTTTTATG |
| | | (p > P) | | |
| SEQ ID | IL1A | +484 | Wild | CCCTCAATCAAAGTATAACCCTAACCCTAA-iAmMC6 |
| NO 234 | | 5, | | T-TTAGGGTTAGGGAAAAGGTGCTGACCTAG |
| SEQ ID | IL1A | G/T | Mutant | CCCTCAATCAAAGTATAACCCTAACCCTAA-iAmMC6 |
| NO 235 | | | | T-TTAGGGTTAGGGAAAAGGTGATGACCTAG |
| SEQ ID | MTHFR | 677 | Wild | TCACCTGGATGGGAAAGACCCTAACCCTAA-iAmMC |
| NO 236 | | c->t | | 6T-TTAGGGTTAGGGTGCGGGAGCCGATTTCA |
| SEQ ID | MTHFR | | Mutant | TCACCTGGATGGGAAAGACCCTAACCCTAA-iAmMC |
| NO 237 | | | | 6T-TTAGGGTTAGGGTGCGGGAGTCGATTTCA |
| SEQ ID | NOS3 | T > C, | Wild | TTTCCTGCCCTCTCTTTCCTCCCTAACCCTAA-iAmM |
| NO 238 | | −786 | | C6T-TTAGGGTTAGGGTGAGGCTGGTGACTAAA |
| SEQ ID | NOS3 | pro- | Mutant | TTTCCTGCCCTCTCTTTCCTCCCTAACCCTAA-iAmM |
| NO 239 | | moter | | C6T-TTAGGGTTAGGGTGAGGCTGTTGACTAAA |

Note)
Underlined sequence: probe part

12.2. PCR

After isolating DNA from each sample, PCR was performed while adding a fluorescent dye. The sense strand for SNP analysis was labeled with Cy-3, and the antisense strand for the control gene was labeled with Cy-5. After PCR, the SNP site of the target gene is amplified as labeled with Cy-3 and the control gene of the antisense strand is amplified as labeled with Cy-5. The sequences of PCR primers are summarized in Table 16. PCR was carried out for 35 cycles after initial denaturation at 96° C. for 3 minutes. Each cycle consisted of 30 seconds at 94° C., 30 seconds at 58° C. and 30 seconds at 72° C. Final extension was carried out at 72° C. for 5 minutes.

TABLE 16
Sequence of primers for SNP analysis

| No. | □□ | □ | □□□ (5′->3′) |
|---|---|---|---|
| SEQ ID | ACE, | Reporter | 5′ -Cy-3- CTG GAG AGC CAC TCCCAT CCT |
| NO 240 | forward | | TTC T |
| SEQ ID | ACE, | Reference | 5′ -Cy-5- GAC GTG GCC ATCACA TTC GTC |
| NO 241 | reverse | | AGA T |
| SEQ ID | ADRB2, | Reporter | 5′ -Cy-3- CTT CTT GCT GGC ACC CAA T |
| NO 242 | Forward | | |
| SEQ ID | ADRB2, | Reference | 5′ -Cy-5-CAG GCC AGT GAA GTG ATG AA |
| NO 243 | Reverse | | |
| SEQ ID | Apo E, | Reporter | 5′ -Cy-3-AAT CGG AAC TGG AGG AAC AAC |
| NO 244 | Forward | | |
| SEQ ID | Apo E, | Reference | 5′ -Cy-5-GGC CTG GTA CAC TGC CA |
| NO 245 | Reverse | | |
| SEQ ID | CETP, | Reporter | 5′ -Cy-3-CTC GCC TTC AAG GTC AAG T |
| NO 246 | Forward | | |
| SEQ ID | CETP, | Reference | 5′ -Cy-5-TGG CTC AGA TCT GAA CCC TA |
| NO 247 | Reverse | | |
| SEQ ID | CFH, | Reporter | 5′ -Cy-3-TCATTGTTATGGTCCTTAGGAAA |
| NO 248 | Forward | | |
| SEQ ID | CFH, | Reference | 5′ -Cy-5-ACTGTGGTCTGCGCTTTTG |
| NO 249 | Reverse | | |
| SEQ ID | ESR1, | Reporter | 5′ -Cy-3- ATC CAG GGT TAT GTG GCA AT |
| NO 250 | Forward | | |
| SEQ ID | ESR1, | Reference | 5′ -Cy-5-TCC TTG GCA GAT TCC ATA GC |
| NO 251 | Reverse | | |
| SEQ ID | IL1A, | Reporter | 5′ -Cy-3- AAT GAA AGG AGG GGA |
| NO 252 | Forward | | GGATGA CAG AAA TGT |
| SEQ ID | IL1A, | Reference | 5′ -Cy-5- ATG GTT TTA GAA ATC ATC |
| NO 253 | Reverse | | AAGCCT AGG TCA |
| SEQ ID | MTHFR, | Reporter | 5′ -Cy-3-AGG ACT CTC TCT GCC CAG TC |
| NO 254 | Forward | | |
| SEQ ID | MTHFR, | Reference | 5′ -Cy-5- GGA AGA ACT CAG CGA ACT CA |
| NO 255 | Reverse | | |
| SEQ ID | NOS3, | Reporter | 5′ -Cy-3- CCC CTG AGT CAT CTA AGT ATT |
| NO 256 | Forward | | C |
| SEQ ID | NOS3, | Reference | 5′ -Cy-5- AGC TCT GGC ACA GTC AAG |
| NO 257 | Reverse | | |

12.3. Hybridization and Analysis

The PCR products labeled with Cy-3 and Cy-5 were mixed with a hybridization buffer and then hybridized on the microarray at 42° C. for 1 hour. After washing and drying, the result was analyzed using a dual-color fluorescence scanner. Cy-3 is excited at 550 nm and emits signal at 570 nm, whereas Cy-5 is excited at 649 nm and emits signal at 670 nm. The PCR products were also analyzed according to a known nucleotide sequencing method for comparison as described above. By normalizing the Cy-3 signal with respect to the Cy-5 signal after removing the background signal from each spot, the perfectly matching probe is found. By doing so, it can be identified whether the target gene is wild type or mutant type as well as its heterozygosity.

The PCR products were also analyzed according to a known nucleotide sequencing method for comparison. The result of the DNA microarray test was consistent with that of the sequencing analysis in all of 96 cases.

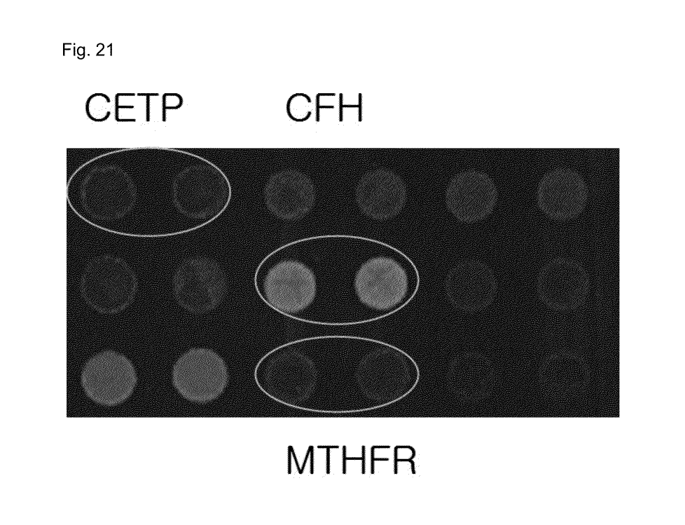

FIG. 21 shows a result of the DNA microarray test for a sample from obese middle-aged male who had been smoking for 25 years. Unfavorable (high-risk) SNP was found in the CFH, CETP and MTHFR genes. The result can be interpreted as follows.

SNP was found at the 402nd codon of the CFH gene (Y402H, rs1061170). CFH plays a key role in immune and inflammatory responses. When there is SNP in the CFH gene, the risk of age-related macular degeneration (AMD) is 2.4-6.3 times higher. Age-related macular degeneration is a major cause of loss of vision in older adults, with over 10 million patients worldwide. The risk increases to about 20 times in those who smoke. Accordingly, for prevention of the disease, quitting smoking, wearing sunglasses on sunny days, eating a lot of vegetables having antioxidative functions, and taking nutrients such as lutein, zeaxanthine and asaxanthane are recommended (Schnoll H P N, Fleckenstein M, Issa P C, Keilhauer C, Holtz F G, Weber B H F. An update on the genetics of aging-related macular degeneration. Molecular Vision. 2007; 13: 196-205).

Also, SNP was found at the 1553rd nucleotide of the CETP gene (G1533A). CETP is an enzyme that facilitates the transfer of triglycerides and cholesteryl esters between high-density lipoproteins (HDL) and low-density lipoproteins (LDL). Unfavorable SNP in the CETP gene increases its activity, thereby increasing serum LDL level and lowering HDL level. As a result, the risk of hyperlipidemia and cardiovascular diseases increases. For prevention of the diseases, the followings are recommended: reduced intake of trans fat and fast food, balanced intake of omega-3 and omega-6 fatty acids, periodic testing of blood LDL level, and intake of CETP-reducing drug when the level is high (Vincent S, Planells R, Defoort C, Bernard M C, Gerber M, Prudhomme J, Vague P, Lairon D. Genetic polymorphisms and lipoprotein responses to diets. Proc Nutr Soc. 2002; 61(4): 427-34).

And, SNP was found at the 677th nucleotide of the MTHFR gene (C677T, Ala222Val). MTHFR is an enzyme playing a key role in the metabolism of homocysteine and folic acid. Unfavorable SNP in the MTHFR gene results in decreased activity of MTHFR and accumulation of homocysteine in the body, leading to arteriosclerosis and increasing the risk of myocardial infarction, dementia, or the like. Especially, the risk is even higher for smokers and when unfavorable SNP is also found in the CETP. In this case, sufficient and consistent intake of four types of vitamin B, i.e. vitamin B₁₂, vitamin B₆, riboflavin and folic acid is recommended, and quitting smoking is necessary (Trabetti E. Homocysteine, MTHFR gene polymorphisms, and cardio-cerebrovascular risk. J Appl Genet. 2008; 49(3): 267-82).

### Example 13

### Screening of Oncogene Mutation Using DNA Microarray

In this example, use of the DNA microarray on which the variant of the Y-shaped probe for analysis of mutation by allele-specific hybridization (ASH) and application thereof to clinical practice are described.

Mutation of genes may cause diseases by inducing change in proteins. About half of human diseases are directly or indirectly caused by genetic mutation. Further, the characteristics of diseases and response to treatment may vary according to the pattern of mutation. This is even more so for cancer. Thus, screening of the mutation of oncogenes or tumor suppressor genes is of great help in diagnosis and early detection of cancer, prognosis evaluation, determination of therapeutic regimen and selection of treatment drug. K-RAS is a representative example.

K-RAS is the most representative oncogene in human. K-RAS plays critical roles in signal transduction for cellular proliferation along with BRAF, EGFR and its subtypes HER-2/erbB2, HER-3 and HER-4. Indeed, more than half of all human cancers, especially adenocarcinoma, are associated with abnormalities of them. The abnormality of K-RAS is mainly caused by point mutation, which constantly activates (turn on) K-RAS, causing hyperproliferation and canceration due to uncontrolled signaling. The point mutation of K-RAS occurs mainly at codons 12 and 13. In particular, mutation at codon 12 accounts for 90%. Although infrequent, mutation also occurs at codons 59 and 61 (Stahel R A. Adenocarcinoma, a molecular perspective. Annals of Oncology. 2007; 18 (supplement 9): 147-149).

The mutation of K-RAS is found in about 20% of all human cancers. It occurs the most frequently in pancreatic cancer (90%), followed by colon cancer (50%) and lung cancer, especially adenocarcinoma, (50%). Accordingly, attempts are made to investigate K-RAS mutation from pancreatic juice, feces, blood, phlegm, etc. in order to diagnose pancreatic cancer, colorectal cancer, lung cancer, or the like. This allows the detection of early cancer not identifiable with the radiological examination and is expected to significantly improve the cure rate of cancer (Kondo H, Sugano K, Fukayama N, Kyogoku A, Nose H, Shimada K. Detection of point mutations in the K-ras oncogene at codon 12 in pure pancreatic juice for diagnosis of pancreatic carcinoma. Cancer 1994; 73: 1589-1594; Prix L, Uciechowski P, Bockmann B, Giesing M and Schuetz A J. Diagnostic biochip array for fast and sensitive detection of K-ras mutations in stool. Clinical Chemistry. 2002; 48(3): 428-435; Hibi K, Robinson C R, Booker S, Wu L, Hamilton S R, Sidransky D, Jen J. Molecular detection of genetic alterations in the serum of colorectal cancer patients. Cancer Research. 1998; 58: 1405-1407).

The cancer associated with K-RAS mutation is distinguished from other cancers in progress or prognosis. Cancers with K-RAS mutation tend to show worse prognosis, relatively higher relapse after surgery, and shorter survival time (Cerottini J P, Caplin S, Saraga E, Givel J C, Benhattar J. The type of K-ras mutation determines prognosis in colorectal cancer. American Journal of Surgery. 1998; 175: 198-202). For this reason, more caution is necessary after the surgery and effective anticancer agent needs to be used upon relapse. However, cancers associated with K-RAS mutation often show resistance to anticancer agents.

Presently, three types of anticancer agents are used. The first is the anticancer agent in traditional sense. To be precise, they are cytotoxic chemotherapy drugs killing not only the cancer cells but also normal cells. Accordingly, their side effect is often a concern. Recently, targeted therapy drugs that attack and destroy only the specific targets of cancer cells are used. They include antibodies, particularly monoclonal antibodies, and synthetic drugs. Another type is the drugs that attack not the cancer but the blood vessels or other accessory tissues of the cancer. Recently, the targeted therapy is attempted actively and combination of two or more types is also attempted widely. For adenocarcinoma in particular, antibody drugs or (cetuximab or panitimab) synthetic drugs (erlotinib, gefitinib or lapitinib) targeting EGFR-HER-2 are expected as a new standard therapy. Lung cancer or colon cancer associated with K-RAS mutation is resistant to most cytotoxic chemotherapy drugs. What is unfortunate is that the cancer associated with K-RAS mutation is also resistant to the targeted therapy drugs. For this reason, development of a new type of an anticancer drug targeting mutant K-RAS, particularly a genetic drug, is imminent (Linardou H, Dahabreh I J, Kanaloupiti D, Siannis F, Bafaloukos D, Kosmidis P, Papadimitriou C A, Murray S. Assessment of somatic k-RAS mutations as a mechanism associated with resistance to EGFR-targeted agents: a systematic review and meta-analysis of studies in advanced non-small-cell lung cancer and metastatic colorectal cancer. Lancet Oncology. 2008; 9(10): 962-72; Bepler G, Begum M, Simon G R. Cancer Control. Molecular analysis-based treatment strategies for non-small cell lung cancer. 2008 April; 15(2): 130-9).

According to the above literatures, it can be seen that the development of a DNA microarray allowing high-throughput detection of K-RAS mutation accurately and quickly at low cost is important. In this example, a DNA microarray on which the Y-shaped probe of the present disclosure is spotted was used to analyze the mutation of the K-RAS gene.

A d-shaped probe with a probe for detecting the mutation of the target gene arranged on the right-side probe part of the Y-shaped probe and the left-side probe part removed was used (FIG. 22). Probes specific for each base A, C, G and T are arranged on the right-side probe part. The nucleotide of the mutation site is located at the center of the probe. The probe is 15-30 bp in length. The probe is spotted to fabricate a microarray. After isolating DNA from a sample, PCR is performed while labeling the target gene K-RAS with Cy-3 or Cy-5. The PCR product is hybridized on the microarray, and the probes of the spot exhibiting perfect match are found by analyzing the fluorescence signal using a scanner. In this manner, it can be identified whether the nucleotide sequence of the target gene is A, C, G or T, i.e. whether it is wild type or mutant type.

Since the DNA microarray of the present disclosure allows accurate detection of the mutation of the K-RAS gene, it may be used to diagnose lung cancer, pancreatic cancer or colon cancer, predict prognosis in cancer patients, and avoid EGFR blocking drugs or antibody drugs to which the mutation is resistant. Accordingly, the DNA microarray of the present disclosure is helpful in diagnosis of cancer, evaluation of prognosis and determination of therapeutic regime.

13.1. Preparation of Probe and DNA Microarray

The d-shaped probe of the present disclosure was described as described in Table 17. One wild-type and six mutant-type probes were prepared for codon 12, and one additional positive control probe was prepared.

TABLE 17
Sequence of d-type probe for condon 12 of K-RAS

| | | | | Reference |
|---|---|---|---|---|
| | Codon and | | | (ATCC |
| No. | amino acid | sequence (5′->3′) | Type | No) |
| SEQ ID | K-ras 12 GGT (Gly) | CCCTAACCCTAA-iAmMC6T-TTA | Wild | LNCaP |
| NO 258 | | GGGTTAGGGGGAGCTGGTGG | | |
| | | CGTA | | |
| SEQ ID | K-ras 12 AGT (Ser) | CCCTAACCCTAA-iAmMC6T-TTA | Mutant | A549 |
| NO 259 | | GGGTTAGGGGGAGCTAGTGG | | |
| | | CGTA | | |
| SEQ ID | K-ras 12 CGT (Arg) | CCCTAACCCTAA-iAmMC6T-TTA | Mutant | H157 |
| NO 260 | | GGGTTAGGGGGAGCTCGTGG | | |
| | | CGTA | | |
| SEQ ID | K-ras 12 TGT (Asp) | CCCTAACCCTAA-iAmMC6T-TTA | Mutant | Mia-PaCa- |
| NO 261 | | GGGTTAGGGGGAGCTTGTGG | | 2 |
| | | CGTA | | |
| SEQ ID | K-ras 12 GAT (Asp) | CCCTAACCCTAA-iAmMC6T-TTA | Mutant | LS174T |
| NO 262 | | GGGTTAGGGGGAGCTGATGG | | |
| | | CGTA | | |
| SEQ ID | K-ras 12 GCT (Ala) | CCCTAACCCTAA-iAmMC6T-TTA | Mutant | SW1116 |
| NO 263 | | GGGTTAGGGTGGAGCTGCTG | | |
| | | GCGTA | | |
| SEQ ID | K-ras 12 GTT (Val) | CCCTAACCCTAA-iAmMC6T-TTA | Mutant | SW620 |
| NO 264 | | GGGTTAGGGGGAGCTGTTGG | | |
| | | CGTA | | |
| SEQ ID | K-ras P/C | CCCTAACCCTAA-iAmMC6T-TTA | Positive | LNCaP |
| NO 265 | | GGGTTAGGGGCCTTGACGATA | control | |
| | | CAGCTA | | |

Note)
Underlined sequence: probe part

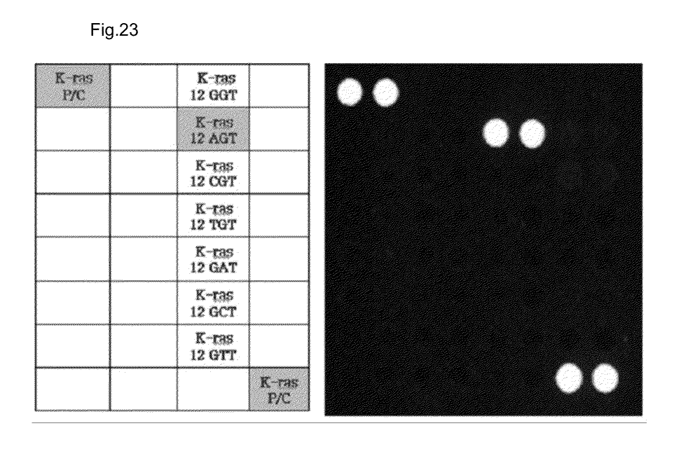

FIG. 23 shows grids of the K-RAS DNA microarray. As seen from Table 17, since the positive control (P/C) was designed with codons 18-23 avoiding codons 12, 13, 59 and 61 where mutation occurs in the cDNA of K-RAS, the K-RAS is detectable without regard to mutation. That is to say, the positive control probe serves as a kind of corner marker.

13.2. Sample Preparation and DNA Isolation

Human cancer cells with known K-RAS mutation profiles were purchased from the American Type Culture Collection (ATCC) and used as reference sample. Details are described in Table 17. Further paraffin-embedded tissues and 20 mL of peripheral venous blood were obtained respectively from 10 lung cancer patients, 10 colon cancer patients and 3 pancreatic cancer patients. Then, cancer cells were isolated from the former by microdissection and serum was isolated from the latter. Subsequently, DNA was isolated from each sample according to a known method (Gilje B, Heikkila R, Oltedal S, Tjensvoll K, Nordgard O. High-fidelity DNA polymerase enhances the sensitivity of a peptide nucleic acid clamp PCR assay for K-ras mutations. Journal of Molecular Diagnosis. 2008. 10(4): 325-31).

13.3. PCR

PCR was performed after adding sterilized triply distilled water, sample DNA, and primers of K-RAS (forward primer: 5′-GACTGAATATAAACTTGTGG-3′, reverse primer: 5′-Cy-5-CTATTGTTGGATCATATTCG-3′) into a tube. The PCR was carried out as described in Table 18 by adding the PCR mixture to a 0.2-mL PCR tube.

TABLE 18
PCR condition for K-RAS DNA microarray
Composition and content of PCR mixture

| | |
|---|---|
| Distilled water | 21.8 μl |
| 10× buffer | 3 μl |
| 2.5 mM dNTP | 2 μl |
| K-RAS primer, forward/reverse mixture (10 pmole/μl) | 1 μl each |
| BioPol Taq (5 U/μl)) | 0.2 μl |
| Template DNA (>150 ng)* | 1 μl |
| Final volume | 30 μl |

PCR condition

| | | |
|---|---|---|
| Predenaturation | 94° C./5 min | 1 Cycle |
| Denaturation | 94° C./30 sec | 35 Cycles |
| Annealing | 55° C./30 sec | |
| Extention | 72° C./30 sec | |
| Final Extention | 72° C./7 min | 1 Cycle |

13.4. Hybridization and Analysis

The PCR product was hybridized on the microarray and analyzed using a scanner. Also, nucleotide sequencing of the PCR product was carried out according to a known method for comparison.

The DNA microarray of the present disclosure could accurately genotype the codon 12 of K-RAS in all the references. K-RAS mutation was found in 11 out of the 23 paraffin-embedded tissues. The result of sequencing was consistent with that of the microarray. Upon blood sample test, K-RAS mutation was identified in 10 out of the 11 cancer tissues by the DNA microarray and in 8 cases by the sequencing analysis. FIG. 23 shows the result of analyzing the blood sample of a lung cancer patient using the K-RAS DNA microarray. Mutation of the codon 12 of K-RAS from GTT to AGT (Gly 12 Ser) was identified, which was also confirmed by the sequencing analysis.

### Example 14

### Screening of Oncogene Mutation Using DNA Microarray

This example also describes analysis of K-RAS mutation on the DNA microarray by ASH, but with different probe structure and analysis method.

A probe for detecting the mutation site of the target gene in forward direction is arranged on the right-side probe part of the Y-shaped probe, and an internal control probe for the antisense strand of the target gene lacking mutation is arranged on the left-side probe part. Probes specific for each base A, C, G and T are arranged on the right-side probe part. The nucleotide of the mutation site is located at the center of the probe. The probe is 15-25 bp in length. The probe is spotted to fabricate a microarray. After isolating DNA from a sample, PCR is performed while labeling the forward strand for testing the mutation of the target gene K-RAS with Cy-3 and labeling the control gene sequence of the other strand with a different fluorophore such as Cy-5. The PCR product is hybridized on the microarray, and the fluorescence signal is analyzed using a scanner. The signals of the control probe and the test probe are normalized as described in the foregoing examples.

14.1. Preparation of Probe and DNA Microarray

The Y-shaped probe of the present disclosure was prepared as described in Table 19. Probes were prepared for one wild type and six mutant types for the codon 12. Separately from these, an additional positive control probe was prepared.

TABLE 19
Sequence of Y-type probe for codon 12 of K-RAS

| | | | | Reference |
|---|---|---|---|---|
| | Codon and | | | (ATCC |
| No. | amino acid | Sequence | Type | No.) |
| SEQ ID | K-ras 12 | TAGCTGTATCGTCAAGGCCCC | Wild | LNCaP |
| NO 266 | GGT(Gly) | TAACCCTAA-iAmMC6T-TTAGG | | |
| | | GTTAGGG | | |
| | | GGAGCTGGTGGCGTA | | |
| SEQ ID | K-ras 12 | TAGCTGTATCGTCAAGGCCCC | Mutant | A549 |
| NO 267 | AGT(Ser) | TAACCCTAA-iAmMC6T-TTAGG | | |
| | | GTTAGGG | | |
| | | GGAGCTAGTGGCGTA | | |
| SEQ ID | K-ras 12 | TAGCTGTATCGTCAAGGCCCC | Mutant | H157 |
| NO 268 | CGT(Arg) | TAACCCTAA-iAmMC6T-TTAGG | | |
| | | GTTAGGG | | |
| | | GGAGCTCGTGGCGTA | | |
| SEQ ID | K-ras 12 | TAGCTGTATCGTCAAGGCCCC | Mutant | Mia-PaC |
| NO 269 | TGT(Asp) | TAACCCTAA-iAmMC6T-TTAGG | | a-2 |
| | | GTTAGGG | | |
| | | GGAGCTTGTGGCGTA | | |
| SEQ ID | K-ras 12 | TAGCTGTATCGTCAAGGCCCC | Mutant | LS174T |
| NO 270 | GAT(Asp) | TAACCCTAA-iAmMC6T-TTAGG | | |
| | | GTTAGGG | | |
| | | GGAGCTGATGGCGTA | | |
| SEQ ID | K-ras 12 | TAGCTGTATCGTCAAGGCCCC | Mutant | SW1116 |
| NO 271 | GCT(Ala) | TAACCCTAA-iAmMC6T-TTAGG | | |
| | | GTTAGGGT | | |
| | | GGAGCTGCTGGCGTA | | |
| SEQ ID | K-ras 12 | TAGCTGTATCGTCAAGGCCCC | Mutant | SW620 |
| NO 272 | GTT(Val) | TAACCCTAA-iAmMC6T-TTAGG | | |
| | | GTTAGGG | | |
| | | GGAGCTGTTGGCGTA | | |

Note)
Underlined sequence: probe part

14.2. Preparation of Sample, Isolation of DNA and PCR

After isolating DNA from each sample, PCR was performed in the presence of a fluorescent dye. The sense strand for mutation analysis was labeled with Cy-3 and the antisense strand for the control gene was labeled with Cy-5. After PCR, the mutation site of the target gene is amplified as labeled with Cy-3 and the control gene of the antisense strand is amplified as labeled with Cy-5. The sequence of the forward primer was 5′-Cy-5-GACTGAATATAAACTTGTGG-3′ and the sequence of the reverse primer was 5′-Cy-3-CTATTGTTGGATCATATTCG-3′. PCR was carried out for 35 cycles after initial denaturation at 96° C. for 3 minutes. Each cycle consisted of 30 seconds at 94° C., 30 seconds at 58° C. and 30 seconds at 72° C. Final extension was carried out at 72° C. for 5 minutes.

14.3. Hybridization and Analysis

The PCR product was hybridized and analyzed using a scanner in the same manner as in Example 13.

By normalizing the Cy-3 signal with respect to the Cy-5 signal after removing the background signal from each spot, the spots showing signals above an appropriate cutoff level were found. They are perfectly matching alleles. By doing so, it can be identified whether the target gene is wild type or mutant type as well as its heterozygosity.

When used for spike testing, the K-RAS microarray of the present disclosure can identify mutation if the quantity of the mutant genes is 1% or more of the normal genes.

Those skilled in the art will appreciate that the conceptions and specific embodiments disclosed in the foregoing description may be readily utilized as a basis for modifying or designing other embodiments for carrying out the same purposes of the present disclosure. Those skilled in the art will also appreciate that such equivalent embodiments do not depart from the spirit and scope of the disclosure as set forth in the appended claims.

1. A Y-shaped nucleotide probe having two probe parts in one body.

2. The probe according to claim 1, wherein the probe sequentially comprises, in 5′→3′ direction, from left top to right top, a structure selected from the group consisting of:
a) (1) a left-side probe part, (2) a left-side stem part, (3) a linker part, (4) a right-side stem part and (5) a right-side probe part;
b) only (2) the left-side stem part, (3) the linker part, (4) the right-side stem part and (5) the right-side probe part; or
c) only (1) the left-side probe part, (2) the left-side stem part, (3) the linker part and (4) the right-side stem part.

3. (canceled)

4. (canceled)

5. The probe according to claim 2, wherein the left-side stem part and the right-side stem part are oligonucleotides having complementary nucleotide sequences and bonded together, and each of the left-side stem part and the right-side stem part comprises the nucleotide G in at least half of the entire nucleotide sequence.

6. The probe according to claim 2, wherein the left-side stem part and the right-side stem part are oligonucleotides having complementary nucleotide sequences and bonded together, and the nucleotide sequence of the stem part is a telomere nucleotide sequence.

7. The probe according to claim 5, wherein the left-side stem part or the right-side stem part comprises at least one repeating nucleotide unit selected from a group consisting of TTGGG, TAGGG, TTGGGG, TTTGGG, TTAGGG, TTTGGGG, TTTAGGG, TTTTGGGG and TTTAGGGG.

8. The probe according to claim 2, wherein the left-side probe part or the right-side probe part is an oligonucleotide having a nucleotide sequence complementary to that of a target gene.

9. The probe according to claim 2, wherein the left-side probe part or the right-side probe part is an oligonucleotide comprising 15-150 nucleotide sequences.

10. The probe according to claim 2, wherein the nucleotide sequence of the left-side probe part is in 5′→3′ direction from top to bottom, and the nucleotide sequence of the right-side probe is in 5′→3′ direction from bottom to top.

11. The probe according to claim 2, wherein the linker comprises an amino-modified dideoxythymidine selected from C6dT, C3dT, C12dT and C18dT for binding to an aldehyde-coated solid support.

12. The probe according to claim 2, wherein the probe comprises a peptide nucleic acid (PNA).

13. The probe according to claim 2, which is prepared by a synthesis method comprising 1) a detritylation step, 2) a coupling step, 3) a capping step and 4) an oxidation step.

14. The probe according to claim 2, wherein the left-side probe part and the right-side probe part respectively comprise oligonucleotides having nucleotide sequences complementary to two different regions of a target gene.

15. The probe according to claim 2, wherein the left-side probe part and the right-side probe part respectively comprise oligonucleotides having nucleotide sequences complementary to the same region of a target gene.

16. The probe according to claim 2, wherein the left-side probe part and the right-side probe part respectively comprise oligonucleotides having nucleotide sequences complementary to those of different target genes.

17. The probe according to claim 2, wherein one of the left-side probe part and the right-side probe part comprises an oligonucleotide having a nucleotide sequence complementary to that of a target gene and the other probe part comprises an oligonucleotide having a nucleotide sequence complementary to that of a control gene.

18. The probe according to claim 17, wherein the control gene lacks complementarity to the target gene and is nonexistent or unexpressed in a sample.

19. The probe according to claim 17, wherein the control gene is the motD gene of E. coli.

20. The probe according to claim 2, wherein the probe is an oligonucleotide having at least one nucleotide sequence selected from SEQ ID NOS 5-50.

21. A DNA microarray comprising the probe according to claim 2 directly spotted on a solid support.

22. The DNA microarray according to claim 21, wherein the solid support is selected from a group consisting of a glass slide, a bead, a microplate well, a silicon wafer and a nylon membrane.

23. The DNA microarray according to claim 21, wherein the human beta-globin gene is further spotted on the DNA microarray.

24. The DNA microarray according to claim 21, wherein the DNA microarray has 8 wells for spotting the probe.

25. The DNA microarray according to claim 21, wherein the probe comprises an oligonucleotide having at least one nucleotide sequence selected from SEQ ID NOS 5-50 and is for detecting and genotyping human papilloma virus (HPV).

26. The DNA microarray according to claim 25, wherein the probe binds complementarily to an oligonucleotide primer having a nucleotide sequence of SEQ ID NO 4 with the 5′-terminal labeled with Cy5 and an oligonucleotide primer having a nucleotide sequence of SEQ ID NO 1 with the 5′-terminal labeled with Cy3.

27. The DNA microarray according to claim 21, wherein the probe comprises an oligonucleotide having a nucleotide sequence selected from SEQ ID NOS 51-55 and is for detecting and genotyping Neisseria gonorrhoeae (NG), Chlamydia trachomatis (CT), herpes simplex virus (HSV), Treponema pallidum (TP) and Haemophilus ducreyi (HD) as a pathogen causing a sexually transmitted disease (STD), respectively.

28. The DNA microarray according to claim 21, wherein the probe comprises an oligonucleotide having a nucleotide sequence selected from SEQ ID NOS 56-199 and is for detecting and genotyping influenza A virus.

29. The DNA microarray according to claim 21, wherein the probe comprises an oligonucleotide having a nucleotide sequence selected from SEQ ID NOS 212-213 and is for analyzing expression of the β-actin or epidermal growth factor receptor (EGFR) gene.

30. The DNA microarray according to claim 21, wherein one of the left-side probe part and the right-side probe part of the probe comprises an oligonucleotide complementary to the single nucleotide polymorphism (SNP) site of a sense strand of a target nucleic acid and the other comprises an oligonucleotide complementary to an antisense strand of the target nucleic acid lacking the SNP site, and the probe is for analyzing SNP.

31. The DNA microarray according to claim 30, wherein the probe comprises an oligonucleotide having at least one nucleotide sequence selected from SEQ ID NOS 220-239 and is for analyzing SNP of the ACE, ADRB2, Apo E, CETP, CFH, ESR1, IL1A, MTHFR or NOS3 gene.

32. The DNA microarray according to claim 21, wherein the probe comprises an oligonucleotide having at least one nucleotide sequence selected from SEQ ID NOS 258-272 and is for analyzing mutation of the K-ras gene.

33. The DNA microarray according to claim 21, wherein the right-side probe part of the d-shaped probe comprises an oligonucleotide having a nucleotide sequence complementary to point mutation of A, C, G or T, the nucleotide complementary to the point mutation is located at the center of the right-side probe part, the right-side probe part is 15-30 bp in length, and the d-shaped probe is for analyzing point mutation.

34. A kit for genetic analysis of a sample comprising the DNA microarray according to claim 21, a primer set and a buffer for PCR of a target gene in the sample, and a hybridization buffer.

35. The kit according to claim 34, wherein the primer set for PCR is an oligonucleotide having a nucleotide sequence selected from SEQ ID NOS 208-211 for amplification of the gene of influenza A virus.

36. The kit according to claim 34, wherein the primer set for PCR is an oligonucleotide having a nucleotide sequence of SEQ ID NOS 214-215 or 217-218 for quantitative real-time PCR of the β-actin and EGFR genes, respectively.

37. The kit according to claim 34, wherein the primer set for PCR is an oligonucleotide having at least two nucleotide sequences selected from SEQ ID NOS 240-257 for detection of SNP.

38. The kit according to claim 34, wherein the kit is for diagnosis, prevention, prognosis or personalized therapy of a disease.

39. A genetic analysis method comprising placing a target nucleic acid of a sample labeled with a label on the DNA microarray according to claim 21 and hybridizing the probe of the DNA microarray with the target nucleic acid.

40. The genetic analysis method according to claim 39, wherein the label is at least one selected from a group consisting of Cy3, Cy5, Cy5.5, BODIPY, Alexa 488, Alexa 532, Alexa 546, Alexa 568, Alexa 594, Alexa 660, rhodamine, TAMRA, FAM, FITC, Fluor X, ROX, Texas Red, Orange Green 488X, Orange green 514X, HEX, TET, JOE, Oyster 556, Oyster 645, BODIPY 630/650, BODIPY 650/665, Cal Fluor Orange 546, Cal Fluor Red 610, Quasar 670 and biotin.

41. The genetic analysis method according to claim 39, wherein the target nucleic acid is labeled with the label by PCR, RT-PCR or in vitro transcription.

42. The genetic analysis method according to claim 39, wherein the genetic analysis method further comprises, after the hybridization, investigating the expression level of the target nucleic acid by analyzing a signal from the label using a fluorescence scanner.

43. The genetic analysis method according to claim 42, wherein the signal is analyzed through normalization.

44. The genetic analysis method according to claim 43, wherein the normalization comprises a triple normalization procedure of investigating Cy5 and Cy3 signals from each spot excluding the background noise signal and comparing with the Cy3 signal from the β-actin gene as a housekeeping gene.

45. The genetic analysis method according to claim 39, wherein the target nucleic acid is selected from a group consisting of DNA, RNA, cDNA and cRNA.

46. The genetic analysis method according to claim 45, wherein the cDNA is labeled with Cy3 by RT-PCT and the cRNA is labeled with Cy3 by in vitro transcription.

47. The genetic analysis method according to claim 46, wherein the cDNA or cRNA labeled with Cy3 is mixed with the motD gene of E. coli labeled with Cy5 as an external control and the resulting mixture is hybridized.


SEQUENCE LISTING (272 sequences)

SEQ ID NO: 1 - DNA, 20 bp, Artificial Sequence
  Primer HBB H1
acacaactgtgttcactagc

SEQ ID NO: 2 - DNA, 21 bp, Artificial Sequence
  Primer for HBB H2
caaacttcatccacgttcacc

SEQ ID NO: 3 - DNA, 20 bp, Artificial Sequence
  Forward Primer for HPV L1
gcmcagggwcataayaatgg

SEQ ID NO: 4 - DNA, 24 bp, Artificial Sequence
  Reverse Primer for HPV L1
aataaactgtaaatcatattcctc

SEQ ID NO: 5 - DNA, 51 bp, Artificial Sequence
  Probe for HPV6
cggcagacttctcctcccctaanttaggggcatccgtaactacatcttcca

SEQ ID NO: 6 - DNA, 51 bp, Artificial Sequence
  Probe for HPV7
cggcagacttctcctcccctaanttagggacaccaacaccatatgacaata

SEQ ID NO: 7 - DNA, 47 bp, Artificial Sequence
  Probe for HPV10
cggcagacttctcctcccctaanttaggggcctcccctgccactacg

SEQ ID NO: 8 - DNA, 47 bp, Artificial Sequence
  Probe for HPV11
cggcagacttctcctcccctaanttagggatttgctggggaaaccac

SEQ ID NO: 9 - DNA, 51 bp, Artificial Sequence
  Probe for HPV16
cggcagacttctcctcccctaanttagggtgccatatctacttcagaaact

SEQ ID NO: 10 - DNA, 50 bp, Artificial Sequence
  Probe for HPV18
cggcagacttctcctcccctaanttagggtctacacagtctccgtacctg

SEQ ID NO: 11 - DNA, 51 bp, Artificial Sequence
  Probe for HPV26
cggcagacttctcctcccctaanttagggattatctgcagcatctgcatcc

SEQ ID NO: 12 - DNA, 55 bp, Artificial Sequence
  Probe for HPV27
cggcagacttctcctcccctaanttagggcagctgaggtgtctgataatactaat

SEQ ID NO: 13 - DNA, 49 bp, Artificial Sequence
  Probe for HPV30
cggcagacttctcctcccctaanttagggaaccacacaaacgttatcca

SEQ ID NO: 14 - DNA, 51 bp, Artificial Sequence
  Probe for HPV31
cggcagacttctcctcccctaanttagggctgcaattgcaaacagtgatac

SEQ ID NO: 15 - DNA, 54 bp, Artificial Sequence
  Probe for HPV32
cggcagacttctcctcccctaanttaggggacacatacaagtctactaacttta

SEQ ID NO: 16 - DNA, 54 bp, Artificial Sequence
  Probe for HPV33
cggcagacttctcctcccctaanttaggggcacacaagtaactagtgacagtac

SEQ ID NO: 17 - DNA, 48 bp, Artificial Sequence
  Probe for HPV34
cggcagacttctcctcccctaanttagggccacaagtacaactgcacc

SEQ ID NO: 18 - DNA, 54 bp, Artificial Sequence
  Probe for HPV35
cggcagacttctcctcccctaanttagggtctgctgtgtcttctagtgacagta

SEQ ID NO: 19 - DNA, 58 bp, Artificial Sequence
  Probe for HPV39
cggcagacttctcctcccctaanttagggacctctatagagtcttccataccttctac

SEQ ID NO: 20 - DNA, 45 bp, Artificial Sequence
  Probe for HPV40
cggcagacttctcctcccctaanttagggagtcccccacaccaac

SEQ ID NO: 21 - DNA, 47 bp, Artificial Sequence
  Probe for HPV42
cggcagacttctcctcccctaanttagggcactgcaacatctggtga

SEQ ID NO: 22 - DNA, 51 bp, Artificial Sequence
  Probe for HPV43
cggcagacttctcctcccctaanttaggggcccagtacatatgacaatgca

SEQ ID NO: 23 - DNA, 47 bp, Artificial Sequence
  Probe for HPV44
cggcagacttctcctcccctaanttagggtacacagtcccctccgtc

SEQ ID NO: 24 - DNA, 48 bp, Artificial Sequence
  Probe for HPV45
cggcagacttctcctcccctaanttagggcacaaaatcctgtgccaag

SEQ ID NO: 25 - DNA, 49 bp, Artificial Sequence
  Probe for HPV51
cggcagacttctcctcccctaanttagggggtttccccaacatttactc

SEQ ID NO: 26 - DNA, 51 bp, Artificial Sequence
  Probe for HPV52
cggcagacttctcctcccctaanttaggggctgaggttaaaaaggaaagca

SEQ ID NO: 27 - DNA, 52 bp, Artificial Sequence
  Probe for HPV53
cggcagacttctcctcccctaanttagggcgcaaccacacagtctatgtcta

SEQ ID NO: 28 - DNA, 46 bp, Artificial Sequence
  Probe for HPV54
cggcagacttctcctcccctaanttagggtacagcatccacgcagg

SEQ ID NO: 29 - DNA, 53 bp, Artificial Sequence
  Probe for HPV55
cggcagacttctcctcccctaanttagggctacaactcagtctccatctacaa

SEQ ID NO: 30 - DNA, 63 bp, Artificial Sequence
  Probe for HPV56
cggcagacttctcctcccctaanttaggggactattagtactgctacagaacagttaagtaaa

SEQ ID NO: 31 - DNA, 53 bp, Artificial Sequence
  Probe for HPV57
cggcagacttctcctcccctaanttagggccactgtaaccacagaaactaatt

SEQ ID NO: 32 - DNA, 52 bp, Artificial Sequence
  Probe for HPV58
cggcagacttctcctcccctaanttagggtgcactgaagtaactaaggaagg

SEQ ID NO: 33 - DNA, 58 bp, Artificial Sequence
  Probe for HPV59
cggcagacttctcctcccctaanttagggtctattcctaatgtatacacacctaccag

SEQ ID NO: 34 - DNA, 49 bp, Artificial Sequence
  Probe for HPV61
cggcagacttctcctcccctaanttagggtgctacatccccccctgtat

SEQ ID NO: 35 - DNA, 49 bp, Artificial Sequence
  Probe for HPV62
cggcagacttctcctcccctaanttagggactatttgtaccgcctccac

SEQ ID NO: 36 - DNA, 55 bp, Artificial Sequence
  Probe for HPV66
cggcagacttctcctcccctaanttagggaatgcagctaaaagcacattaactaa

SEQ ID NO: 37 - DNA, 52 bp, Artificial Sequence
  Probe for HPV67
cggcagacttctcctcccctaanttagggaaaatcagaggctacatacaaaa

SEQ ID NO: 38 - DNA, 60 bp, Artificial Sequence
  Probe for HPV68b
cggcagacttctcctcccctaanttagggctactactactgaatcagctgtaccaaatat

SEQ ID NO: 39 - DNA, 52 bp, Artificial Sequence
  Probe for HPV68a
cggcagacttctcctcccctaanttagggcagactctactgtaccagctgtg

SEQ ID NO: 40 - DNA, 54 bp, Artificial Sequence
  Probe for HPV69
cggcagacttctcctcccctaanttagggcacaatctgcatctgccacttttaa

SEQ ID NO: 41 - DNA, 46 bp, Artificial Sequence
  Probe for HPV70
cggcagacttctcctcccctaanttagggccgaaacggccatacct

SEQ ID NO: 42 - DNA, 50 bp, Artificial Sequence
  Probe for HPV72
cggcagacttctcctcccctaanttagggcacagcgtcctctgtatcaga

SEQ ID NO: 43 - DNA, 56 bp, Artificial Sequence
  Probe for HPV73
cggcagacttctcctcccctaanttagggaggtacacaggctagtagctctactac

SEQ ID NO: 44 - DNA, 49 bp, Artificial Sequence
  Probe for HPV81
cggcagacttctcctcccctaanttaggggctacatctgctgctgcaga

SEQ ID NO: 45 - DNA, 48 bp, Artificial Sequence
  Probe for HPV82
cggcagacttctcctcccctaanttagggctccagcaaactttaagca

SEQ ID NO: 46 - DNA, 50 bp, Artificial Sequence
  Probe for HPV83
cggcagacttctcctcccctaanttagggtgctgctacacaggctaatga

SEQ ID NO: 47 - DNA, 56 bp, Artificial Sequence
  Probe for HPV84
cggcagacttctcctcccctaanttagggaccgaatcagaatataaacctaccaat

SEQ ID NO: 48 - DNA, 52 bp, Artificial Sequence
  Probe for HPV90
cggcagacttctcctcccctaanttagggacaaacaccctctgacacataca

SEQ ID NO: 49 - DNA, 57 bp, Artificial Sequence
  Probe for HPV91
cggcagacttctcctcccctaanttagggtctgtgctacctactacatatgacaaca

SEQ ID NO: 50 - DNA, 59 bp, Artificial Sequence
  Probe for HPVU
cggcagacttctcctcccctaanttagggttgttgggdtaatcagttgtttgttactgt

SEQ ID NO: 51 - DNA, 68 bp, Artificial Sequence
  Probe for Neisseria Gonorrhea
caacaaacgaaagcagacttagagaccccctaanttaggggatatttttccgtaacgtctctaagtct

SEQ ID NO: 52 - DNA, 64 bp, Artificial Sequence
  Probe for Chlamydia Trachomatis
gttcgttgtagagccatgtcctatccccctaanttagggttttcttcgtcagttaaaccttccc

SEQ ID NO: 53 - DNA, 48 bp, Artificial Sequence
  Probe for Herpes Simplex Virus
gcccccggggtcggaagcccctaanttagggaccccaccagcccggac

SEQ ID NO: 54 - DNA, 62 bp, Artificial Sequence
  Probe for Treponema Pallidum
acgtaaggtaagcagcatggagacccctaanttagggacgtgcagaaaaactatcctcagtg

SEQ ID NO: 55 - DNA, 66 bp, Artificial Sequence
  Probe for Hemophilus Ducreyi
gaagatattacgcggtattagctacacccctaanttaggggtgagtaatgcttgggaatctggctt

SEQ ID NO: 56 - DNA, 62 bp, Artificial Sequence
  Probe for Influenza A Virus H1N1
cagaaattccaattgtcaaccaactccctaanttagggtgcttatgtctctgtagtgtcttc

SEQ ID NO: 57 - DNA, 61 bp, Artificial Sequence
  Probe for Influenza A Virus H1N2
tacaagttccattgatacaaacgcccctaanttagggtgcttatgtctctgtagtgtcttc

SEQ ID NO: 58 - DNA, 59 bp, Artificial Sequence
  Probe for Influenza A Virus H1N3
cccttccaattgtccctacataccctaanttagggtgcttatgtctctgtagtgtcttc

SEQ ID NO: 59 - DNA, 62 bp, Artificial Sequence
  Probe for Influenza A Virus H1N4
caaatatcccactacatacatatccccctaanttagggtgcttatgtctctgtagtgtcttc

SEQ ID NO: 60 - DNA, 61 bp, Artificial Sequence
  Probe for Influenza A Virus H1N5
ccattccaattatctcggcaaaccccctaanttagggtgcttatgtctctgtagtgtcttc

SEQ ID NO: 61 - DNA, 61 bp, Artificial Sequence
  Probe for Influenza A Virus H1N6
tgagtccttaatatatttcctgccccctaanttagggtgcttatgtctctgtagtgtcttc

SEQ ID NO: 62 - DNA, 60 bp, Artificial Sequence
  Probe for Influenza A Virus H1N7
cctgcattaggtattttcaacatccctaanttagggtgcttatgtctctgtagtgtcttc

SEQ ID NO: 63 - DNA, 57 bp, Artificial Sequence
  Probe for Influenza A Virus H1N8
ccacacatcacaatggagctccctaanttagggtgcttatgtctctgtagtgtcttc

SEQ ID NO: 64 - DNA, 60 bp, Artificial Sequence
  Probe for Influenza A Virus H1N9
ttgtatagtgtgggtggtgatgaccctaanttagggtgcttatgtctctgtagtgtcttc

SEQ ID NO: 65 - DNA, 61 bp, Artificial Sequence
  Probe for Influenza A Virus H2N1
cagaaattccaattgtcaaccaactccctaanttagggacatcaacactgaataagaggtc

SEQ ID NO: 66 - DNA, 60 bp, Artificial Sequence
  Probe for Influenza A Virus H2N2
tacaagttccattgatacaaacgcccctaanttagggacatcaacactgaataagaggtc

SEQ ID NO: 67 - DNA, 58 bp, Artificial Sequence
  Probe for Influenza A Virus H2N3
cccttccaattgtccctacataccctaanttagggacatcaacactgaataagaggtc

SEQ ID NO: 68 - DNA, 61 bp, Artificial Sequence
  Probe for Influenza A Virus H2N4
caaatatcccactacatacatatccccctaanttagggacatcaacactgaataagaggtc

SEQ ID NO: 69 - DNA, 60 bp, Artificial Sequence
  Probe for Influenza A Virus H2N5
ccattccaattatctcggcaaaccccctaanttagggacatcaacactgaataagaggtc

SEQ ID NO: 70 - DNA, 60 bp, Artificial Sequence
  Probe for Influenza A Virus H2N6
tgagtccttaatatatttcctgccccctaanttagggacatcaacactgaataagaggtc

SEQ ID NO: 71 - DNA, 59 bp, Artificial Sequence
  Probe for Influenza A Virus H2N7
cctgcattaggtattttcaacatccctaanttagggacatcaacactgaataagaggtc

SEQ ID NO: 72 - DNA, 56 bp, Artificial Sequence
  Probe for Influenza A Virus H2N8
ccacacatcacaatggagctccctaanttagggacatcaacactgaataagaggtc

SEQ ID NO: 73 - DNA, 59 bp, Artificial Sequence
  Probe for Influenza A Virus H2N9
ttgtatagtgtgggtggtgatgaccctaanttagggacatcaacactgaataagaggtc

SEQ ID NO: 74 - DNA, 61 bp, Artificial Sequence
  Probe for Influenza A Virus H3N1
cagaaattccaattgtcaaccaactccctaanttagggcctcggggttacttcaaaatacg

SEQ ID NO: 75 - DNA, 60 bp, Artificial Sequence
  Probe for Influenza A Virus H3N2
tacaagttccattgatacaaacgcccctaanttagggcctcggggttacttcaaaatacg

SEQ ID NO: 76 - DNA, 58 bp, Artificial Sequence
  Probe for Influenza A Virus H3N3
cccttccaattgtccctacataccctaanttagggcctcggggttacttcaaaatacg

SEQ ID NO: 77 - DNA, 61 bp, Artificial Sequence
  Probe for Influenza A Virus H3N4
caaatatcccactacatacatatccccctaanttagggcctcggggttacttcaaaatacg

SEQ ID NO: 78 - DNA, 60 bp, Artificial Sequence
  Probe for Influenza A Virus H3N5
ccattccaattatctcggcaaaccccctaanttagggcctcggggttacttcaaaatacg

SEQ ID NO: 79 - DNA, 60 bp, Artificial Sequence
  Probe for Influenza A Virus H3N6
tgagtccttaatatatttcctgccccctaanttagggcctcggggttacttcaaaatacg

SEQ ID NO: 80 - DNA, 59 bp, Artificial Sequence
  Probe for Influenza A Virus H3N7
cctgcattaggtattttcaacatccctaanttagggcctcggggttacttcaaaatacg

SEQ ID NO: 81 - DNA, 56 bp, Artificial Sequence
  Probe for Influenza A Virus H3N8
ccacacatcacaatggagctccctaanttagggcctcggggttacttcaaaatacg

SEQ ID NO: 82 - DNA, 59 bp, Artificial Sequence
  Probe for Influenza A Virus H3N9
ttgtatagtgtgggtggtgatgaccctaanttagggcctcggggttacttcaaaatacg

SEQ ID NO: 83 - DNA, 59 bp, Artificial Sequence
  Probe for Influenza A Virus H4N1
cagaaattccaattgtcaaccaactccctaanttaggggacaaaggtcaacaatgggga

SEQ ID NO: 84 - DNA, 58 bp, Artificial Sequence
  Probe for Influenza A Virus H4N2
tacaagttccattgatacaaacgcccctaanttaggggacaaaggtcaacaatgggga

SEQ ID NO: 85 - DNA, 56 bp, Artificial Sequence
  Probe for Influenza A Virus H4N3
cccttccaattgtccctacataccctaanttaggggacaaaggtcaacaatgggga

SEQ ID NO: 86 - DNA, 59 bp, Artificial Sequence
  Probe for Influenza A Virus H4N4
caaatatcccactacatacatatccccctaanttaggggacaaaggtcaacaatgggga

SEQ ID NO: 87 - DNA, 58 bp, Artificial Sequence
  Probe for Influenza A Virus H4N5
ccattccaattatctcggcaaaccccctaanttaggggacaaaggtcaacaatgggga

SEQ ID NO: 88 - DNA, 58 bp, Artificial Sequence
  Probe for Influenza A Virus H4N6
tgagtccttaatatatttcctgccccctaanttaggggacaaaggtcaacaatgggga

SEQ ID NO: 89 - DNA, 57 bp, Artificial Sequence
  Probe for Influenza A Virus H4N7
cctgcattaggtattttcaacatccctaanttaggggacaaaggtcaacaatgggga

SEQ ID NO: 90 - DNA, 54 bp, Artificial Sequence
  Probe for Influenza A Virus H4N8
ccacacatcacaatggagctccctaanttaggggacaaaggtcaacaatgggga

SEQ ID NO: 91 - DNA, 57 bp, Artificial Sequence
  Probe for Influenza A Virus H4N9
ttgtatagtgtgggtggtgatgaccctaanttaggggacaaaggtcaacaatgggga

SEQ ID NO: 92 - DNA, 62 bp, Artificial Sequence
  Probe for Influenza A Virus H5N1
cagaaattccaattgtcaaccaactccctaanttaggggtcaccaataaggtcaactcgatc

SEQ ID NO: 93 - DNA, 61 bp, Artificial Sequence
  Probe for Influenza A Virus H5N2
tacaagttccattgatacaaacgcccctaanttaggggtcaccaataaggtcaactcgatc

SEQ ID NO: 94 - DNA, 59 bp, Artificial Sequence
  Probe for Influenza A Virus H5N3
cccttccaattgtccctacataccctaanttaggggtcaccaataaggtcaactcgatc

SEQ ID NO: 95 - DNA, 62 bp, Artificial Sequence
  Probe for Influenza A Virus H5N4
caaatatcccactacatacatatccccctaanttaggggtcaccaataaggtcaactcgatc

SEQ ID NO: 96 - DNA, 61 bp, Artificial Sequence
  Probe for Influenza A Virus H5N5
ccattccaattatctcggcaaaccccctaanttaggggtcaccaataaggtcaactcgatc

SEQ ID NO: 97 - DNA, 61 bp, Artificial Sequence
  Probe for Influenza A Virus H5N6
tgagtccttaatatatttcctgccccctaanttaggggtcaccaataaggtcaactcgatc

SEQ ID NO: 98 - DNA, 60 bp, Artificial Sequence
  Probe for Influenza A Virus H5N7
cctgcattaggtattttcaacatccctaanttaggggtcaccaataaggtcaactcgatc

SEQ ID NO: 99 - DNA, 57 bp, Artificial Sequence
  Probe for Influenza A Virus H5N8
ccacacatcacaatggagctccctaanttaggggtcaccaataaggtcaactcgatc

SEQ ID NO: 100 - DNA, 60 bp, Artificial Sequence
  Probe for Influenza A Virus H5N9
ttgtatagtgtgggtggtgatgaccctaanttaggggtcaccaataaggtcaactcgatc

SEQ ID NO: 101 - DNA, 63 bp, Artificial Sequence
  Probe for Influenza A Virus H6N1
cagaaattccaattgtcaaccaactccctaanttagggtgagatgtttcccaaaagtacatgg

SEQ ID NO: 102 - DNA, 62 bp, Artificial Sequence
  Probe for Influenza A Virus H6N2
tacaagttccattgatacaaacgcccctaanttagggtgagatgtttcccaaaagtacatgg

SEQ ID NO: 103 - DNA, 60 bp, Artificial Sequence
  Probe for Influenza A Virus H6N3
cccttccaattgtccctacataccctaanttagggtgagatgtttcccaaaagtacatgg

SEQ ID NO: 104 - DNA, 63 bp, Artificial Sequence
  Probe for Influenza A Virus H6N4
caaatatcccactacatacatatccccctaanttagggtgagatgtttcccaaaagtacatgg

SEQ ID NO: 105 - DNA, 62 bp, Artificial Sequence
  Probe for Influenza A Virus H6N5
ccattccaattatctcggcaaaccccctaanttagggtgagatgtttcccaaaagtacatgg

SEQ ID NO: 106 - DNA, 62 bp, Artificial Sequence
  Probe for Influenza A Virus H6N6
tgagtccttaatatatttcctgccccctaanttagggtgagatgtttcccaaaagtacatgg

SEQ ID NO: 107 - DNA, 61 bp, Artificial Sequence
  Probe for Influenza A Virus H6N7
cctgcattaggtattttcaacatccctaanttagggtgagatgtttcccaaaagtacatgg

SEQ ID NO: 108 - DNA, 58 bp, Artificial Sequence
  Probe for Influenza A Virus H6N8
ccacacatcacaatggagctccctaanttagggtgagatgtttcccaaaagtacatgg

SEQ ID NO: 109 - DNA, 61 bp, Artificial Sequence
  Probe for Influenza A Virus H6N9
ttgtatagtgtgggtggtgatgaccctaanttagggtgagatgtttcccaaaagtacatgg

SEQ ID NO: 110 - DNA, 62 bp, Artificial Sequence
  Probe for Influenza A Virus H7N1
cagaaattccaattgtcaaccaactccctaanttagggcagaccaaactctatggaagtgga

SEQ ID NO: 111 - DNA, 61 bp, Artificial Sequence
  Probe for Influenza A Virus H7N2
tacaagttccattgatacaaacgcccctaanttagggcagaccaaactctatggaagtgga

SEQ ID NO: 112 - DNA, 59 bp, Artificial Sequence
  Probe for Influenza A Virus H7N3
cccttccaattgtccctacataccctaanttagggcagaccaaactctatggaagtgga

SEQ ID NO: 113 - DNA, 62 bp, Artificial Sequence
  Probe for Influenza A Virus H7N4
caaatatcccactacatacatatccccctaanttagggcagaccaaactctatggaagtgga

SEQ ID NO: 114 - DNA, 61 bp, Artificial Sequence
  Probe for Influenza A Virus H7N5
ccattccaattatctcggcaaaccccctaanttagggcagaccaaactctatggaagtgga

SEQ ID NO: 115 - DNA, 61 bp, Artificial Sequence
  Probe for Influenza A Virus H7N6
tgagtccttaatatatttcctgccccctaanttagggcagaccaaactctatggaagtgga

SEQ ID NO: 116 - DNA, 60 bp, Artificial Sequence
  Probe for Influenza A Virus H7N7
cctgcattaggtattttcaacatccctaanttagggcagaccaaactctatggaagtgga

SEQ ID NO: 117 - DNA, 57 bp, Artificial Sequence
  Probe for Influenza A Virus H7N8
ccacacatcacaatggagctccctaanttagggcagaccaaactctatggaagtgga

SEQ ID NO: 118 - DNA, 60 bp, Artificial Sequence
  Probe for Influenza A Virus H7N9
ttgtatagtgtgggtggtgatgaccctaanttagggcagaccaaactctatggaagtgga

SEQ ID NO: 119 - DNA, 61 bp, Artificial Sequence
  Probe for Influenza A Virus H8N1
cagaaattccaattgtcaaccaactccctaanttagggtggagacatcattttcttatggg

SEQ ID NO: 120 - DNA, 60 bp, Artificial Sequence
  Probe for Influenza A Virus H8N2
tacaagttccattgatacaaacgcccctaanttagggtggagacatcattttcttatggg

SEQ ID NO: 121 - DNA, 58 bp, Artificial Sequence
  Probe for Influenza A Virus H8N3
cccttccaattgtccctacataccctaanttagggtggagacatcattttcttatggg

SEQ ID NO: 122 - DNA, 61 bp, Artificial Sequence
  Probe for Influenza A Virus H8N4
caaatatcccactacatacatatccccctaanttagggtggagacatcattttcttatggg

SEQ ID NO: 123 - DNA, 60 bp, Artificial Sequence
  Probe for Influenza A Virus H8N5
ccattccaattatctcggcaaaccccctaanttagggtggagacatcattttcttatggg

SEQ ID NO: 124 - DNA, 60 bp, Artificial Sequence
  Probe for Influenza A Virus H8N6
tgagtccttaatatatttcctgccccctaanttagggtggagacatcattttcttatggg

SEQ ID NO: 125 - DNA, 59 bp, Artificial Sequence
  Probe for Influenza A Virus H8N7
cctgcattaggtattttcaacatccctaanttagggtggagacatcattttcttatggg

SEQ ID NO: 126 - DNA, 56 bp, Artificial Sequence
  Probe for Influenza A Virus H8N8
ccacacatcacaatggagctccctaanttagggtggagacatcattttcttatggg

SEQ ID NO: 127 - DNA, 59 bp, Artificial Sequence
  Probe for Influenza A Virus H8N9
ttgtatagtgtgggtggtgatgaccctaanttagggtggagacatcattttcttatggg

SEQ ID NO: 128 - DNA, 62 bp, Artificial Sequence
  Probe for Influenza A Virus H9N1
cagaaattccaattgtcaaccaactccctaanttagggcaagacgcccaatacacaaataat

SEQ ID NO: 129 - DNA, 61 bp, Artificial Sequence
  Probe for Influenza A Virus H9N2
tacaagttccattgatacaaacgcccctaanttagggcaagacgcccaatacacaaataat

SEQ ID NO: 130 - DNA, 59 bp, Artificial Sequence
  Probe for Influenza A Virus H9N3
cccttccaattgtccctacataccctaanttagggcaagacgcccaatacacaaataat

SEQ ID NO: 131 - DNA, 62 bp, Artificial Sequence
  Probe for Influenza A Virus H9N4
caaatatcccactacatacatatccccctaanttagggcaagacgcccaatacacaaataat

SEQ ID NO: 132 - DNA, 61 bp, Artificial Sequence
  Probe for Influenza A Virus H9N5
ccattccaattatctcggcaaaccccctaanttagggcaagacgcccaatacacaaataat

SEQ ID NO: 133 - DNA, 61 bp, Artificial Sequence
  Probe for Influenza A Virus H9N6
tgagtccttaatatatttcctgccccctaanttagggcaagacgcccaatacacaaataat

SEQ ID NO: 134 - DNA, 60 bp, Artificial Sequence
  Probe for Influenza A Virus H9N7
cctgcattaggtattttcaacatccctaanttagggcaagacgcccaatacacaaataat

SEQ ID NO: 135 - DNA, 57 bp, Artificial Sequence
  Probe for Influenza A Virus H9N8
ccacacatcacaatggagctccctaanttagggcaagacgcccaatacacaaataat

SEQ ID NO: 136 - DNA, 60 bp, Artificial Sequence
  Probe for Influenza A Virus H9N9
ttgtatagtgtgggtggtgatgaccctaanttagggcaagacgcccaatacacaaataat

SEQ ID NO: 137 - DNA, 60 bp, Artificial Sequence
  Probe for Influenza A Virus H10N1
cagaaattccaattgtcaaccaactccctaanttagggaaaacaactttgtgcctgtggt

SEQ ID NO: 138 - DNA, 59 bp, Artificial Sequence
  Probe for Influenza A Virus H10N2
tacaagttccattgatacaaacgcccctaanttagggaaaacaactttgtgcctgtggt

SEQ ID NO: 139 - DNA, 57 bp, Artificial Sequence
  Probe for Influenza A Virus H10N3
cccttccaattgtccctacataccctaanttagggaaaacaactttgtgcctgtggt

SEQ ID NO: 140 - DNA, 60 bp, Artificial Sequence
  Probe for Influenza A Virus H10N4
caaatatcccactacatacatatccccctaanttagggaaaacaactttgtgcctgtggt

SEQ ID NO: 141 - DNA, 59 bp, Artificial Sequence
  Probe for Influenza A Virus H10N5
ccattccaattatctcggcaaaccccctaanttagggaaaacaactttgtgcctgtggt

SEQ ID NO: 142 - DNA, 59 bp, Artificial Sequence
  Probe for Influenza A Virus H10N6
tgagtccttaatatatttcctgccccctaanttagggaaaacaactttgtgcctgtggt

SEQ ID NO: 143 - DNA, 58 bp, Artificial Sequence
  Probe for Influenza A Virus H10N7
cctgcattaggtattttcaacatccctaanttagggaaaacaactttgtgcctgtggt

SEQ ID NO: 144 - DNA, 55 bp, Artificial Sequence
  Probe for Influenza A Virus H10N8
ccacacatcacaatggagctccctaanttagggaaaacaactttgtgcctgtggt

SEQ ID NO: 145 - DNA, 58 bp, Artificial Sequence
  Probe for Influenza A Virus H10N9
ttgtatagtgtgggtggtgatgaccctaanttagggaaaacaactttgtgcctgtggt

SEQ ID NO: 146 - DNA, 64 bp, Artificial Sequence
  Probe for Influenza A Virus H11N1
cagaaattccaattgtcaaccaactccctaanttagggcagtgaaatagaggagaggataaacc

SEQ ID NO: 147 - DNA, 63 bp, Artificial Sequence
  Probe for Influenza A Virus H11N2
tacaagttccattgatacaaacgcccctaanttagggcagtgaaatagaggagaggataaacc

SEQ ID NO: 148 - DNA, 61 bp, Artificial Sequence
  Probe for Influenza A Virus H11N3
cccttccaattgtccctacataccctaanttagggcagtgaaatagaggagaggataaacc

SEQ ID NO: 149 - DNA, 64 bp, Artificial Sequence
  Probe for Influenza A Virus H11N4
caaatatcccactacatacatatccccctaanttagggcagtgaaatagaggagaggataaacc

SEQ ID NO: 150 - DNA, 63 bp, Artificial Sequence
  Probe for Influenza A Virus H11N5
ccattccaattatctcggcaaaccccctaanttagggcagtgaaatagaggagaggataaacc

SEQ ID NO: 151 - DNA, 63 bp, Artificial Sequence
  Probe for Influenza A Virus H11N6
tgagtccttaatatatttcctgccccctaanttagggcagtgaaatagaggagaggataaacc

SEQ ID NO: 152 - DNA, 62 bp, Artificial Sequence
  Probe for Influenza A Virus H11N7
cctgcattaggtattttcaacatccctaanttagggcagtgaaatagaggagaggataaacc

SEQ ID NO: 153 - DNA, 59 bp, Artificial Sequence
  Probe for Influenza A Virus H11N8
ccacacatcacaatggagctccctaanttagggcagtgaaatagaggagaggataaacc

SEQ ID NO: 154 - DNA, 62 bp, Artificial Sequence
  Probe for Influenza A Virus H11N9
ttgtatagtgtgggtggtgatgaccctaanttagggcagtgaaatagaggagaggataaacc

SEQ ID NO: 155 - DNA, 61 bp, Artificial Sequence
  Probe for Influenza A Virus H12N1
cagaaattccaattgtcaaccaactccctaanttagggtaatcacagggaaatcacatggc

SEQ ID NO: 156 - DNA, 60 bp, Artificial Sequence
  Probe for Influenza A Virus H12N2
tacaagttccattgatacaaacgcccctaanttagggtaatcacagggaaatcacatggc

SEQ ID NO: 157 - DNA, 58 bp, Artificial Sequence
  Probe for Influenza A Virus H12N3
cccttccaattgtccctacataccctaanttagggtaatcacagggaaatcacatggc

SEQ ID NO: 158 - DNA, 61 bp, Artificial Sequence
  Probe for Influenza A Virus H12N4
caaatatcccactacatacatatccccctaanttagggtaatcacagggaaatcacatggc

SEQ ID NO: 159 - DNA, 60 bp, Artificial Sequence
  Probe for Influenza A Virus H12N5
ccattccaattatctcggcaaaccccctaanttagggtaatcacagggaaatcacatggc

SEQ ID NO: 160 - DNA, 60 bp, Artificial Sequence
  Probe for Influenza A Virus H12N6
tgagtccttaatatatttcctgccccctaanttagggtaatcacagggaaatcacatggc

SEQ ID NO: 161 - DNA, 59 bp, Artificial Sequence
  Probe for Influenza A Virus H12N7
cctgcattaggtattttcaacatccctaanttagggtaatcacagggaaatcacatggc

SEQ ID NO: 162 - DNA, 56 bp, Artificial Sequence
  Probe for Influenza A Virus H12N8
ccacacatcacaatggagctccctaanttagggtaatcacagggaaatcacatggc

SEQ ID NO: 163 - DNA, 59 bp, Artificial Sequence
  Probe for Influenza A Virus H12N9
ttgtatagtgtgggtggtgatgaccctaanttagggtaatcacagggaaatcacatggc

SEQ ID NO: 164 - DNA, 64 bp, Artificial Sequence
  Probe for Influenza A Virus H13N1
cagaaattccaattgtcaaccaactccctaanttagggggatgaagatttactggtatttgatg

SEQ ID NO: 165 - DNA, 63 bp, Artificial Sequence
  Probe for Influenza A Virus H13N2
tacaagttccattgatacaaacgcccctaanttagggggatgaagatttactggtatttgatg

SEQ ID NO: 166 - DNA, 61 bp, Artificial Sequence
  Probe for Influenza A Virus H13N3
cccttccaattgtccctacataccctaanttagggggatgaagatttactggtatttgatg

SEQ ID NO: 167 - DNA, 64 bp, Artificial Sequence
  Probe for Influenza A Virus H13N4
caaatatcccactacatacatatccccctaanttagggggatgaagatttactggtatttgatg

SEQ ID NO: 168 - DNA, 63 bp, Artificial Sequence
  Probe for Influenza A Virus H13N5
ccattccaattatctcggcaaaccccctaanttagggggatgaagatttactggtatttgatg

SEQ ID NO: 169 - DNA, 63 bp, Artificial Sequence
  Probe for Influenza A Virus H13N6
tgagtccttaatatatttcctgccccctaanttagggggatgaagatttactggtatttgatg

SEQ ID NO: 170 - DNA, 62 bp, Artificial Sequence
  Probe for Influenza A Virus H13N7
cctgcattaggtattttcaacatccctaanttagggggatgaagatttactggtatttgatg

SEQ ID NO: 171 - DNA, 59 bp, Artificial Sequence
  Probe for Influenza A Virus H13N8
ccacacatcacaatggagctccctaanttagggggatgaagatttactggtatttgatg

SEQ ID NO: 172 - DNA, 62 bp, Artificial Sequence
  Probe for Influenza A Virus H13N9
ttgtatagtgtgggtggtgatgaccctaanttagggggatgaagatttactggtatttgatg

SEQ ID NO: 173 - DNA, 61 bp, Artificial Sequence
  Probe for Influenza A Virus H14N1
cagaaattccaattgtcaaccaactccctaanttagggccatcaagcgataatgagcaaac

SEQ ID NO: 174 - DNA, 60 bp, Artificial Sequence
  Probe for Influenza A Virus H14N2
tacaagttccattgatacaaacgcccctaanttagggccatcaagcgataatgagcaaac

SEQ ID NO: 175 - DNA, 58 bp, Artificial Sequence
  Probe for Influenza A Virus H14N3
cccttccaattgtccctacataccctaanttagggccatcaagcgataatgagcaaac

SEQ ID NO: 176 - DNA, 61 bp, Artificial Sequence
  Probe for Influenza A Virus H14N4
caaatatcccactacatacatatccccctaanttagggccatcaagcgataatgagcaaac

SEQ ID NO: 177 - DNA, 60 bp, Artificial Sequence
  Probe for Influenza A Virus H14N5
ccattccaattatctcggcaaaccccctaanttagggccatcaagcgataatgagcaaac

SEQ ID NO: 178 - DNA, 60 bp, Artificial Sequence
  Probe for Influenza A Virus H14N6
tgagtccttaatatatttcctgccccctaanttagggccatcaagcgataatgagcaaac

SEQ ID NO: 179 - DNA, 59 bp, Artificial Sequence
  Probe for Influenza A Virus H14N7
cctgcattaggtattttcaacatccctaanttagggccatcaagcgataatgagcaaac

SEQ ID NO: 180 - DNA, 56 bp, Artificial Sequence
  Probe for Influenza A Virus H14N8
ccacacatcacaatggagctccctaanttagggccatcaagcgataatgagcaaac

SEQ ID NO: 181 - DNA, 59 bp, Artificial Sequence
  Probe for Influenza A Virus H14N9
ttgtatagtgtgggtggtgatgaccctaanttagggccatcaagcgataatgagcaaac

SEQ ID NO: 182 - DNA, 62 bp, Artificial Sequence
  Probe for Influenza A Virus H15N1
cagaaattccaattgtcaaccaactccctaanttaggggcatacaattgaccttgcagattc

SEQ ID NO: 183 - DNA, 61 bp, Artificial Sequence
  Probe for Influenza A Virus H15N2
tacaagttccattgatacaaacgcccctaanttaggggcatacaattgaccttgcagattc

SEQ ID NO: 184 - DNA, 59 bp, Artificial Sequence
  Probe for Influenza A Virus H15N3
cccttccaattgtccctacataccctaanttaggggcatacaattgaccttgcagattc

SEQ ID NO: 185 - DNA, 62 bp, Artificial Sequence
  Probe for Influenza A Virus H15N4
caaatatcccactacatacatatccccctaanttaggggcatacaattgaccttgcagattc

SEQ ID NO: 186 - DNA, 61 bp, Artificial Sequence
  Probe for Influenza A Virus H15N5
ccattccaattatctcggcaaaccccctaanttaggggcatacaattgaccttgcagattc

SEQ ID NO: 187 - DNA, 61 bp, Artificial Sequence
  Probe for Influenza A Virus H15N6
tgagtccttaatatatttcctgccccctaanttaggggcatacaattgaccttgcagattc

SEQ ID NO: 188 - DNA, 60 bp, Artificial Sequence
  Probe for Influenza A Virus H15N7
cctgcattaggtattttcaacatccctaanttaggggcatacaattgaccttgcagattc

SEQ ID NO: 189 - DNA, 57 bp, Artificial Sequence
  Probe for Influenza A Virus H15N8
ccacacatcacaatggagctccctaanttaggggcatacaattgaccttgcagattc

SEQ ID NO: 190 - DNA, 60 bp, Artificial Sequence
  Probe for Influenza A Virus H15N9
ttgtatagtgtgggtggtgatgaccctaanttaggggcatacaattgaccttgcagattc

SEQ ID NO: 191 - DNA, 62 bp, Artificial Sequence
  Probe for Influenza A Virus H16N1
cagaaattccaattgtcaaccaactccctaanttaggggacagaacattagacctgcatgat

SEQ ID NO: 192 - DNA, 61 bp, Artificial Sequence
  Probe for Influenza A Virus H16N2
tacaagttccattgatacaaacgcccctaanttaggggacagaacattagacctgcatgat

SEQ ID NO: 193 - DNA, 59 bp, Artificial Sequence
  Probe for Influenza A Virus H16N3
cccttccaattgtccctacataccctaanttaggggacagaacattagacctgcatgat

SEQ ID NO: 194 - DNA, 62 bp, Artificial Sequence
  Probe for Influenza A Virus H16N4
caaatatcccactacatacatatccccctaanttaggggacagaacattagacctgcatgat

SEQ ID NO: 195 - DNA, 61 bp, Artificial Sequence
  Probe for Influenza A Virus H16N5
ccattccaattatctcggcaaaccccctaanttaggggacagaacattagacctgcatgat

SEQ ID NO: 196 - DNA, 61 bp, Artificial Sequence
  Probe for Influenza A Virus H16N6
tgagtccttaatatatttcctgccccctaanttaggggacagaacattagacctgcatgat

SEQ ID NO: 197 - DNA, 60 bp, Artificial Sequence
  Probe for Influenza A Virus H16N7
cctgcattaggtattttcaacatccctaanttaggggacagaacattagacctgcatgat

SEQ ID NO: 198 - DNA, 57 bp, Artificial Sequence
  Probe for Influenza A Virus H16N8
ccacacatcacaatggagctccctaanttaggggacagaacattagacctgcatgat

SEQ ID NO: 199 - DNA, 60 bp, Artificial Sequence
  Probe for Influenza A Virus H16N9
ttgtatagtgtgggtggtgatgaccctaanttaggggacagaacattagacctgcatgat

SEQ ID NO: 200 - DNA, 22 bp, Artificial Sequence
  Forward Primer for Influenza A
gaccratcctgtcacctctgac

SEQ ID NO: 201 - DNA, 24 bp, Artificial Sequence
  Reverse Primer for Influenza A
agggcattytggacaaakcgtcta

SEQ ID NO: 202 - DNA, 23 bp, Artificial Sequence
  Forward Primer for Swine Influenza A
gcacggtcagcacttatyctrag

SEQ ID NO: 203 - DNA, 23 bp, Artificial Sequence
  Reverse Primer for Swine Influenza A
gtgrgctgggttttcatttggtc

SEQ ID NO: 204 - DNA, 23 bp, Artificial Sequence
  Forward Primer for Swine H1
gtgctataaacaccagcctycca

SEQ ID NO: 205 - DNA, 24 bp, Artificial Sequence
  Reverse Primer for Swine H1
cgggatattccttaatcctgtrgc

SEQ ID NO: 206 - DNA, 19 bp, Artificial Sequence
  Forward Primer for RNase P
agatttggacctgcgagcg

SEQ ID NO: 207 - DNA, 20 bp, Artificial Sequence
  Reverse Primer for RNase P
gagcggctgtctccacaagt

SEQ ID NO: 208 - DNA, 17 bp, Artificial Sequence
  Forward Primer for Influenza A H1H3
ggdaatytaatwgcdcc

SEQ ID NO: 209 - DNA, 20 bp, Artificial Sequence
  Reverse Primer for Influenza A H1H3
ggkayrtttctyagdcctgt

SEQ ID NO: 210 - DNA, 17 bp, Artificial Sequence
  Forward Primer for Influenza A N1N2
achcargagtcdgaatg

SEQ ID NO: 211 - DNA, 23 bp, Artificial Sequence
  Reverse Primer for Influenza A N1N2
gagccwtkccarttrtcyctgca

SEQ ID NO: 212 - DNA, 165 bp, Artificial Sequence
  Probe for beta-actin
gtcgatatcctgcaacgttccgctgacatcaccaccgccaaattgctccaacaccgtctcggcagcgctccctaaccctaanttagggttagggcctggcacccagcacaatgaagatcaagatcattgctcctcctgagcgcaagtactccgtgtggatcggcg

SEQ ID NO: 213 - DNA, 165 bp, Artificial Sequence
  Probe for EGFR
ggtcgatatcctgcaacgttccgctgacatcaccaccgccaaattgctccaacaccgtctcggcagcgctccctaaccctaanttagggttagggagccaggaacgtactggtgaaaacaccgcagcatgtcaagatcacagattttgggctggccaaactgctg

SEQ ID NO: 214 - DNA, 17 bp, Artificial Sequence
  Forward Primer for beta-actin
agcctcgcctttgccga

SEQ ID NO: 215 - DNA, 15 bp, Artificial Sequence
  Reverse Primer for beta-actin
ctggtgcctggggcg

SEQ ID NO: 216 - DNA, 22 bp, Artificial Sequence
  Probe for beta-actin
ccgccgcccgtccacacccgcc

SEQ ID NO: 217 - DNA, 21 bp, Artificial Sequence
  Forward Primer for EGFR
cgcagatagtcgcccaaagtt

SEQ ID NO: 218 - DNA, 21 bp, Artificial Sequence
  Reverse Primer for EGFR
gcattctttcatccccctgaa

SEQ ID NO: 219 - DNA, 21 bp, Artificial Sequence
  Probe for EGFR
cccgagacccccagcgctacc

SEQ ID NO: 220 - DNA, 75 bp, Artificial Sequence
  Probe for wild ACE
gccttgagctccagcccttagcccctaaccctaanttagggttagggccaaagtgctgggattacaggcgtgata

SEQ ID NO: 221 - DNA, 73 bp, Artificial Sequence
  Probe for ACE snp
gccttgagctccagcccttagcccctaaccctaanttagggttaggggacctgctgcctatacagtcactttt

SEQ ID NO: 222 - DNA, 63 bp, Artificial Sequence
  Probe for wild ADRB2
ggacgatgagagacatgacgaccctaaccctaanttagggttagggtcacgcagcaaagggac

SEQ ID NO: 223 - DNA, 63 bp, Artificial Sequence
  Probe for ADRB2 snp
ggacgatgagagacatgacgaccctaaccctaanttagggttagggtcacgcaggaaagggac

SEQ ID NO: 224 - DNA, 57 bp, Artificial Sequence
  Probe for wild Apo E
aggtgggaggcgaggccctaaccctaanttagggttagggaggacgtgtgcggccgc

SEQ ID NO: 225 - DNA, 57 bp, Artificial Sequence
  Probe for Apo E snp
aggtgggaggcgaggccctaaccctaanttagggttaggggaggacgtgcgcggccg

SEQ ID NO: 226 - DNA, 57 bp, Artificial Sequence
  Probe for wild Apo E
aggtgggaggcgaggccctaaccctaanttagggttagggtgcagaagcgcctggca

SEQ ID NO: 227 - DNA, 57 bp, Artificial Sequence
  Probe for Apo E snp
aggtgggaggcgaggccctaaccctaanttagggttagggtgcagaagtgcctggca

SEQ ID NO: 228 - DNA, 60 bp, Artificial Sequence
  Probe for wild CETP
ctatacctggctgtttgcccctaaccctaanttagggttagggtggggttcgagttaggg

SEQ ID NO: 229 - DNA, 60 bp, Artificial Sequence
  Probe for CETP snp
ctatacctggctgtttgcccctaaccctaanttagggttagggtggggttcaagttaggg

SEQ ID NO: 230 - DNA, 60 bp, Artificial Sequence
  Probe for wild CFH
acgtctatagatttacccccctaaccctaanttagggttagggcaaaatcatggaagaaa

SEQ ID NO: 231 - DNA, 60 bp, Artificial Sequence
  Probe for CFH snp
acgtctatagatttacccccctaaccctaanttagggttagggcaaaatcacggaagaaa

SEQ ID NO: 232 - DNA, 62 bp, Artificial Sequence
  Probe for wild ESR1
acatactacctgcaccagaaccctaaccctaanttagggttaggggtcccagctgttttatg

SEQ ID NO: 233 - DNA, 62 bp, Artificial Sequence
  Probe for ESR1 snp
acatactacctgcaccagaaccctaaccctaanttagggttaggggtcccagccgttttatg

SEQ ID NO: 234 - DNA, 60 bp, Artificial Sequence
  Probe for wild IL1A
ccctcaatcaaagtataaccctaaccctaanttagggttagggaaaaggtgctgacctag

SEQ ID NO: 235 - DNA, 60 bp, Artificial Sequence
  Probe for IL1A snp
ccctcaatcaaagtataaccctaaccctaanttagggttagggaaaaggtgatgacctag

SEQ ID NO: 236 - DNA, 60 bp, Artificial Sequence
  Probe for wild MTHFR
tcacctggatgggaaagaccctaaccctaanttagggttagggtgcgggagccgatttca

SEQ ID NO: 237 - DNA, 60 bp, Artificial Sequence
  Probe for MTHFR snp
tcacctggatgggaaagaccctaaccctaanttagggttagggtgcgggagtcgatttca

SEQ ID NO: 238 - DNA, 62 bp, Artificial Sequence
  Probe for wild NOS3
tttcctgccctctctttcctccctaaccctaanttagggttagggtgaggctggtgactaaa

SEQ ID NO: 239 - DNA, 62 bp, Artificial Sequence
  Probe for NOS3 snp
tttcctgccctctctttcctccctaaccctaanttagggttagggtgaggctgttgactaaa

SEQ ID NO: 240 - DNA, 25 bp, Artificial Sequence
  Forward Primer for ACE
ctggagagccactcccatcctttct

SEQ ID NO: 241 - DNA, 25 bp, Artificial Sequence
  Reverse Primer for ACE
gacgtggccatcacattcgtcagat

SEQ ID NO: 242 - DNA, 19 bp, Artificial Sequence
  Forward Primer for ADRB2
cttcttgctggcacccaat

SEQ ID NO: 243 - DNA, 20 bp, Artificial Sequence
  Reverse Primer for ADRB2
caggccagtgaagtgatgaa

SEQ ID NO: 244 - DNA, 21 bp, Artificial Sequence
  Forward Primer for Apo E
aatcggaactggaggaacaac

SEQ ID NO: 245 - DNA, 17 bp, Artificial Sequence
  Reverse Primer for Apo E
ggcctggtacactgcca

SEQ ID NO: 246 - DNA, 19 bp, Artificial Sequence
  Forward Primer for CETP
ctcgccttcaaggtcaagt

SEQ ID NO: 247 - DNA, 20 bp, Artificial Sequence
  Reverse Primer for CETP
tggctcagatctgaacccta

SEQ ID NO: 248 - DNA, 23 bp, Artificial Sequence
  Forward Primer for CFH
tcattgttatggtccttaggaaa

SEQ ID NO: 249 - DNA, 19 bp, Artificial Sequence
  Reverse Primer for CFH
actgtggtctgcgcttttg

SEQ ID NO: 250 - DNA, 20 bp, Artificial Sequence
  Forward Primer for ESR1
atccagggttatgtggcaat

SEQ ID NO: 251 - DNA, 20 bp, Artificial Sequence
  Reverse Primer for ESR1
tccttggcagattccatagc

SEQ ID NO: 252 - DNA, 30 bp, Artificial Sequence
  Forward Primer for IL1A
aatgaaaggaggggaggatgacagaaatgt

SEQ ID NO: 253 - DNA, 30 bp, Artificial Sequence
  Reverse Primer for IL1A
atggttttagaaatcatcaagcctaggtca

SEQ ID NO: 254 - DNA, 20 bp, Artificial Sequence
  Forward Primer for MTHFR
aggactctctctgcccagtc

SEQ ID NO: 255 - DNA, 20 bp, Artificial Sequence
  Reverse Primer for MTHFR
ggaagaactcagcgaactca

SEQ ID NO: 256 - DNA, 22 bp, Artificial Sequence
  Forward Primer for NOS3
cccctgagtcatctaagtattc

SEQ ID NO: 257 - DNA, 18 bp, Artificial Sequence
  Reverse Primer for NOS3
agctctggcacagtcaag

SEQ ID NO: 258 - DNA, 40 bp, Artificial Sequence
  Probe for wild K-ras
ccctaaccctaanttagggttagggggagctggtggcgta

SEQ ID NO: 259 - DNA, 40 bp, Artificial Sequence
  Probe for mutant K-ras
ccctaaccctaanttagggttagggggagctagtggcgta

SEQ ID NO: 260 - DNA, 40 bp, Artificial Sequence
  Probe for mutant K-ras
ccctaaccctaanttagggttagggggagctcgtggcgta

SEQ ID NO: 261 - DNA, 40 bp, Artificial Sequence
  Probe for mutant K-ras
ccctaaccctaanttagggttagggggagcttgtggcgta

SEQ ID NO: 262 - DNA, 40 bp, Artificial Sequence
  Probe for mutant K-ras
ccctaaccctaanttagggttagggggagctgatggcgta

SEQ ID NO: 263 - DNA, 41 bp, Artificial Sequence
  Probe for mutant K-ras
ccctaaccctaanttagggttagggtggagctgctggcgta

SEQ ID NO: 264 - DNA, 40 bp, Artificial Sequence
  Probe for mutant K-ras
ccctaaccctaanttagggttagggggagctgttggcgta

SEQ ID NO: 265 - DNA, 43 bp, Artificial Sequence
  Probe for Positive Control
ccctaaccctaanttagggttaggggccttgacgatacagcta

SEQ ID NO: 266 - DNA, 58 bp, Artificial Sequence
  Probe for wild K-ras
tagctgtatcgtcaaggcccctaaccctaanttagggttagggggagctggtggcgta

SEQ ID NO: 267 - DNA, 58 bp, Artificial Sequence
  Probe for mutant K-ras
tagctgtatcgtcaaggcccctaaccctaanttagggttagggggagctagtggcgta

SEQ ID NO: 268 - DNA, 58 bp, Artificial Sequence
  Probe for mutant K-ras
tagctgtatcgtcaaggcccctaaccctaanttagggttagggggagctcgtggcgta

SEQ ID NO: 269 - DNA, 58 bp, Artificial Sequence
  Probe for mutant K-ras
tagctgtatcgtcaaggcccctaaccctaanttagggttagggggagcttgtggcgta

SEQ ID NO: 270 - DNA, 58 bp, Artificial Sequence
  Probe for mutant K-ras
tagctgtatcgtcaaggcccctaaccctaanttagggttagggggagctgatggcgta

SEQ ID NO: 271 - DNA, 59 bp, Artificial Sequence
  Probe for mutant K-ras
tagctgtatcgtcaaggcccctaaccctaanttagggttagggtggagctgctggcgta

SEQ ID NO: 272 - DNA, 58 bp, Artificial Sequence
  Probe for mutant K-ras
tagctgtatcgtcaaggcccctaaccctaanttagggttagggggagctgttggcgta